(19) United States
(12) Patent Application Publication
(10) Pub. No.: US 2019/0077809 A1
(43) Pub. Date: Mar. 14, 2019

(54) Spirocyclic Derivatives

(71) Applicant: BASF SE, Ludwigshafen (DE)

(72) Inventors: Pascal BINDSCHAEDLER, Limburgerhof (DE); Karsten KOERBER, Ludwigshafen (DE); Wolfgang VON DEYN, Ludwigshafen (DE); Franz-Josef BRAUN, Research Triangle Park, NC (US)

(21) Appl. No.: 16/082,715

(22) PCT Filed: Mar. 1, 2017
(86) PCT No.: PCT/EP2017/054731

§ 371 Date: Sep. 6, 2018

(30) Foreign Application Priority Data
Mar. 9, 2016 (EP) 16159401.5

(51) Int. Cl. C07D 491/107; C07D 495/10; A01N 43/90

(52) U.S. Cl. CPC C07D 491/107; A01N 43/90; C07D 495/10

(57) ABSTRACT

The present invention relates to novel spiro compounds of formula I,

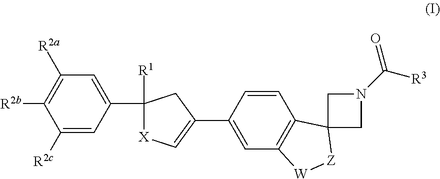

wherein the variables have the meanings as defined in the specification, to compositions comprising them, to active compound combinations comprising them, and to their use for protecting growing plants from attack or infestation by invertebrate pests, furthermore, to seed comprising such compounds.

### DESCRIPTION

The present invention relates to spirocyclic compounds of formula I

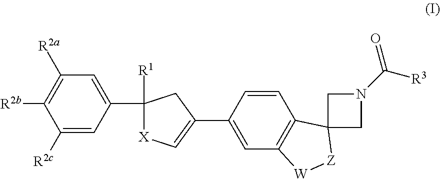

wherein
X CH₂ or S;
W-Z are #-CH₂—S(O)ₙ—*, or #-C(═O)—O—*, wherein # marks the bond of W to phenyl, and * the bond of Z to azetidin;
   n is 0, 1, or 2;
R¹ halomethyl;
R^{2a}, halogen, halomethyl, halomethoxy;
R^{2b}, R^{2c} are independently H, or as defined for R^{2a};
R³ is selected from H, C₁-C₆-alkyl, C₁-C₆-haloalkyl, C₂-C₆-alkenyl, C₂-C₆-haloalkenyl, C₂-C₆-alkynyl, C₂-C₆-haloalkynyl which aliphatic groups are unsubstituted or substituted by one or more radicals R³¹; C₃-C₆-cycloalkyl, C₃-C₆-halocycloalkyl which cyclic groups are unsubstituted or substituted by one or more radicals R³²; C(═O)N(R³³)R³⁴, N(R³³)R³⁵, CH—NOR³⁶; phenyl, heterocyclyl, or hetaryl which rings are unsubstituted or partially or fully substituted by R^{A};
   R³¹ is independently OH, cyano, C₁-C₆-alkoxy, C₁-C₆-haloalkoxy, S(O)ₙ—C₁-C₆alkyl, S(O)ₙ—C₁-C₆-haloalkyl, C(═O)N(R³³)R³⁴, C₃-C₆-cycloalkyl, or C₃-C₆-halocycloalkyl which cycles are unsubstituted or substituted by one or more R³¹¹; or phenyl, heterocyclyl or hetaryl which rings are unsubstituted or partially or fully substituted by R^{A};
      R³¹¹ is independently OH, cyano, C₁-C₂-alkyl, or C₁-C₂-haloalkyl;
   R³³ is H, or C₁-C₆-alkyl,
   R³⁴ is H, C₁-C₆-alkyl, C₁-C₆-haloalkyl, C₂-C₆-alkenyl, C₂-C₆-haloalkenyl, C₂-C₆-alkynyl, C₂-C₆-haloalkynyl, or C₃-C₆-cycloalkyl, C₃-C₆-halocycloalkyl, C₃-C₆-cycloalkylmethyl, or C₃-C₆-halocycloalkylmethyl which rings are unsubstituted or substituted by a cyano;
   R³⁵ H, C₁-C₆-alkyl, C₁-C₆-haloalkyl, C₂-C₄-alkenyl, C₂-C₄-alkynyl, CH₂—CN, C₃-C₆-cycloalkyl, C₃-C₆-halocycloalkyl, C₃-C₆-cycloalkylmethyl, C₃-C₆-halocycloalkylmethyl, phenyl and hetaryl which aromatic rings are unsubstituted or partially or fully substituted by R^{A};
   R³² C₁-C₆-alkyl, C₁-C₆-haloalkyl, or a group as defined for R³¹;
   R³⁶ is independently H, C₁-C₆-alkyl, or C₁-C₆-haloalkyl;
   R^{A} is independently selected from halogen, cyano, NO₂, C₁-C₄-alkyl, C₁-C₄-haloalkyl, C₂-C₄-alkenyl, C₂-C₄-haloalkenyl, C₂-C₄-alkynyl, C₂-C₄-haloalkynyl, C₃-C₆-cycloalkyl, C₃-C₆-halocycloalkyl, C₁-C₄-alkoxy, C₁-C₄-haloalkoxy, S(O)ₙ—C₁-C₄-alkyl, S(O)ₙ—C₁-C₄-haloalkyl, C₁-C₄-alkylcarbonyl, C₁-C₄-haloalkylcarbonyl, C(═O)N(R³³)R³⁴; or
   two R^{A} present on the same carbon atom of a saturated or partially saturated ring may form together ═O or ═S; or
   two R^{A} present on the same S or SO ring member of a heterocyclic ring may together form a group ═N(C₁-C₆-alkyl), ═NO(C₁-C₆-alkyl), ═NN(H)(C₁-C₆-alkyl) or ═NN(C₁-C₆-alkyl)₂;
and the N-oxides, stereoisomers and agriculturally or veterinarily acceptable salts thereof.

Moreover, the invention relates to processes and intermediates for preparing the compounds of formula I, and also to active compound combinations, and compositions comprising them, and to their use for protecting growing plants from attack or infestation by invertebrate pests. Furthermore, the invention relates to methods of applying such compounds. The present invention also relates to seed comprising such compounds.

Invertebrate pests and in particular arthropods and nematodes destroy growing and harvested crops and attack wooden dwelling and commercial structures, thereby causing large economic loss to the food supply and to property. There is an ongoing need for new agents for combating invertebrate pests such as insects, arachnids and nematodes.

WO 2015/169723, WO 2015/104422, WO 2014/072480 and WO 2015/114157 describe derivatives of cyclopentenes and dihydrothiophenes. These compounds are mentioned to be useful for combating invertebrate pests.

Nevertheless, there remains a need for highly effective and versatile agents for combating invertebrate pests. It is therefore an object of the present invention to provide compounds having a good pesticidal activity and showing a broad activity spectrum against a large number of different invertebrate pests, especially against difficult to control pests, such as insects.

It has been found that these objects can be achieved by compounds of formula I as depicted and defined below, and by their stereoisomers, salts, tautomers and N-oxides, in particular their agriculturally acceptable salts.

Compounds of formula I wherein X is S, and W-Z is #-CH₂—O—*, or ′#-CH₂—S(O)ₙ—* can be prepared by the following route. Such compounds correspond to formula I.A. They can be prepared starting from an enone of formula II. The azetidine-N is protected by any suitable protecting group (e.g. Boc in the sketch below). Alternatively, the azetidine-N may be substituted with C(O)R³. The synthesis of enones of formula II, wherein Z is O, is known from WO 2014/081800, page 13. The synthesis of formula II compounds wherein Z is S is known from WO 2014/039484, page 29.

The route to compounds of formula IA, wherein X is S, starts with the reaction of enones of formula II with alkylthiogycolates of formula IIa, wherein R^{E} is C₁-C₈-alkyl, to obtain esters of formula III.

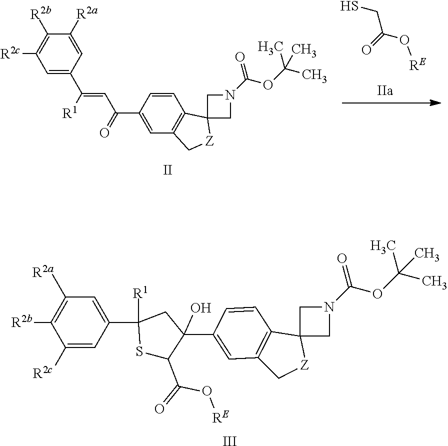

This transformation is usually carried out at temperatures of from −20° C. to 100° C., preferably from 0° C. to 40° C., in an inert solvent such as tetrahydrofurane (THF), dioxane or toluene, in the presence of organic bases, for example tertiary amines, such as trimethylamine, triethylamine, triisopropylethylamine and N-methylpiperidine, pyridine, substituted pyridines, such as collidine, lutidine and 4-dimethylaminopyridine, and also bicyclic amines, or inorganic bases such as alkali metal and alkaline earth metal oxides, such as lithium oxide, sodium oxide, calcium oxide, and magnesium oxide (c.f. WO 2015/169723).

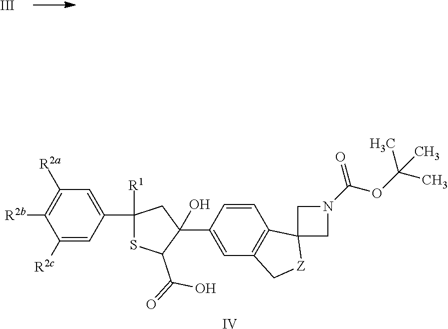

Esters of formula III are hydrolyzed to obtain compounds of formula IV.

This transformation is usually carried out at temperature from −20° C. to 120° C., preferably at 0° C. to 100° C., in a water and a base such as an alkali metal hydroxide such as potassium hydroxide, sodium hydroxide or lithium hydroxide, in the absence or in the presence of a solvent, such as THF, dioxane, or methanol (c.f. WO 2015/169723).

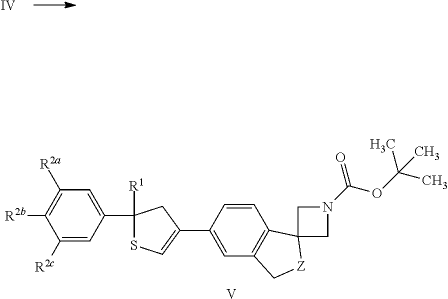

Compounds of formula V can be prepared by dehydrative decarboxylation of compounds of formula IV with reagents such as dimethylformamide dineopentylacetal in a solvent such as dimethylsulfoxide (DMSO), dimethylformamide (DMF), or N-methylpyrrolidone (NMP). The reaction is carried out at temperature from 25° C. to 250° C., preferably at 100° C. to 200° C. (c.f. WO 2014/072480).

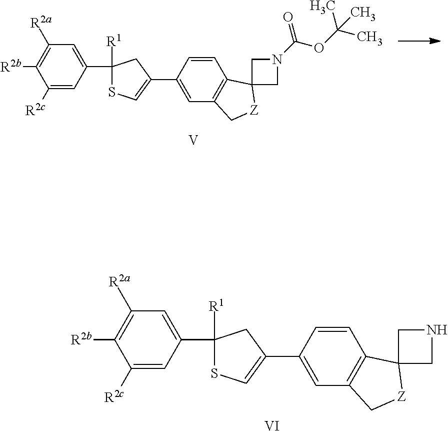

Compounds of formula VI can be obtained from compounds V by hydrolysis of the protective group. This transformation is usually carried out at temperatures of from −20° C. to 100° C., preferably from 0° C. to 40° C., using trifluoroacetic acid as a co-solvent in a solvent such as dichloromethane or hydrochloric acid in ethers such as diethylether, Diisopropylether, tert.-butylmethylether, dioxane, and tetrahydrofurane (c.f. WO 2015/104422).

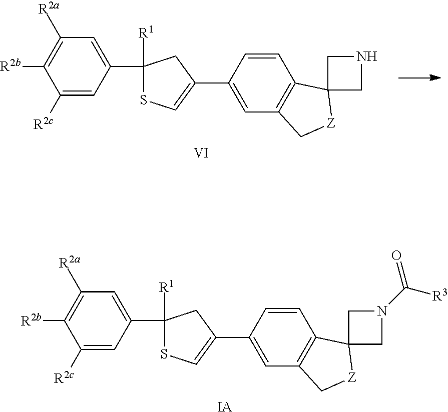

Compounds of formula IA are obtained from compounds of formula VI in an amidation reaction. This transformation is usually carried out at temperatures of from −20° C. to 100° C., preferably from 0° C. to 40° C. (c.f. WO 2015/104422).

Thereby compounds of formula IA are reacted with acid chloride R³-COCl in a solvent such as halogenated hydrocarbons such as methylene chloride, or ethers such as diethylether, diisopropylether, tert.-butylmethylether, dioxane, anisole, and THF, in the presence of an organic bases, for example tertiary amines, such as trimethylamine, triethylamine, triisopropylethylamine and N-methylpiperidine, pyridine, substituted pyridines, such as collidine, lutidine and 4-dimethylaminopyridine, and also bicyclic amines.

Compounds of formula I wherein X is CH₂, and W-Z is #-CH₂—O—* or ′#-CH₂—S(O)ₙ—* can be prepared by the following route. Such compounds correspond to formula I.B. Such compounds can be prepared starting from an enone of formula II that are reacted with nitroethane to obtain compounds of formula VII.

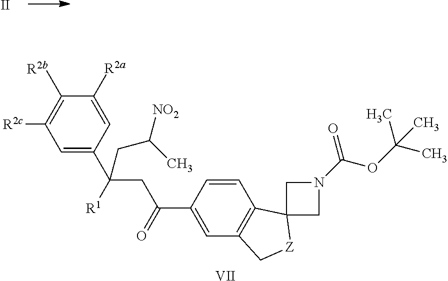

This transformation is usually carried out at temperatures of from −40° C. to 80° C., preferably from 0° C. to 40° C. in a solvent such as acetonitrile, THF, dioxane or toluene, in the presence of organic bases, for example amines, such as trimethylamine, triethylamine, triisopropylethylamine and N-methylpiperidine, pyridine, substituted pyridines, such as collidine, lutidine and 4-dimethylaminopyridine, and also bicyclic amines such as 1,8-diazabicyclo[5.4.0]undec-7-ene (DBU) (c.f. Angew. Chem. 2013, Vol. 125, p. 5685, DOI: 10.1002/ange.201301123).

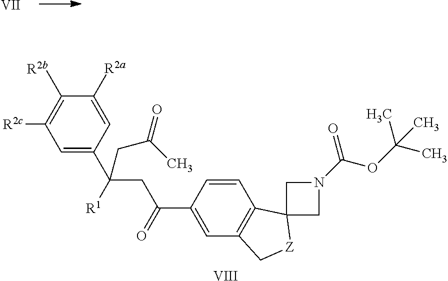

Compounds of formula VII are then reacted with catalytic or stoichiometric tetrapropyl-ammonium perruthenate (TPAP) to obtain compounds of formula VIII. This transformation is usually carried out at temperatures of from −20° C. to 80° C., preferably from 0° C. to 40° C. in an inert solvent such as dichloromethane or acetonitrile. If catalytic amounts of TPAP are used, hydroperoxides or N-methylmorpholine N-oxide (NMO) are used as co-oxidants (c.f. Chem. Rev., 1955, Vol. 55, p. 137. DOI: 10.1021/cr50001a003).

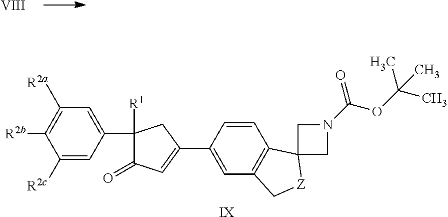

Compounds of formula VIII are then converted to compounds of formula IX. This transformation is usually carried out at temperatures of from −40° C. to 150° C., preferably from 20° C. to 100° C. in an aliphatic hydrocarbons such as pentane, hexane, cyclohexane, heptane and petrol ether, or aromatic hydrocarbons such as toluene, o-, m-, and p-xylene, in the presence of organic bases, for example amines, such as trimethylamine, triethylamine, triisopropylethylamine and N-methylpiperidine, pyridine, substituted pyridines, such as collidine, lutidine and 4-dimethylaminopyridine, and also bicyclic amines. Alternatively, alkali metal and alkaline earth metal carbonates, such as lithium carbonate, potassium carbonate and calcium carbonate or alkali metal alkylates such as sodium methylate are used (c.f. WO 2009/012954).

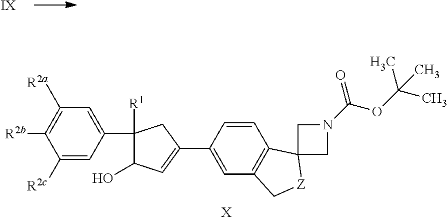

Compounds of formula IX are then converted to compounds of formula X using a Luche reduction. This transformation is carried out at temperatures form −40° C. to 100° C., preferably from −20° C. to 40° C. As a solvent, mixtures of ethers such as diethylether, diisopropylether, tert.-butylmethylether, dioxane, anisole, and tetrahydrofurane and alcohols such as methanol, ethanol, n-propanol, isopropanol, n-butanol, and tert.-butanol, in the presence of an alkali metal hydride such as sodium borohydride and a Lewis acid such as cerium trichloride (CeCl₃) may be used (c.f. JACS, 1981, Vol 103, p. 5454, DOI: 10.1021/ja00408a029).

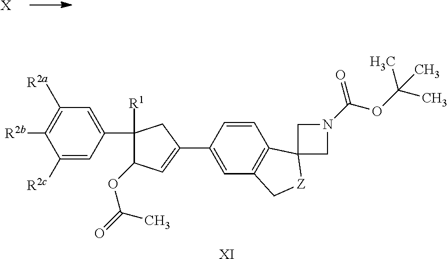

Compounds of formula X are then acetylated to yield compounds of formula Xl. This transformation is carried out at temperatures from −40° C. to 100° C., preferably form −20° C. to 40° C. in an inert solvent such as dichloromethane, toluene or THF, using acetyl chloride in the presence of an organic bases, for example amines, such as trimethylamine, triethylamine, triisopropylethylamine and N-methylpiperidine, pyridine, substituted pyridines, such as collidine, lutidine and 4-dimethylaminopyridine, and also bicyclic amines or acetic anhydride. Optionally, a catalytic amount of DMAP may be added (c.f. Canadian Journal of Chemistry, 1989, Vol. 67, p. 580. DOI: 10.1139/v89-088).

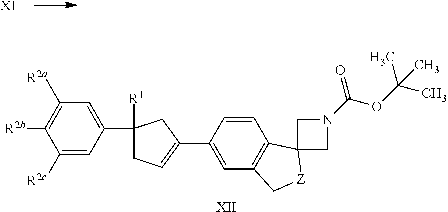

Compounds of formula XI are then converted to compounds of formula XII. This transformation is usually carried out at temperatures of from −40° C. to 100° C., preferably from 0° C. to 40° C. in a solvent such as ethers such as diethylether, diisopropylether, tert.-butylmethylether, dioxane, anisole, and THF in the presense of a palladium catalyst such as tetrakis(triphenylphosphine)palladium (Pd(Ph₃P)₄) and an alkali metal hydride such as sodium borohydride (c.f. Canadian Journal of Chemistry, 1989, Vol. 67, p. 580. DOI: 10.1139/v89-088).

The elimination of the protecting group fo formula XII and acylation with acid chloride R³-COCl to yield compounds of formula IB proceed analogously to the above described reactions of formulae V and VI, resp.

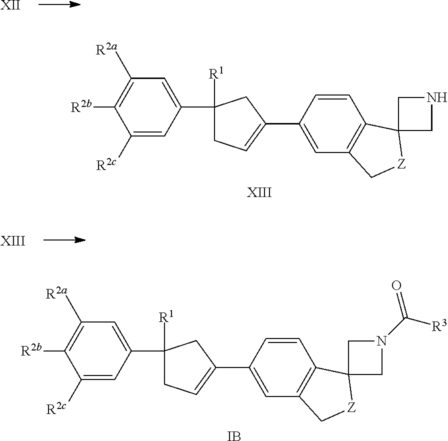

Compounds of formula I wherein W-Z is #-C(═O)—O—* can be prepared by the following route. Such compounds correspond to formula I.C.

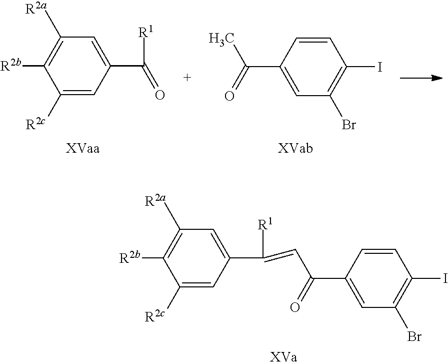

Enones of formula XVa can be prepared by an aldol condensation of commercially available trifluoroketones of formula XVaa (for R¹═CF₃) and acetophenone XVab (c.f. Chemistry—a European Journal, 2011, Vol. 17, p. 2099). This aldol condensation is performed according to WO 2012/120399, page 93.

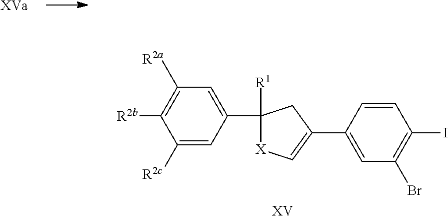

Enones of formula XVa are transformed to compounds of formula XV in analogy to sequence from compound II to compound V, and sequence from compound II to compound XII, resp.

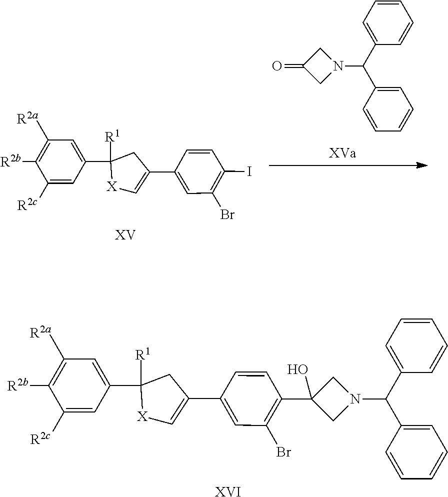

Compounds of formula XV are reacted with compounds of formula XVa to obtain compounds of formula XVI. This transformation is usually carried out at temperatures from −100° C. to 20° C., preferably form −78° C. to 0° C. in a suitable solvent such as ethers such as diethylether, diisopropylether, tert.-butylmethylether, dioxane, anisole, and THF, in the presence of a Grignard reagent such as isopropylmagnesium chloride or a alkyl lithium reagent such as n-BuLi, sec-BuLi or tert.-BuLi. The azetidine-N is protected by any suitable protecting group (e.g. CH(Ph)₂, as shown for the sketch above). Alternatively, the azetidine-N may be substituted with C(O)R³. (c.f. WO 2012/120399)

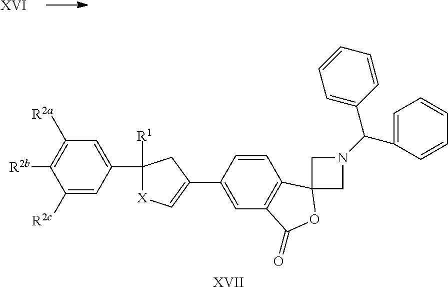

Compounds of formula XVI are reacted with a reagent such as Zn(CN)₂ to yield compounds of formula XVII. This transformation is carried out at temperatures from 0° C. to 200° C., preferably form 70° C. to 150° C. in a solvent such as DMSO, DMF, and dimethylacetamide (DMA) in the presence of a palladium catalyst such as Pd(Ph₃P)₄ (c.f. WO 2012/120399).

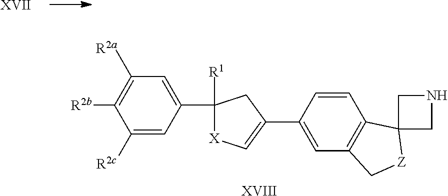

Compounds of formula XVII are then reacted with chloroethyl chloroformate to give compounds of formula XVIII. This transformation is usually carried out at temperature from 0° C. to 150° C., preferably from 30° C. to 80° C. in a suitable solvent such as acetonitrile, dichloromethane or mixtures thereof. (c.f. WO 2012/120399).

Acylation with acid chloride R³—COCl to yield compounds of formula I.C proceed analogously to the above described reactions of formulae V and VI, resp.

The starting materials required for preparing the compounds I are commercially available or known from the literature, or can be prepared in accordance with the literature cited.

The reaction mixtures are worked up in a customary manner, for example by mixing with water, separating the phases and, if appropriate, chromatographic purification of the crude products. Some of the intermediates and end products are obtained in the form of colorless or slightly brownish viscous oils which are purified or freed from volatile components under reduced pressure and at moderately elevated temperature. If the intermediates and end products are obtained as solids, purification can also be carried out by recrystallization or digestion.

If individual compounds I cannot be obtained by the routes described above, they can be prepared by derivatization of other compounds I.

However, if the synthesis yields mixtures of isomers, a separation is generally not necessarily required since in some cases the individual isomers can be interconverted during work-up for use or during application (for example under the action of light, acids or bases). Such conversions may also take place after use, for example in the treatment of plants in the treated plant, or in the harmful fungus to be controlled.

The radicals attached to the backbone of formula I may contain one or more centers of chirality. In this case the compounds of formula I are present in the form of different enantiomers or diastereomers, depending on the substituents. The present invention relates to every possible stereoisomer of the formula I, i.e. to single enantiomers or diastereomers, as well as to mixtures thereof.

As already indicated above, the compounds of formula I may be present in the form of different structural isomers depending on the position of R¹. The present invention relates to every possible structural isomer as indicated in the compounds of formula I, and mixtures thereof.

The compounds of formula I may be amorphous or may exist in one or more different crystalline states (polymorphs) which may have different macroscopic properties such as stability or show different biological properties such as activities. The present invention relates to amorphous and crystalline compounds of formula I, mixtures of different crystalline states of the respective compound I, as well as amorphous or crystalline salts thereof.

Salts of the compounds of the formula I are preferably veterinary and/or agriculturally acceptable salts, preferably agriculturally acceptable salts. They can be formed in a customary manner, e.g. by reacting the compound with an acid of the anion in question if the compound of formula I has a basic functionality.

Veterinary and/or agriculturally useful salts of the compounds of formula I encompass especially the acid addition salts of those acids whose cations and anions, respectively, have no adverse effect on the pesticidal action of the compounds of formula I.

Anions of useful acid addition salts are primarily chloride, bromide, fluoride, hydrogensulfate, sulfate, dihydrogenphosphate, hydrogenphosphate, phosphate, nitrate, bicarbonate, carbonate, hexafluorosilicate, hexafluorophosphate, benzoate, and the anions of C₁C₄-alkanoic acids, preferably formate, acetate, propionate and butyrate. They can be formed by reacting compounds of formula I with an acid of the corresponding anion, preferably of hydrochloric acid, hydrobromic acid, sulfuric acid, phosphoric acid or nitric acid.

The term “N-oxide” includes any compound of formula I which has at least one tertiary nitrogen atom that is oxidized to an N-oxide moiety.

The organic moieties mentioned in the above definitions of the variables are—like the term halogen—collective terms for individual listings of the individual group members. The prefix Cₙ-Cₘ indicates in each case the possible number of carbon atoms in the group.

The term “halogen” denotes in each case fluorine, bromine, chlorine or iodine, in particular fluorine, chlorine or bromine.

The term “alkyl” as used herein and in the alkyl moieties of alkylamino, alkylcarbonyl, alkylthio, alkylsulfinyl, alkylsulfonyl and alkoxyalkyl denotes in each case a straight-chain or branched alkyl group having usually from 1 to 10 carbon atoms, frequently from 1 to 6 carbon atoms, preferably 1 to 4 carbon atoms, more preferably from 1 to 3 carbon atoms. Examples of an alkyl group are methyl, ethyl, n-propyl, iso-propyl, n-butyl, 2-butyl, iso-butyl, tert-butyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, and 1-ethyl-2-methylpropyl.

The term “haloalkyl” as used herein and in the haloalkyl moieties of haloalkylcarbonyl, haloalkoxycarbonyl, haloalkylthio, haloalkylsulfonyl, haloalkylsulfinyl, haloalkoxy and haloalkoxyalkyl, denotes in each case a straight-chain or branched alkyl group having usually from 1 to 10 carbon atoms, frequently from 1 to 6 carbon atoms, preferably from 1 to 4 carbon atoms, wherein the hydrogen atoms of this group are partially or totally replaced with halogen atoms. Preferred haloalkyl moieties are selected from C₁-C₄-haloalkyl, more preferably from C₁-C₃-haloalkyl or C₁-C₂-haloalkyl, in particular from C₁-C₂-fluoroalkyl such as fluoromethyl, difluoromethyl, trifluoromethyl, 1-fluoroethyl, 2-fluoroethyl, 2,2-difluoroethyl, 2,2,2-trifluoroethyl, pentafluoroethyl, and the like.

The term “alkoxy” as used herein denotes in each case a straight-chain or branched alkyl group which is bonded via an oxygen atom and has usually from 1 to 10 carbon atoms, frequently from 1 to 6 carbon atoms, preferably 1 to 4 carbon atoms. Examples of an alkoxy group are methoxy, ethoxy, n-propoxy, iso-propoxy, n-butyloxy, 2-butyloxy, iso-butyloxy, tert.-butyloxy, and the like.

The term “alkoxyalkyl” as used herein refers to alkyl usually comprising 1 to 10, frequently 1 to 4, preferably 1 to 2 carbon atoms, wherein 1 carbon atom carries an alkoxy radical usually comprising 1 to 4, preferably 1 or 2 carbon atoms as defined above. Examples are CH₂OCH₃, CH₂—OC₂H₅, 2-(methoxy)ethyl, and 2-(ethoxy)ethyl.

The term “haloalkoxy” as used herein denotes in each case a straight-chain or branched alkoxy group having from 1 to 10 carbon atoms, frequently from 1 to 6 carbon atoms, preferably 1 to 4 carbon atoms, wherein the hydrogen atoms of this group are partially or totally replaced with halogen atoms, in particular fluorine atoms. Preferred haloalkoxy moieties include C₁-C₄-haloalkoxy, in particular C₁-C₂-fluoroalkoxy, such as fluoromethoxy, difluoromethoxy, trifluoromethoxy, 1-fluoroethoxy, 2-fluoroethoxy, 2,2-difluoroethoxy, 2,2,2-trifluoroethoxy, 2-chloro-2-fluoroethoxy, 2-chloro-2,2-difluoro-ethoxy, 2,2dichloro-2-fluorethoxy, 2,2,2-trichloroethoxy, pentafluoroethoxy and the like.

The term “alkylthio “(alkylsulfanyl: S-alkyl)” as used herein refers to a straight-chain or branched saturated alkyl group having 1 to 10 carbon atoms, preferably 1 to 4 carbon atoms (═C₁C₄-alkylthio), more preferably 1 to 3 carbon atoms, which is attached via a sulfur atom.

The term “haloalkylthio” as used herein refers to an alkylthio group as mentioned above wherein the hydrogen atoms are partially or fully substituted by fluorine, chlorine, bromine and/or iodine.

The term “alkylsulfinyl” (alkylsulfoxyl: S(═O)-C₁-C₆-alkyl), as used herein refers to a straight-chain or branched saturated alkyl group (as mentioned above) having 1 to 10 carbon atoms, preferably 1 to 4 carbon atoms (═C₁-C₄-alkylsulfinyl), more preferably 1 to 3 carbon atoms bonded through the sulfur atom of the sulfinyl group at any position in the alkyl group.

The term “haloalkylsulfinyl” as used herein refers to an alkylsulfinyl group as mentioned above wherein the hydrogen atoms are partially or fully substituted by fluorine, chlorine, bromine and/or iodine.

The term “alkylsulfonyl” (S(═O)₂-alkyl) as used herein refers to a straight-chain or branched saturated alkyl group having 1 to 10 carbon atoms, preferably 1 to 4 carbon atoms (═C₁-C₄-alkylsulfonyl), preferably 1 to 3 carbon atoms, which is bonded via the sulfur atom of the sulfonyl group at any position in the alkyl group.

The term “haloalkylsulfonyl” as used herein refers to an alkylsulfonyl group as mentioned above wherein the hydrogen atoms are partially or fully substituted by fluorine, chlorine, bromine and/or iodine.

The term “alkylcarbonyl” refers to an alkyl group as defined above, which is bonded via the carbon atom of a carbonyl group (C═O) to the remainder of the molecule.

The term “haloalkylcarbonyl” refers to an alkylcarbonyl group as mentioned above, wherein the hydrogen atoms are partially or fully substituted by fluorine, chlorine, bromine and/or iodine.

The term “alkoxycarbonyl” refers to an alkylcarbonyl group as defined above, which is bonded via an oxygen atom to the remainder of the molecule.

The term “haloalkoxycarbonyl” refers to an alkoxycarbonyl group as mentioned above, wherein the hydrogen atoms are partially or fully substituted by fluorine, chlorine, bromine and/or iodine.

The term “alkenyl” as used herein denotes in each case a singly unsaturated hydrocarbon radical having usually 2 to 10, frequently 2 to 6, preferably 2 to 4 carbon atoms, e.g. vinyl, allyl (2-propen-1-yl), 1-propen-1-yl, 2-propen-2-yl, methallyl (2-methylprop-2-en-1-yl), 2-buten-1-yl, 3-buten-1-yl, 2-penten-1-yl, 3-penten-1-yl, 4-penten-1-yl, 1-methylbut-2-en-1-yl, 2-ethylprop-2-en-1-yl and the like.

The term “haloalkenyl” as used herein refers to an alkenyl group as defined above, wherein the hydrogen atoms are partially or totally replaced with halogen atoms.

The term “alkynyl” as used herein denotes in each case a singly unsaturated hydrocarbon radical having usually 2 to 10, frequently 2 to 6, preferably 2 to 4 carbon atoms, e.g. ethynyl, propargyl (2-propyn-1-yl), 1-propyn-1-yl, 1-methylprop-2-yn-1-yl), 2-butyn-1-yl, 3-butyn-1-yl, 1-pentyn-1-yl, 3-pentyn-1-yl, 4-pentyn-1-yl, 1-methylbut-2-yn-1-yl, 1-ethylprop-2-yn-1-yl and the like.

The term “haloalkynyl” as used herein refers to an alkynyl group as defined above, wherein the hydrogen atoms are partially or totally replaced with halogen atoms.

The term “cycloalkyl” as used herein and in the cycloalkyl moieties of cycloalkoxy and cycloalkylthio denotes in each case a monocyclic cycloaliphatic radical having usually from 3 to 10 or from 3 to 6 carbon atoms, such as cyclopropyl, cyclobutyl, cyclopentyl, cyclohexyl, cycloheptyl, cyclooctyl, cyclononyl and cyclodecyl or cyclopropyl, cyclobutyl, cyclopentyl and cyclohexyl.

The term “halocycloalkyl” as used herein and in the halocycloalkyl moieties of halocycloalkoxy and halocycloalkylthio denotes in each case a monocyclic cycloaliphatic radical having usually from 3 to 10 C atoms or 3 to 6 C atoms, wherein at least one, e.g. 1, 2, 3, 4 or 5 of the hydrogen atoms, are replaced by halogen, in particular by fluorine or chlorine. Examples are 1- and 2-fluorocyclopropyl, 1,2-, 2,2- and 2,3-difluorocyclopropyl, 1,2,2-trifluorocyclopropyl, 2,2,3,3-tetrafluorocyclpropyl, 1- and 2-chlorocyclopropyl, 1,2-, 2,2- and 2,3-dichlorocyclopropyl, 1,2,2-trichlorocyclopropyl, 2,2,3,3-tetrachlorocyclpropyl, 1-,2- and 3-fluorocyclopentyl, 1,2-, 2,2-, 2,3-, 3,3-, 3,4-, 2,5-difluorocyclopentyl, 1-,2- and 3-chlorocyclopentyl, 1,2-, 2,2-, 2,3-, 3,3-, 3,4-, 2,5-dichlorocyclopentyl and the like.

The term “cycloalkenyl” as used herein and in the cycloalkenyl moieties of cycloalkenyloxy and cycloalkenylthio denotes in each case a monocyclic singly unsaturated non-aromatic radical having usually from 3 to 10, e.g. 3 or 4 or from 5 to 10 carbon atoms, preferably from 3- to 8 carbon atoms. Exemplary cycloalkenyl groups include cyclopropenyl, cycloheptenyl or cyclooctenyl.

The term “halocycloalkenyl” as used herein and in the halocycloalkenyl moieties of halocycloalkenyloxy and halocycloalkenylthio denotes in each case a monocyclic singly unsaturated nonaromatic radical having usually from 3 to 10, e.g. 3 or 4 or from 5 to 10 carbon atoms, preferably from 3- to 8 carbon atoms, wherein at least one, e.g. 1, 2, 3, 4 or 5 of the hydrogen atoms, are replaced by halogen, in particular by fluorine or chlorine. Examples are 3,3,-difluorocyclopropen-1-yl and 3,3-dichlorocyclopropen-1-yl.

The term “cycloalkenylalkyl” refers to a cycloalkenyl group as defined above which is bonded via an alkylene group, such as a C₁-C₅-alkyl group or a C₁-C₄-alkyl group, in particular a methylene group (=cycloalkenylmethyl), to the remainder of the molecule.

The term “carbocycle” or “carbocyclyl” includes in general a 3- to 12-membered, preferably a 3- to 8-membered or a 5- to 8-membered, more preferably a 5- or 6-membered mono-cyclic, non-aromatic ring comprising 3 to 12, preferably 3 to 8 or 5 to 8, more preferably 5 or 6 carbon atoms. Preferably, the term “carbocycle” covers cycloalkyl and cycloalkenyl groups as defined above.

The term “heterocycle” or “heterocyclyl” includes in general 3- to 12-membered, preferably 5-or 6-membered, in particular 6-membered monocyclic heterocyclic non-aromatic radicals. The heterocyclic non-aromatic radicals usually comprise 1, 2 or 3 heteroatoms selected from N, O and S as ring members, wherein S-atoms as ring members may be present as S, SO or SO₂. Examples of 5- or 6-membered heterocyclic radicals comprise saturated or unsaturated, nonaromatic heterocyclic rings, such as 2- and 3-azetidinyl, 2- and 3-oxetanyl, 2- and 3-thietanyl, 2-and 3-thietanyl-S-oxid (S-oxothietanyl), 2- and 3-thietanyl-S-dioxid (S-dioxothietanyl), 2- and 3-pyrrolidinyl, 2- and 3-tetrahydrofuranyl, 1,3-dioxolan-2-yl, thiolan-2-yl, S-oxothiolan-2-yl, S-dioxothiolan-2-yl, 4- and 5-oxazolidinyl, 1,3-dioxan-2-yl, 1- and 3-thiopyran-2-yl, S-oxothiopyranyl, and S-dioxothiopyranyl.

The term “hetaryl” includes monocyclic 5- or 6-membered heteroaromatic radicals comprising as ring members 1, 2, or 3 heteroatoms selected from N, O and S. Examples of 5- or 6-membered heteroaromatic radicals include pyridyl, i.e. 2-, 3-, and 4-pyridyl, pyrimidinyl, i.e. 2-, 4- and 5-pyrimidinyl, pyrazinyl, pyridazinyl, i.e. 3- and 4-pyridazinyl, thienyl, i.e. 2- and 3-thienyl, furyl, i.e. 2- and 3-furyl, pyrrolyl, i.e. 1-, 2- and 3-pyrrolyl, oxazolyl, i.e. 2-, 4- and 5-oxazolyl, isoxazolyl, i.e. 3-, 4- and 5-isoxazolyl, thiazolyl, i.e. 2-, 3- and 5-thiazolyl, isothiazolyl, i.e. 3-, 4- and 5-isothiazolyl, pyrazolyl, i.e. 1-, 3-, 4- and 5-pyrazolyl, imidazolyl, i.e. 1-, 2-, 4- and 5-imidazolyl, oxadiazolyl, e.g. 2- and 5-[1,3,4]oxadiazolyl, thiadiazolyl, e.g. 1,3,4-thiadiazol-5-yl, 1,2,4-thiadiazol-3-yl, triazolyl, e.g. 1,3,4-triazol-2-yl, and 1,2,4-triazol-3-yl.

The terms “heterocyclyolalkyl” and “hetarylalkyl” refer to heterocyclyl or hetaryl, resp., as defined above which are bound via a C₁-C₄-alkyl group, in particular a methyl group (=heterocyclylmethyl or hetarylmethyl, resp.), to the remainder of the molecule.

With respect to the variables, the particularly preferred embodiments of the intermediates correspond to those of the compounds of the formula I.

In a particular embodiment, the variables of the compounds of the formula I have the following meanings, these meanings, both on their own and in combination with one another, being particular embodiments of the compounds of the formula I.

One embodiment relates to compounds of formula I wherein X is CH₂.

Another embodiment relates to compounds of formula I wherein X is S.

W-Z is preferably #-CH₂—O—*.

R¹ is preferably fluoromethyl, particularly CF₃.

R^{2a} is preferably selected from F, Cl, Br, CF₃, and OCF₃.

R²b and R^{2c} are independently preferably selected from H, F, Cl, Br, CF₃, and OCF₃.

Particularly preferred is each one of the following combinations of R^{2a}, R^{2b} and R^{2c} wherein each line of Table A denotes a substitution pattern of the phenyl ring (“A”) bearing the R^{2a}, R^{2b} and R^{2c} moieties.

TABLE A

| No. | R^{2a} | R^{2b} | R^{2c} |
|---|---|---|---|
| A-1 | F | F | H |
| A-2 | F | H | F |
| A-3 | F | F | F |
| A-4 | F | Cl | F |
| A-5 | F | Br | F |
| A-6 | F | H | Cl |
| A-7 | F | H | Br |
| A-8 | Cl | F | H |
| A-9 | Cl | H | Cl |
| A-10 | Cl | Cl | Cl |
| A-11 | Cl | F | Cl |
| A-12 | Cl | Br | Cl |
| A-13 | Cl | H | Br |
| A-14 | Br | F | H |
| A-15 | Br | H | Br |
| A-16 | Br | F | Br |
| A-17 | Br | Cl | Br |
| A-18 | CF₃ | H | H |
| A-19 | CF₃ | H | F |
| A-20 | CF₃ | H | Cl |
| A-21 | CF₃ | H | Br |
| A-22 | CF₃ | H | CF₃ |
| A-23 | CF₃ | F | F |
| A-24 | CF₃ | F | Cl |
| A-25 | CF₃ | Cl | Cl |
| A-26 | CF₃ | F | H |
| A-27 | OCF₃ | H | F |
| A-28 | OCF₃ | H | Cl |
| A-29 | OCF₃ | F | H |
| A-30 | OCF₃ | H | CF₃ |
| A-31 | OCF₃ | H | H |

Groups A-8, A-9, and A-11 are more preferred patterns in formula I compounds. A-11 is particularly preferred.

R³ is preferably selected from H, C₁-C₆-alkyl, C₁-C₆-haloalkyl, C₂-C₆-alkenyl, C₂-C₆-haloalkenyl, C₂-C₆-alkynyl, C₂-C₆-haloalkynyl which aliphatic groups are unsubstituted or substituted by one radical R³¹, C₃-C₆-cycloalkyl, C₃-C₆-halocycloalkyl which cyclic groups are unsubstituted or substituted by one radical R³²; heterocycle, or hetaryl which rings are unsubstituted or partially or fully substituted by R^{A};
R³¹ is independently OH, cyano, C₁-C₄-alkoxy, C₁-C₄-haloalkoxy, S(O)n—C₁-C₄-alkyl, S(O)ₙ—C₁-C₄-haloalkyl, C₃-C₅-cycloalkyl, or C₃-C₅-halocycloalkyl which cycles are unsubstituted or substituted by one or more R³¹¹;
   R³¹¹ is independently OH, cyano, C₁-C₂-alkyl, or C₁-C₂-haloalkyl;
   n is 0, 1, or 2;
R³² C₁-C₂-alkyl, C₁-C₂-haloalkyl, OH, CN;
R^{A} is independently selected from halogen, cyano, NO₂, C₁-C₂-alkyl, C₁-C₂-haloalkyl, C₂-C₃-alkenyl, C₂-C₃-haloalkenyl, C₂-C₃-alkynyl, C₂-C₃-haloalkynyl, C₃-C₅-cycloalkyl, C₃-C₅-halocycloalkyl, C₁-C₃-alkoxy, C₁-C₃-haloalkoxy, S(O)ₙ—C₁-C₃-alkyl, S(O)ₙ—C₁-C₃-haloalkyl; or two R^{A} present on the same carbon atom of a saturated or partially saturated ring may form together═O.

More preferably R³ is selected from C₁-C₄-alkyl, and C₁-C₄-haloalkyl, which aliphatic groups are unsubstituted or substituted by one radical R³¹; and C₃-C₅-cycloalkyl, C₃-C₅-halocycloalkyl which cyclic groups are unsubstituted or substituted by one radical R³²;
R³¹ is independently OH, cyano, C₁-C₄-alkoxy, C₁-C₄-haloalkoxy, S(O)ₙ—C₁-C₄-alkyl, S(O)n—C₁-C₄-haloalkyl, C₃-C₅-cycloalkyl, or C₃-C₅-halocycloalkyl which cycles are unsubstituted or substituted by one or more R³¹¹;
   R³¹¹ is independently OH, cyano, C₁-C₂-alkyl, or C₁-C₂-haloalkyl;
   n is 0, 1, or 2;
R³² C₁-C₂-alkyl, C₁-C₂-haloalkyl, OH, CN.

Particularly preferred R³ is selected from C₁-C₃-alkyl, CH₂CF₃, CH₂CH₂CF₃, CH₂OH, CH₂-c-C₃H₅; c-C₃H₅, 1-CN-c-C₃H₄, 1-CF₃-c-C₃H₄, 1-OH-c-C₃H₄, 2,2,-F₂-c-C₃H₃, CH₂-c-C₃H₅, CH₂OCH₃, CH₂OC₂H₅, CH₂OCF₃, CH₂OCH₂CF₃, and CH₂SOₙCH₃, CH₂SOₙC₂H₅, wherein n is 0, 1, or 2; 1-pyrazolyl, 3-CH₃-1-pyrazolyl, 2-pyridyl, 3-thietan-yl, 3-thietan-yl-S-oxide, and 3-thietan-yl-S-dioxide.

In another particularly preferred embodiment R³ is C₁-C₃-alkyl, particularly CH₃, C₂H₅, n-propyl; CH₂CF₃; C₂-C₃-alkenyl, particularly CH═CH₂, CH═CHCH₃; C₂-C₃-alkynyl, particularly C≡CH; c-C₃H₅, c-C₄H₇, 1-CF₃-c-C₃H₄, CH₂-c-C₃H₅; CH₂SOₙCH₃, CH₂SOₙC₂H₅, wherein n is 0, 1, or 2, and particularly n is 2; CH₂—C(═O)NHC₂H₅; CH₂-tetrahydrofuran-2-yl, CH₂-1,3-dioxolan-2-yl, CH₂-1,3-dioxan-2-yl, 1,3-dioxolan-2-yl, 2-oxetanyl, 3-thietanyl, CH₂-pyrimidin-2-yl, and 2-pyridyl.

Preferred embodiments relate to each of following compounds of formula I, wherein the variables are as defined in the outset and the preferred embodiments:

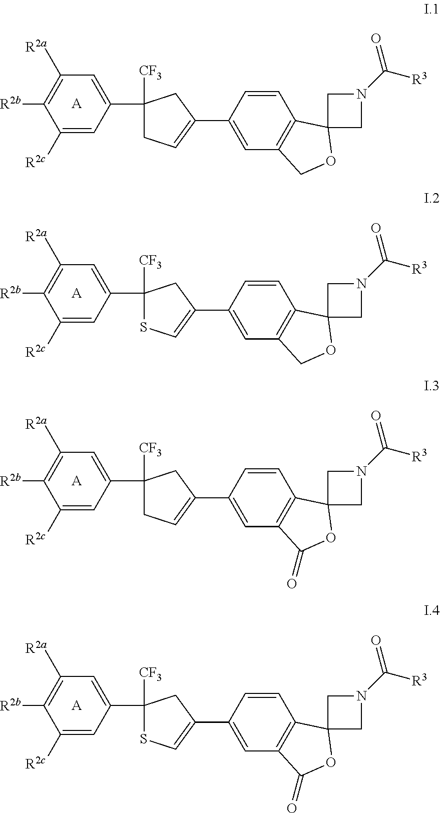

In particular with a view to their use, preference is given to the compounds of formula I compiled in the tables below, which compounds correspond to formulae I.1, I.2, I.3, and I.4, resp. Each of the groups mentioned for a substituent in the tables is furthermore per se, independently of the combination in which it is mentioned, a particularly preferred aspect of the substituent in question.

Table 1: Compounds of formula I.1 in which ring A is substituted by A-1 and R³ for a compound corresponds in each case to one row of Table B

Table 2: Compounds of formula I.2 in which ring A is substituted by A-1 and R³ for a compound corresponds in each case to one row of Table B

Table 3: Compounds of formula I.3 in which ring A is substituted by A-1 and R³ for a compound corresponds in each case to one row of Table B

Table 4: Compounds of formula I.4 in which ring A is substituted by A-1 and R³ for a compound corresponds in each case to one row of Table B

Table 5: Compounds of formula I.1 in which ring A is substituted by A-2 and R³ for a compound corresponds in each case to one row of Table B

Table 6: Compounds of formula I.2 in which ring A is substituted by A-2 and R³ for a compound corresponds in each case to one row of Table B

Table 7: Compounds of formula I.3 in which ring A is substituted by A-2 and R³ for a compound corresponds in each case to one row of Table B

Table 8: Compounds of formula I.4 in which ring A is substituted by A-2 and R³ for a compound corresponds in each case to one row of Table B

Table 9: Compounds of formula I.1 in which ring A is substituted by A-3 and R³ for a compound corresponds in each case to one row of Table B

Table 10: Compounds of formula I.2 in which ring A is substituted by A-3 and R³ for a compound corresponds in each case to one row of Table B

Table 11: Compounds of formula I.3 in which ring A is substituted by A-3 and R³ for a compound corresponds in each case to one row of Table B

Table 12: Compounds of formula I.4 in which ring A is substituted by A-3 and R³ for a compound corresponds in each case to one row of Table B

Table 13: Compounds of formula I.1 in which ring A is substituted by A-4 and R³ for a compound corresponds in each case to one row of Table B

Table 14: Compounds of formula I.2 in which ring A is substituted by A-4 and R³ for a compound corresponds in each case to one row of Table B

Table 15: Compounds of formula I.3 in which ring A is substituted by A-4 and R³ for a compound corresponds in each case to one row of Table B

Table 16: Compounds of formula I.4 in which ring A is substituted by A-4 and R³ for a compound corresponds in each case to one row of Table B

Table 17: Compounds of formula I.1 in which ring A is substituted by A-5 and R³ for a compound corresponds in each case to one row of Table B

Table 18: Compounds of formula I.2 in which ring A is substituted by A-5 and R³ for a compound corresponds in each case to one row of Table B

Table 19: Compounds of formula I.3 in which ring A is substituted by A-5 and R³ for a compound corresponds in each case to one row of Table B

Table 20: Compounds of formula I.4 in which ring A is substituted by A-5 and R³ for a compound corresponds in each case to one row of Table B

Table 21: Compounds of formula I.1 in which ring A is substituted by A-6 and R³ for a compound corresponds in each case to one row of Table B

Table 22: Compounds of formula I.2 in which ring A is substituted by A-6 and R³ for a compound corresponds in each case to one row of Table B

Table 23: Compounds of formula I.3 in which ring A is substituted by A-6 and R³ for a compound corresponds in each case to one row of Table B

Table 24: Compounds of formula I.4 in which ring A is substituted by A-6 and R³ for a compound corresponds in each case to one row of Table B

Table 25: Compounds of formula I.1 in which ring A is substituted by A-7 and R³ for a compound corresponds in each case to one row of Table B

Table 26: Compounds of formula I.2 in which ring A is substituted by A-7 and R³ for a compound corresponds in each case to one row of Table B

Table 27: Compounds of formula I.3 in which ring A is substituted by A-7 and R³ for a compound corresponds in each case to one row of Table B

Table 28: Compounds of formula I.4 in which ring A is substituted by A-7 and R³ for a compound corresponds in each case to one row of Table B

Table 29: Compounds of formula I.1 in which ring A is substituted by A-8 and R³ for a compound corresponds in each case to one row of Table B

Table 30: Compounds of formula I.2 in which ring A is substituted by A-8 and R³ for a compound corresponds in each case to one row of Table B

Table 31: Compounds of formula I.3 in which ring A is substituted by A-8 and R³ for a compound corresponds in each case to one row of Table B

Table 32: Compounds of formula I.4 in which ring A is substituted by A-8 and R³ for a compound corresponds in each case to one row of Table B

Table 33: Compounds of formula I.1 in which ring A is substituted by A-9 and R³ for a compound corresponds in each case to one row of Table B

Table 34: Compounds of formula I.2 in which ring A is substituted by A-9 and R³ for a compound corresponds in each case to one row of Table B

Table 35: Compounds of formula I.3 in which ring A is substituted by A-9 and R³ for a compound corresponds in each case to one row of Table B

Table 36: Compounds of formula I.4 in which ring A is substituted by A-9 and R³ for a compound corresponds in each case to one row of Table B

Table 37: Compounds of formula I.1 in which ring A is substituted by A-10 and R³ for a compound corresponds in each case to one row of Table B

Table 38: Compounds of formula I.2 in which ring A is substituted by A-10 and R³ for a compound corresponds in each case to one row of Table B

Table 39: Compounds of formula I.3 in which ring A is substituted by A-10 and R³ for a compound corresponds in each case to one row of Table B

Table 40: Compounds of formula I.4 in which ring A is substituted by A-10 and R³ for a compound corresponds in each case to one row of Table B

Table 41: Compounds of formula I.1 in which ring A is substituted by A-11 and R³ for a compound corresponds in each case to one row of Table B

Table 42: Compounds of formula I.2 in which ring A is substituted by A-11 and R³ for a compound corresponds in each case to one row of Table B

Table 43: Compounds of formula I.3 in which ring A is substituted by A-11 and R³ for a compound corresponds in each case to one row of Table B

Table 44: Compounds of formula I.4 in which ring A is substituted by A-11 and R³ for a compound corresponds in each case to one row of Table B

Table 45: Compounds of formula I.1 in which ring A is substituted by A-12 and R³ for a compound corresponds in each case to one row of Table B

Table 46: Compounds of formula I.2 in which ring A is substituted by A-12 and R³ for a compound corresponds in each case to one row of Table B

Table 47: Compounds of formula I.3 in which ring A is substituted by A-12 and R³ for a compound corresponds in each case to one row of Table B

Table 48: Compounds of formula I.4 in which ring A is substituted by A-12 and R³ for a compound corresponds in each case to one row of Table B

Table 49: Compounds of formula I.1 in which ring A is substituted by A-13 and R³ for a compound corresponds in each case to one row of Table B

Table 50: Compounds of formula I.2 in which ring A is substituted by A-13 and R³ for a compound corresponds in each case to one row of Table B

Table 51: Compounds of formula I.3 in which ring A is substituted by A-13 and R³ for a compound corresponds in each case to one row of Table B

Table 52: Compounds of formula I.4 in which ring A is substituted by A-13 and R³ for a compound corresponds in each case to one row of Table B

Table 53: Compounds of formula I.1 in which ring A is substituted by A-14 and R³ for a compound corresponds in each case to one row of Table B

Table 54: Compounds of formula I.2 in which ring A is substituted by A-14 and R³ for a compound corresponds in each case to one row of Table B

Table 55: Compounds of formula I.3 in which ring A is substituted by A-14 and R³ for a compound corresponds in each case to one row of Table B

Table 56: Compounds of formula I.4 in which ring A is substituted by A-14 and R³ for a compound corresponds in each case to one row of Table B

Table 57: Compounds of formula I.1 in which ring A is substituted by A-15 and R³ for a compound corresponds in each case to one row of Table B

Table 58: Compounds of formula I.2 in which ring A is substituted by A-15 and R³ for a compound corresponds in each case to one row of Table B

Table 59: Compounds of formula I.3 in which ring A is substituted by A-15 and R³ for a compound corresponds in each case to one row of Table B

Table 60: Compounds of formula I.4 in which ring A is substituted by A-15 and R³ for a compound corresponds in each case to one row of Table B

Table 61: Compounds of formula I.1 in which ring A is substituted by A-16 and R³ for a compound corresponds in each case to one row of Table B

Table 62: Compounds of formula I.2 in which ring A is substituted by A-16 and R³ for a compound corresponds in each case to one row of Table B

Table 63: Compounds of formula I.3 in which ring A is substituted by A-16 and R³ for a compound corresponds in each case to one row of Table B

Table 64: Compounds of formula I.4 in which ring A is substituted by A-16 and R³ for a compound corresponds in each case to one row of Table B

Table 65: Compounds of formula I.1 in which ring A is substituted by A-17 and R³ for a compound corresponds in each case to one row of Table B

Table 66: Compounds of formula I.2 in which ring A is substituted by A-17 and R³ for a compound corresponds in each case to one row of Table B

Table 67: Compounds of formula I.3 in which ring A is substituted by A-17 and R³ for a compound corresponds in each case to one row of Table B

Table 68: Compounds of formula I.4 in which ring A is substituted by A-17 and R³ for a compound corresponds in each case to one row of Table B

Table 69: Compounds of formula I.1 in which ring A is substituted by A-18 and R³ for a compound corresponds in each case to one row of Table B

Table 70: Compounds of formula I.2 in which ring A is substituted by A-18 and R³ for a compound corresponds in each case to one row of Table B

Table 71: Compounds of formula I.3 in which ring A is substituted by A-18 and R³ for a compound corresponds in each case to one row of Table B

Table 72: Compounds of formula I.4 in which ring A is substituted by A-18 and R³ for a compound corresponds in each case to one row of Table B

Table 73: Compounds of formula I.1 in which ring A is substituted by A-19 and R³ for a compound corresponds in each case to one row of Table B

Table 74: Compounds of formula I.2 in which ring A is substituted by A-19 and R³ for a compound corresponds in each case to one row of Table B

Table 75: Compounds of formula I.3 in which ring A is substituted by A-19 and R³ for a compound corresponds in each case to one row of Table B

Table 76: Compounds of formula I.4 in which ring A is substituted by A-19 and R³ for a compound corresponds in each case to one row of Table B

Table 77: Compounds of formula I.1 in which ring A is substituted by A-20 and R³ for a compound corresponds in each case to one row of Table B

Table 78: Compounds of formula I.2 in which ring A is substituted by A-20 and R³ for a compound corresponds in each case to one row of Table B

Table 79: Compounds of formula I.3 in which ring A is substituted by A-20 and R³ for a compound corresponds in each case to one row of Table B

Table 80: Compounds of formula I.4 in which ring A is substituted by A-20 and R³ for a compound corresponds in each case to one row of Table B

Table 81: Compounds of formula I.1 in which ring A is substituted by A-21 and R³ for a compound corresponds in each case to one row of Table B

Table 82: Compounds of formula I.2 in which ring A is substituted by A-21 and R³ for a compound corresponds in each case to one row of Table B

Table 83: Compounds of formula I.3 in which ring A is substituted by A-21 and R³ for a compound corresponds in each case to one row of Table B

Table 84: Compounds of formula I.4 in which ring A is substituted by A-21 and R³ for a compound corresponds in each case to one row of Table B

Table 85: Compounds of formula I.1 in which ring A is substituted by A-22 and R³ for a compound corresponds in each case to one row of Table B

Table 86: Compounds of formula I.2 in which ring A is substituted by A-22 and R³ for a compound corresponds in each case to one row of Table B

Table 87: Compounds of formula I.3 in which ring A is substituted by A-22 and R³ for a compound corresponds in each case to one row of Table B

Table 88: Compounds of formula I.4 in which ring A is substituted by A-22 and R³ for a compound corresponds in each case to one row of Table B

Table 89: Compounds of formula I.1 in which ring A is substituted by A-23 and R³ for a compound corresponds in each case to one row of Table B

Table 90: Compounds of formula I.2 in which ring A is substituted by A-23 and R³ for a compound corresponds in each case to one row of Table B

Table 91: Compounds of formula I.3 in which ring A is substituted by A-23 and R³ for a compound corresponds in each case to one row of Table B

Table 92: Compounds of formula I.4 in which ring A is substituted by A-23 and R³ for a compound corresponds in each case to one row of Table B

Table 93: Compounds of formula I.1 in which ring A is substituted by A-24 and R³ for a compound corresponds in each case to one row of Table B

Table 94: Compounds of formula I.2 in which ring A is substituted by A-24 and R³ for a compound corresponds in each case to one row of Table B

Table 95: Compounds of formula I.3 in which ring A is substituted by A-24 and R³ for a compound corresponds in each case to one row of Table B

Table 96: Compounds of formula I.4 in which ring A is substituted by A-24 and R³ for a compound corresponds in each case to one row of Table B

Table 97: Compounds of formula I.1 in which ring A is substituted by A-25 and R³ for a compound corresponds in each case to one row of Table B

Table 98: Compounds of formula I.2 in which ring A is substituted by A-25 and R³ for a compound corresponds in each case to one row of Table B

Table 99: Compounds of formula I.3 in which ring A is substituted by A-25 and R³ for a compound corresponds in each case to one row of Table B

Table 100: Compounds of formula I.4 in which ring A is substituted by A-25 and R³ for a compound corresponds in each case to one row of Table B

Table 101: Compounds of formula I.1 in which ring A is substituted by A-26 and R³ for a compound corresponds in each case to one row of Table B

Table 102: Compounds of formula I.2 in which ring A is substituted by A-26 and R³ for a compound corresponds in each case to one row of Table B

Table 103: Compounds of formula I.3 in which ring A is substituted by A-26 and R³ for a compound corresponds in each case to one row of Table B

Table 104: Compounds of formula I.4 in which ring A is substituted by A-26 and R³ for a compound corresponds in each case to one row of Table B

Table 105: Compounds of formula I.1 in which ring A is substituted by A-27 and R³ for a compound corresponds in each case to one row of Table B

Table 106: Compounds of formula I.2 in which ring A is substituted by A-27 and R³ for a compound corresponds in each case to one row of Table B

Table 107: Compounds of formula I.3 in which ring A is substituted by A-27 and R³ for a compound corresponds in each case to one row of Table B

Table 108: Compounds of formula I.4 in which ring A is substituted by A-27 and R³ for a compound corresponds in each case to one row of Table B

Table 109: Compounds of formula I.1 in which ring A is substituted by A-28 and R³ for a compound corresponds in each case to one row of Table B

Table 110: Compounds of formula I.2 in which ring A is substituted by A-28 and R³ for a compound corresponds in each case to one row of Table B

Table 111: Compounds of formula I.3 in which ring A is substituted by A-28 and R³ for a compound corresponds in each case to one row of Table B

Table 112: Compounds of formula I.4 in which ring A is substituted by A-28 and R³ for a compound corresponds in each case to one row of Table B

Table 113: Compounds of formula I.1 in which ring A is substituted by A-29 and R³ for a compound corresponds in each case to one row of Table B

Table 114: Compounds of formula I.2 in which ring A is substituted by A-29 and R³ for a compound corresponds in each case to one row of Table B

Table 115: Compounds of formula I.3 in which ring A is substituted by A-29 and R³ for a compound corresponds in each case to one row of Table B

Table 116: Compounds of formula I.4 in which ring A is substituted by A-29 and R³ for a compound corresponds in each case to one row of Table B

Table 117: Compounds of formula I.1 in which ring A is substituted by A-30 and R³ for a compound corresponds in each case to one row of Table B

Table 118: Compounds of formula I.2 in which ring A is substituted by A-30 and R³ for a compound corresponds in each case to one row of Table B

Table 119: Compounds of formula I.3 in which ring A is substituted by A-30 and R³ for a compound corresponds in each case to one row of Table B

Table 120: Compounds of formula I.4 in which ring A is substituted by A-30 and R³ for a compound corresponds in each case to one row of Table B

Table 121: Compounds of formula I.1 in which ring A is substituted by A-31 and R³ for a compound corresponds in each case to one row of Table B

Table 122: Compounds of formula I.2 in which ring A is substituted by A-31 and R³ for a compound corresponds in each case to one row of Table B

Table 123: Compounds of formula I.3 in which ring A is substituted by A-31 and R³ for a compound corresponds in each case to one row of Table B

Table 124: Compounds of formula I.4 in which ring A is substituted by A-31 and R³ for a compound corresponds in each case to one row of Table B

TABLE B

| No. | R³ |
|---|---|
| B-1 | H |
| B-2 | CH₃ |
| B-3 | CH₂CH₃ |
| B-4 | CH₂CH₂CH₃ |
| B-5 | CH(CH₃)₂ |
| B-6 | CH₂CH₂CH₂CH₃ |
| B-7 | CH(CH₃)CH₂CH₃ |
| B-8 | CH₂CH(CH₃)₂ |
| B-9 | C(CH₃)₃ |
| B-10 | CF₃ |
| B-11 | CH₂CF₃ |
| B-12 | CH₂CH₂CF₃ |
| B-13 | CH═CH₂ |
| B-14 | CH═CH—CH₃ |
| B-15 | CH₂—CH═CH₂ |
| B-16 | c-C₃H₅ |
| B-17 | 2,2-F₂—c-C₃H₃ |
| B-18 | 1-CF₃—c-C₃H₄ |
| B-19 | 1-CN-c-C₃H₄ |
| B-20 | c-C₄H₇ |
| B-21 | CH₂—c-C₃H₅ |
| B-22 | CH₂—c-C₄H₇ |
| B-23 | CH₂SCH₃ |
| B-24 | CH₂S(O)CH₃ |
| B-25 | CH₂SO₂CH₃ |
| B-26 | CH₂SCH₂CH₃ |
| B-27 | CH₂S(O)CH₂CH₃ |
| B-28 | CH₂SO₂CH₂CH₃ |
| B-29 | CH₂CH₂SCH₃ |
| B-30 | CH₂CH₂S(O)CH₃ |
| B-31 | CH₂CH₂SO₂CH₃ |
| B-32 | CH₂SCF₃ |
| B-33 | CH₂S(O)CF₃ |
| B-34 | CH₂SO₂CF₃ |
| B-35 | CH₂OCH₃ |
| B-36 | CH₂CH₂OCH₃ |
| B-37 | CH₂OCH₂CH₃ |
| B-38 | CH₂OCH₂CF₃ |
| B-39 | CH₂CH₂OCH₂CH₃ |
| B-40 | CH₂CH₂OCH₂CF₃ |
| B-41 | CH₂-(2-THF) |
| B-42 | CH₂-(1,3-DXL-2) |
| B-43 | CH₂-(1,3-DXN-2) |
| B-44 | CH₂-(OET-2) |
| B-45 | CH₂-(OET-3) |
| B-46 | CH₂-(PMD-2) |
| B-47 | CH₂-(PRD-2) |
| B-48 | CH₂-(PRZ-1) |
| B-49 | CH₂-(3-CH₃-PRZ-1) |
| B-50 | 2-PMD |
| B-51 | 2-PRD |
| B-52 | 1,3-DXL-2 |
| B-53 | 1,3-DXN-2 |
| B-54 | OET-2 |
| B-55 | OET-3 |
| B-56 | TET-3 |
| B-57 | 1-O-TET-3 |
| B-58 | 1,1-O₂-TET-3 |

Abbreviations:
THF = tetrahydrofuranyl
DXL = dioxolanyl
DXN = dioxanyl
OET = oxetanyl
PMD = pyrimidinyl
PRD = pyridinyl
PRZ = pyrazolyl
TET = thietanyl

The compounds of the present invention are suitable for use in protecting crops, plants, plant propagation materials, such as seeds, or soil or water, in which the plants are growing, from attack or infestation by animal pests. Therefore, the present invention also relates to a plant protection method, which comprises contacting crops, plants, plant propagation materials, such as seeds, or soil or water, in which the plants are growing, to be protected from attack or infestation by animal pests, with a pesticidally effective amount of a compound of the present invention.

The compounds of the present invention are also suitable for use in combating or controlling animal pests. Therefore, the present invention also relates to a method of combating or controlling animal pests, which comprises contacting the animal pests, their habitat, breeding ground, or food supply, or the crops, plants, plant propagation materials, such as seeds, or soil, or the area, material or environment in which the animal pests are growing or may grow, with a pesticidally effective amount of a compound of the present invention.

The compounds of the present invention are effective through both contact and ingestion. Furthermore, the compounds of the present invention can be applied to any and all developmental stages, such as egg, larva, pupa, and adult.

The compounds of the present invention can be applied as such or in form of compositions comprising them as defined above. Furthermore, the compounds of the present invention can be applied together with a mixing partner as defined above or in form of compositions comprising said mixtures as defined above. The components of said mixture can be applied simultaneously, jointly or separately, or in succession, that is immediately one after another and thereby creating the mixture “in situ” on the desired location, e.g. the plant, the sequence, in the case of separate application, generally not having any effect on the result of the control measures.

The application can be carried out both before and after the infestation of the crops, plants, plant propagation materials, such as seeds, soil, or the area, material or environment by the pests.

Suitable application methods include inter alia soil treatment, seed treatment, in furrow application, and foliar application. Soil treatment methods include drenching the soil, drip irrigation (drip application onto the soil), dipping roots, tubers or bulbs, or soil injection. Seed treatment techniques include seed dressing, seed coating, seed dusting, seed soaking, and seed pelleting. In furrow applications typically include the steps of making a furrow in cultivated land, seeding the furrow with seeds, applying the pesticidally active compound to the furrow, and closing the furrow. Foliar application refers to the application of the pesticidally active compound to plant foliage, e.g. through spray equipment. For foliar applications, it can be advantageous to modify the behavior of the pests by use of pheromones in combination with the compounds of the present invention. Suitable pheromones for specific crops and pests are known to a skilled person and publicly available from databases of pheromones and semiochemicals, such as http://www.pherobase.com.

As used herein, the term “contacting” includes both direct contact (applying the compounds/compositions directly on the animal pest or plant—typically to the foliage, stem or roots of the plant) and indirect contact (applying the compounds/compositions to the locus, i.e. habitat, breeding ground, plant, seed, soil, area, material or environment in which a pest is growing or may grow, of the animal pest or plant).

The term “animal pest” includes arthropods, gastropods, and nematodes. Preferred animal pests according to the invention are arthropods, preferably insects and arachnids, in particular insects. Insects, which are of particular relevance for crops, are typically referred to as crop insect pests.

The term “crop” refers to both, growing and harvested crops.

The term “plant” includes cereals, e.g. durum and other wheat, rye, barley, triticale, oats, rice, or maize (fodder maize and sugar maize/sweet and field corn); beet, e.g. sugar beet or fodder beet; fruits, such as pomes, stone fruits or soft fruits, e.g. apples, pears, plums, peaches, nectarines, almonds, cherries, papayas, strawberries, raspberries, blackberries or gooseberries; leguminous plants, such as beans, lentils, peas, alfalfa or soybeans; oil plants, such as rapeseed (oilseed rape), turnip rape, mustard, olives, sunflowers, coconut, cocoa beans, castor oil plants, oil palms, ground nuts or soybeans; cucurbits, such as squashes, pumpkins, cucumber or melons; fiber plants, such as cotton, flax, hemp or jute; citrus fruit, such as oranges, lemons, grapefruits or mandarins; vegetables, such as eggplant, spinach, lettuce (e.g. iceberg lettuce), chicory, cabbage, asparagus, cabbages, carrots, onions, garlic, leeks, tomatoes, potatoes, cucurbits or sweet peppers; lauraceous plants, such as avocados, cinnamon or camphor; energy and raw material plants, such as corn, soybean, rapeseed, sugar cane or oil palm; tobacco; nuts, e.g. walnuts; pistachios; coffee; tea; bananas; vines (table grapes and grape juice grape vines); hop; sweet leaf (also called Stevia); natural rubber plants or ornamental and forestry plants, such as flowers (e.g. carnation, petunias, geranium/pelargoniums, pansies and impatiens), shrubs, broad-leaved trees (e.g. poplar) or evergreens, e.g. conifers; eucalyptus; turf; lawn; grass such as grass for animal feed or ornamental uses. Preferred plants include potatoes sugar beets, tobacco, wheat, rye, barley, oats, rice, corn, cotton, soybeans, rapeseed, legumes, sunflowers, coffee or sugar cane; fruits; vines; ornamentals; or vegetables, such as cucumbers, tomatoes, beans or squashes.

The term “plant” is to be understood as including wild type plants and plants, which have been modified by either conventional breeding, or mutagenesis or genetic engineering, or by a combination thereof.

Plants, which have been modified by mutagenesis or genetic engineering, and are of particular commercial importance, include alfalfa, rapeseed (e.g. oilseed rape), bean, carnation, chicory, cotton, eggplant, eucalyptus, flax, lentil, maize, melon, papaya, petunia, plum, poplar, potato, rice, soybean, squash, sugar beet, sugarcane, sunflower, sweet pepper, tobacco, tomato, and cereals (e.g. wheat), in particular maize, soybean, cotton, wheat, and rice. In plants, which have been modified by mutagenesis or genetic engineering, one or more genes have been mutagenized or integrated into the genetic material of the plant. The one or more mutagenized or integrated genes are preferably selected from pat, epsps, cry1Ab, bar, cry1Fa2, cry1Ac, cry34Ab1, cry35AB1, cry3A, cryF, cry1F, mcry3a, cry2Ab2, cry3Bb1, cry1A.105, dfr, barnase, vip3Aa20, barstar, als, bxn, bp40, asn1, and ppo5. The mutagenesis or integration of the one or more genes is performed in order to improve certain properties of the plant. Such properties, also known as traits, include abiotic stress tolerance, altered growth/yield, disease resistance, herbicide tolerance, insect resistance, modified product quality, and pollination control. Of these properties, herbicide tolerance, e.g. imidazolinone tolerance, glyphosate tolerance, or glufosinate tolerance, is of particular importance. Several plants have been rendered tolerant to herbicides by mutagenesis, for example Clearfield® oilseed rape being tolerant to imidazolinones, e.g. imazamox. Alternatively, genetic engineering methods have been used to render plants, such as soybean, cotton, corn, beets and oil seed rape, tolerant to herbicides, such as glyphosate and glufosinate, some of which are commercially available under the trade names RoundupReady® (glyphosate) and LibertyLink® (glufosinate). Furthermore, insect resistance is of importance, in particular lepidopteran insect resistance and coleopteran insect resistance. Insect resistance is typically achieved by modifying plants by integrating cry and/or vip genes, which were isolated from Bacillus thuringiensis (Bt), and code for the respective Bt toxins. Genetically modified plants with insect resistance are commercially available under trade names including WideStrike®, Bollgard®, Agrisure®, Herculex®, YieldGard®, Genuity®, and Intacta®. Plants may be modified by mutagenesis or genetic engineering either in terms of one property (singular traits) or in terms of a combination of properties (stacked traits). Stacked traits, e.g. the combination of herbicide tolerance and insect resistance, are of increasing importance. In general, all relevant modified plants in connection with singular or stacked traits as well as detailed information as to the mutagenized or integrated genes and the respective events are available from websites of the organizations “International Service for the Acquisition of Agri-biotech Applications (ISAAA)” (http://www.isaaa.org/gmapprovaldatabase) and “Center for Environmental Risk Assessment (CERA)” (http://cera-gmc.org/GMCropDatabase).

It has surprisingly been found that the pesticidal activity of the compounds of the present invention may be enhanced by the insecticidal trait of a modified plant. Furthermore, it has been found that the compounds of the present invention are suitable for preventing insects to become resistant to the insecticidal trait or for combating pests, which already have become resistant to the insecticidal trait of a modified plant. Moreover, the compounds of the present invention are suitable for combating pests, against which the insecticidal trait is not effective, so that a complementary insecticidal activity can advantageously be used.

The term “plant propagation material” refers to all the generative parts of the plant such as seeds and vegetative plant material such as cuttings and tubers (e.g. potatoes), which can be used for the multiplication of the plant. This includes seeds, roots, fruits, tubers, bulbs, rhizomes, shoots, sprouts and other parts of plants. Seedlings and young plants, which are to be transplanted after germination or after emergence from soil, may also be included. These plant propagation materials may be treated prophylactically with a plant protection compound either at or before planting or transplanting.

The term “seed” embraces seeds and plant propagules of all kinds including but not limited to true seeds, seed pieces, suckers, corms, bulbs, fruit, tubers, grains, cuttings, cut shoots and the like, and means in a preferred embodiment true seeds.

In general, “pesticidally effective amount” means the amount of active ingredient needed to achieve an observable effect on growth, including the effects of necrosis, death, retardation, prevention, and removal, destruction, or otherwise diminishing the occurrence and activity of the target organism. The pesticidally effective amount can vary for the various compounds/compositions used in the invention. A pesticidally effective amount of the compositions will also vary according to the prevailing conditions such as desired pesticidal effect and duration, weather, target species, locus, mode of application, and the like.

In the case of soil treatment, in furrow application or of application to the pests dwelling place or nest, the quantity of active ingredient ranges from 0.0001 to 500 g per 100 m², preferably from 0.001 to 20 g per 100 m².

For use in treating crop plants, e.g. by foliar application, the rate of application of the active ingredients of this invention may be in the range of 0.0001 g to 4000 g per hectare, e.g. from 1 g to 2 kg per hectare or from 1 g to 750 g per hectare, desirably from 1 g to 100 g per hectare, more desirably from 10 g to 50 g per hectare, e.g., 10 to 20 g per hectare, 20 to 30 g per hectare, 30 to 40 g per hectare, or 40 to 50 g per hectare.

The compounds of the present invention are particularly suitable for use in the treatment of seeds in order to protect the seeds from insect pests, in particular from soil-living insect pests, and the resulting seedling's roots and shoots against soil pests and foliar insects. The present invention therefore also relates to a method for the protection of seeds from insects, in particular from soil insects, and of the seedling's roots and shoots from insects, in particular from soil and foliar insects, said method comprising treating the seeds before sowing and/or after pregermination with a compound of the present invention. The protection of the seedling's roots and shoots is preferred. More preferred is the protection of seedling's shoots from piercing and sucking insects, chewing insects and nematodes.

The term “seed treatment” comprises all suitable seed treatment techniques known in the art, such as seed dressing, seed coating, seed dusting, seed soaking, seed pelleting, and in-furrow application methods. Preferably, the seed treatment application of the active compound is carried out by spraying or by dusting the seeds before sowing of the plants and before emergence of the plants.

The present invention also comprises seeds coated with or containing the active compound. The term “coated with and/or containing” generally signifies that the active ingredient is for the most part on the surface of the propagation product at the time of application, although a greater or lesser part of the ingredient may penetrate into the propagation product, depending on the method of application. When the said propagation product is (re)planted, it may absorb the active ingredient.

Suitable seed is for example seed of cereals, root crops, oil crops, vegetables, spices, ornamentals, for example seed of durum and other wheat, barley, oats, rye, maize (fodder maize and sugar maize/sweet and field corn), soybeans, oil crops, crucifers, cotton, sunflowers, bananas, rice, oilseed rape, turnip rape, sugarbeet, fodder beet, eggplants, potatoes, grass, lawn, turf, fodder grass, tomatoes, leeks, pumpkin/squash, cabbage, iceberg lettuce, pepper, cucumbers, melons, Brassica species, melons, beans, peas, garlic, onions, carrots, tuberous plants such as potatoes, sugar cane, tobacco, grapes, petunias, geranium/pelargoniums, pansies and impatiens.

In addition, the active compound may also be used for the treatment of seeds from plants, which have been modified by mutagenisis or genetic engineering, and which e.g. tolerate the action of herbicides or fungicides or insecticides. Such modified plants have been described in detail above.

Conventional seed treatment formulations include for example flowable concentrates FS, solutions LS, suspoemulsions (SE), powders for dry treatment DS, water dispersible powders for slurry treatment WS, water-soluble powders SS and emulsion ES and EC and gel formulation GF. These formulations can be applied to the seed diluted or undiluted. Application to the seeds is carried out before sowing, either directly on the seeds or after having pregerminated the latter. Preferably, the formulations are applied such that germination is not included.

The active substance concentrations in ready-to-use formulations, which may be obtained after two-to-tenfold dilution, are preferably from 0.01 to 60% by weight, more preferably from 0.1 to 40% by weight.

In a preferred embodiment a FS formulation is used for seed treatment. Typically, a FS formulation may comprise 1-800 g/l of active ingredient, 1-200 g/l Surfactant, 0 to 200 g/l antifreezing agent, 0 to 400 g/l of binder, 0 to 200 g/l of a pigment and up to 1 liter of a solvent, preferably water.

Especially preferred FS formulations of the compounds of the present invention for seed treatment usually comprise from 0.1 to 80% by weight (1 to 800 g/l) of the active ingredient, from 0.1 to 20% by weight (1 to 200 g/l) of at least one surfactant, e.g. 0.05 to 5% by weight of a wetter and from 0.5 to 15% by weight of a dispersing agent, up to 20% by weight, e.g. from 5 to 20% of an anti-freeze agent, from 0 to 15% by weight, e.g. 1 to 15% by weight of a pigment and/or a dye, from 0 to 40% by weight, e.g. 1 to 40% by weight of a binder (sticker/adhesion agent), optionally up to 5% by weight, e.g. from 0.1 to 5% by weight of a thickener, optionally from 0.1 to 2% of an anti-foam agent, and optionally a preservative such as a biocide, antioxidant or the like, e.g. in an amount from 0.01 to 1% by weight and a filler/vehicle up to 100% by weight.

In the treatment of seed, the application rates of the compounds of the invention are generally from 0.1 g to 10 kg per 100 kg of seed, preferably from 1 g to 5 kg per 100 kg of seed, more preferably from 1 g to 1000 g per 100 kg of seed and in particular from 1 g to 200 g per 100 kg of seed, e.g. from 1 g to 100 g or from 5 g to 100 g per 100 kg of seed.

The invention therefore also relates to seed comprising a compound of the present invention, or an agriculturally useful salt thereof, as defined herein. The amount of the compound of the present invention or the agriculturally useful salt thereof will in general vary from 0.1 g to 10 kg per 100 kg of seed, preferably from 1 g to 5 kg per 100 kg of seed, in particular from 1 g to 1000 g per 100 kg of seed. For specific crops such as lettuce the rate can be higher.

The compounds of the present invention may also be used for improving the health of a plant. Therefore, the present invention also relates to a method for improving plant health by treating a plant, plant propagation material and/or the locus where the plant is growing or is to grow with an effective and non-phytotoxic amount of a compound of the present invention.

As used herein “an effective and non-phytotoxic amount” means that the compound is used in a quantity which allows to obtain the desired effect but which does not give rise to any phytotoxic symptom on the treated plant or on the plant grown from the treated propagule or treated soil.

The terms “plant” and “plant propagation material” are defined above. “Plant health” is defined as a condition of the plant and/or its products which is determined by several aspects alone or in combination with each other such as yield (for example increased biomass and/or increased content of valuable ingredients), quality (for example improved content or composition of certain ingredients or shelf life), plant vigour (for example improved plant growth and/or greener leaves (“greening effect”), tolerance to abiotic (for example drought) and/or biotic stress (for example disease) and production efficiency (for example, harvesting efficiency, processability).

The above identified indicators for the health condition of a plant may be interdependent and may result from each other. Each indicator is defined in the art and can be determined by methods known to a skilled person.

The compounds of the invention are also suitable for use against non-crop insect pests. For use against said non-crop pests, compounds of the present invention can be used as bait composition, gel, general insect spray, aerosol, as ultra-low volume application and bed net (impregnated or surface applied). Furthermore, drenching and rodding methods can be used.

As used herein, the term “non-crop insect pest” refers to pests, which are particularly relevant for non-crop targets, such as ants, termites, wasps, flies, ticks, mosquitos, crickets, or cockroaches.

The bait can be a liquid, a solid or a semisolid preparation (e.g. a gel). The bait employed in the composition is a product, which is sufficiently attractive to incite insects such as ants, termites, wasps, flies, mosquitos, crickets etc. or cockroaches to eat it. The attractiveness can be manipulated by using feeding stimulants or sex pheromones. Food stimulants are chosen, for example, but not exclusively, from animal and/or plant proteins (meat-, fish- or blood meal, insect parts, egg yolk), from fats and oils of animal and/or plant origin, or mono-, oligo- or polyorganosaccharides, especially from sucrose, lactose, fructose, dextrose, glucose, starch, pectin or even molasses or honey. Fresh or decaying parts of fruits, crops, plants, animals, insects or specific parts thereof can also serve as a feeding stimulant. Sex pheromones are known to be more insect specific. Specific pheromones are described in the literature (e.g. http://www.pherobase.com), and are known to those skilled in the art.

For use in bait compositions, the typical content of active ingredient is from 0.001 weight % to 15 weight %, desirably from 0.001 weight % to 5% weight % of active compound.

Formulations of the compounds of the present invention as aerosols (e.g in spray cans), oil sprays or pump sprays are highly suitable for the non-professional user for controlling pests such as flies, fleas, ticks, mosquitos or cockroaches. Aerosol recipes are preferably composed of the active compound, solvents, furthermore auxiliaries such as emulsifiers, perfume oils, if appropriate stabilizers, and, if required, propellants.

The oil spray formulations differ from the aerosol recipes in that no propellants are used.

For use in spray compositions, the content of active ingredient is from 0.001 to 80 weights %, preferably from 0.01 to 50 weight % and most preferably from 0.01 to 15 weight %.

The compounds of the present invention and its respective compositions can also be used in mosquito and fumigating coils, smoke cartridges, vaporizer plates or long-term vaporizers and also in moth papers, moth pads or other heat-independent vaporizer systems.

Methods to control infectious diseases transmitted by insects (e.g. malaria, dengue and yellow fever, lymphatic filariasis, and leishmaniasis) with compounds of the present invention and its respective compositions also comprise treating surfaces of huts and houses, air spraying and impregnation of curtains, tents, clothing items, bed nets, tsetse-fly trap or the like. Insecticidal compositions for application to fibers, fabric, knitgoods, nonwovens, netting material or foils and tarpaulins preferably comprise a mixture including the insecticide, optionally a repellent and at least one binder.

The compounds of the present invention and its compositions can be used for protecting wooden materials such as trees, board fences, sleepers, frames, artistic artifacts, etc. and buildings, but also construction materials, furniture, leathers, fibers, vinyl articles, electric wires and cables etc. from ants and/or termites, and for controlling ants and termites from doing harm to crops or human being (e.g. when the pests invade into houses and public facilities).

Customary application rates in the protection of materials are, for example, from 0.001 g to 2000 g or from 0.01 g to 1000 g of active compound per m² treated material, desirably from 0.1 g to 50 g per m².

Insecticidal compositions for use in the impregnation of materials typically contain from 0.001 to 95 weight %, preferably from 0.1 to 45 weight %, and more preferably from 1 to 25 weight % of at least one repellent and/or insecticide.

The compounds of the the present invention are especially suitable for efficiently combating animal pests such as arthropods, gastropods and nematodes including but not limited to:

insects from the order of Lepidoptera, for example Achroia grisella, Acleris spp. such as A. fimbriana, A. gloverana, A. variana; Acrolepiopsis assectella, Acronicta major, Adoxophyes spp. such as A. cyrtosema, A. orana; Aedia leucomelas, Agrotis spp. such as A. exclamationis, A. fucosa, A. ipsilon, A. orthogoma, A. segetum, A. subterranea; Alabama argillacea, Aleurodicus dispersus, Alsophila pometaria, Ampelophaga rubiginosa, Amyelois transitella, Anacampsis sarcitella, Anagasta kuehniella, Anarsia lineatella, Anisota senatoria, Antheraea pernyi, Anticarsia(=Thermesia) spp. such as A. gemmatalis, Apamea spp., Aproaerema modicella, Archips spp. such as A. argyrospila, A. fuscocupreanus, A. rosana, A. xyloseanus; Argyresthia conjugella, Argyroploce spp., Argyrotaenia spp. such as A. velutinana; Athetis mindara, Austroasca viridigrisea, Autographa gamma, Autographa nigrisigna, Barathra brassicae, Bedellia spp., Bonagota salubricola, Borbo cinnara, Bucculatrix thurberiella, Bupalus piniarius, Busseola spp., Cacoecia spp. such as C. murinana, C. podana; Cactoblasnis cactorum, Cadra cautella, Calingo braziliensis, Caloptilis theivora, Capua reticulana, Carposina spp. such as C. niponensis, C. sasakii; Cephus spp., Chaetocnema aridula, Cheimatobia brumata, Chilo spp. such as C. Indicus, C. suppressalis, C. partellus; Choreutis pariana, Choristoneura spp. such as C. conflictana, C. fumiferana, C. longicellana, C. murinana, C. occidentalis, C. rosaceana; Chlysodeixis (=Pseudoplusia)spp. such as C. eriosoma, C. includens; Cirphis unipuncta, Clysia ambiguella, Cnaphalocerus spp., Cnaphalocrocis medinalis, Cnephasia spp., Cochyils hospes, Coleophora spp., Colias eurytheme, Conopomorpha spp., Conotrachelus spp., Copitarsia spp., Corcyra cephalonica, Crambus caliginosellus, Crambus teterrellus, Crocidosema(=Epinotia) aporema, Cydalima (=Diaphania) perspectalis, Cydia (=Carpocapsa) spp. such as C. pomonella, C. latiferreana; Dalaca noctuides, Datana integerrima, Dasychira pinicola, Dendrolimus spp. such as D. pini, D. spectabilis, D. sibiricus; Desmia funeralis, Diaphania spp. such as D. nitidalis, D. hyalinata; Datraea grandiosella, Diatraea saccharalis, Diphthera festiva, Earias spp. such as E. insulana, E. vittella; Ecdytolopha aurantianu, Egira (=Xylomyges) curialis, Elasmopalpus lignosellus, Eldana saccharina, Endopiza viteana, Ennomos subsignaria, Eoreuma loftini, Ephestia spp. such as E. cautella, E. elutella, E. kuehniella; Epinotia aporema, Epiphyas postvittana, Erannis tiliaria, Erionota thrax, Etiella spp., Eulia spp., Eupoecilia ambiguella, Euproctis chrysorrhoea, Euxoa spp., Evetria bouliana, Faronta albilinea, Feltia spp. such as F. subterranean; Galleria mellonella, Gracillaria spp., Grapholita spp. such as G. funebrana, G. molesta, G. inopinata; Halysidota spp., Harrisina americana, Hedylepta spp., Helicoverpa spp. such as H. armigera (=Heliothis armigera), H. zea (=Heliothis zea); Heliothis spp. such as H. assulta, H. subflexa, H. virescens; Hellula spp. such as H. undalis, H. rogatalis; Helocoverpa gelotopoeon, Hemileuca oliviae, Herpetogramma licarsisalis, Hibernia defoliaria, Hofmannophila pseudospretella, Homoeosoma electellum, Homona magnanima, Hypena scabra, Hyphantria cunea, Hyponomeuta padella, Hyponomeuta malinellus, Kakivoria flavofasciata, Keiferia lycopersicella, Lambdina fiscellaria fiscellaria, Lambdina fiscellaria lugubrosa, Lamprosema indicata, Laspeyresia molesta, Leguminivora glycinivorella, Lerodea eufala, Leucinodes orbonalis, Leucoma salicis, Leucoptera spp. such as L. coffeella, L. scitella; Leuminivora lycinivorella, Lithocolletis blancardella, Lithophane antennata, Llattia octo (=Amyna axis), Lobesia botrana, Lophocampa spp., Loxagrotis albicosta, Loxostege spp. such as L. sticticalis, L. cereralis; Lymantria spp. such as L. dispar, L. monacha; Lyonetia clerkella, Lyonetia prunifoliella, Malacosoma spp. such as M. americanum, M. californicum, M. constrictum, M. neustria; Mamestra spp. such as M. brassicae, M. configurata; Mamstra brassicae, Manduca spp. such as M. quinquemaculata, M. sexta; Marasmia spp, Marmara spp., Maruca testulalis, Megalopyge lanata, Melanchra picta, Melanins leda, Mocis spp. such as M. lapites, M. repanda; Mocis latipes, Monochroa fragariae, Mythimna separata, Nemapogon cloacella, Neoleucinodes elegantalis, Nepytia spp., Nymphula spp., Oiketicus spp., Omiodes indicata, Omphisa anastomosalis, Operophtera brumata, Orgyia pseudotsugata, Oria spp., Orthaga thyrisalis, Ostrinia spp. such as O. nubilalis; Oulema oryzae, Paleacrita vernata, Panoils flammea, Parnara spp., Papaipema nebris, Papilio cresphontes, Paramyelois transitella, Paranthrene regalis, Paysandisia archon, Pectinophora spp. such as P. gossypiella; Peridroma saucia, Perileucoptera spp., such as P. coffeella; Phalera bucephala, Phryganidia californica, Phthorimaea spp. such as P. operculella; Phyllocnistis citrella, Phyllonorycter spp. such as P. blancardella, P. crataegella, P. issikii P. ringoniella; Pieris spp. such as P. brassicae, P. rapae, P. napi; Pilocroclis tripunctata, Plathypena scabra, Platynota spp. such as P. flavedana, P. idaeusalis, P. stultana; Platyptilia carduidactyla, Plebejus argus, Plodia interpunctella, Plusia spp, Plutella maculipennis, Plutella xylostella, Pontia protodica, Prays spp., Prodenia spp., Proxenus lepigone, Pseudaletia spp. such as P. sequax, P. unipuncta; Pyrausta nubilasis, Rachiplusia nu, Richia albicosta, Rhizobius ventralis, Rhyacionia frustrana, Sabulodes aegrotata, Schizura concinna, Schoenobius spp., Schreckensteinia festaliella, Scirpophaga spp. such as S. incertulas, S. innotata; Scotia segetum, Sesamia spp. such as S. inferens, Seudyra subflava, Sitotroga cerealella, Sparganothis pilleriana, Spilonota lechriaspis, S. ocellana, Spodoptera (=Lamphygma) spp. such as S. cosmoides, S. eridania, S. exigua, S. frugiperda, S. latisfascia, S. littoralis, S. litura, S. omithogalli; Stigmella spp., Stomopteryx subsecivella, Strymon bazochii, Sylepta derogata, Synanthedon spp. such as S. exitiosa, Tecia solanivora, Telehin Thaumetopoea pityocampa, Thaumatotibia (=Cryptophlebia) leucotreta, Thaumetopoea pityocampa, Thecla spp., Theresimima ampelophaga, Thyrinteina spp, Tildenia inconspicuella, Tinea spp. such as T. cloacella, T. pellionella; Tineola bisselliella, Tortrix spp. such as T. viridana; Trichophaga tapetzella, Trichoplusia spp. such as T. ni; Tuta (=Scrobipalpula) absoluta, Udea spp. such as U. rubigalis, U. rubigalis; Virachola spp., Yponomeuta padella, and Zeiraphera canadensis;

insects from the order of Coleoptera, for example Acalymma vittatum, Acanthoscehdes obtectus, Adoretus spp., Agelastica alni, Agrilus spp. such as A. anxius, A. planipennis, A. sinuatus; Agriotes spp. such as A. fuscicollis, A. lineatus, A. obscurus; Alphitobius diaperinus, Amphimallus solstitialis, Anisandrus dispar, Anisoplia austriaca, Anobium punctatum, Anomala corpulenta, Anomala rufocuprea, Anoplophora spp. such as A. glabripennis; Anthonomus spp. such as A. eugenii, A. grandis, A. pomorum; Anthrenus spp., Aphthona euphoridae, Apion spp., Apogonia spp., Athous haemorrhoidalis, Atomaria spp. such as A. linearis; Attagenus spp., Aulacophora femoralis, Blastophagus piniperda, Blitophaga undata, Bruchidius obtectus, Bruchus spp. such as B. lentis, B. pisorum, B. rufimanus; Byctiscus betulae, Callidiellum rufipenne, Cappopsistria floridensis, Callosobruchus chinensis, Cameraria ohridella, Cassida nebulosa, Cerotoma trifurcata, Cetonia aurata, Ceuthorhynchus spp. such as C. assimilis, C. napi; Chaetocnema tibialis Cleonus mendicus, Conoderus spp. such as C. vespertinus; Conotrachelus nenuphar, Cosmopolites spp., Costelytra zealandica, Crioceris asparagi, Cryptolestes ferrugineus, Cryptorhynchus lapathi, Ctenicera spp. such as C. destructor; Curculio spp., Cylindrocopturus spp., Cyclocephala spp., Dactylispa balyi, Dectes texanus, Dermestes spp., Diabrotica spp. such as D. undecimpunctata, D. speciosa, D. longicornis, D. semipunctata, D. virgifera; Diaprepes abbreviates, Dichocrocis spp., Dicladispa armigera, Diloboderus abderus, Diocalandra frumenti (Diocalandra stigmaticollis), Enaphalodes rufulus, Epilachna spp. such as E. varivestis, E. vigintioctomaculata; Epitrix spp. such as E. hirtipennis, E. simllaris; Eutheola humilis, Eutinobothrus brasiliensis, Faustinus cubae, Gibbium psylloides, Gnathocerus cornutus, Hellula undalis, Heteronychus arator, Hylamorpha elegans, Hylobius abietis, Hylotrupes bajulus, Hypera spp. such as H. brunneipennis, H. postica, Hypomeces squamosus, Hypothenemus spp., Ips typographus, Lachnosterna consanguinea, Lasioderma serricome, Latheticus oryzae, Lathridius spp., Lema spp. such as L. bilineata, L. melanopus; Leptinotarsa spp. such as L. decemlineata; Leptispa pygmaea, Limonius californicus, Lissorhoptrus oryzophilus, Lixus spp., Luperodes spp., Lyctus spp. such as L. bruneus; Liogenys fuscus, Macrodactylus spp. such as M. subspinosus; Maladera matrida, Megaplatypus mutates, Megascelis spp., Melanotus communis, Meligethes spp. such as M. aeneus, Melolontha spp. such as M. hippocastani, M. melolontha; Metamasius hemipterus, Microtheca spp., Migdolus spp. such as M. fryanus, Monochamus spp. such as M. alternatus; Naupactus xanthographus, Niptus hololeucus, Oberia brevis, Oemona hirta, Oryctes rhinoceros, Oryzaephilus surinamensis, Oryzaphagus oryzae, Otiorrhynchus sulcatus, Otiorrhynchus ovatus, Otiorrhynchus sulcatus, Oulema melanopus, Oulema oryzae, Oxycetonia jucunda, Phaedon spp. such as P. brassicae, P. cochleariae; Phoracantha recurva, Phyllobius pyri, Phyllopertha horticola, Phyllophaga spp. such as P. helleri; Phyllotreta spp. such as P. chrysocephala, P. nemorum, P. striolata, P. vittula; Phyllopertha horticola, Popillia japonica, Premnotrypes spp., Psacothea hilaris, Psylliodes chrysocephala, Prostephanus truncates, Psylliodes spp., Ptinus spp., Pulga saltona, Rhizopertha dominica, Rhynchophorus spp. such as R. billineatus, R. ferrugineus, R. palmarum, R. phoenicis, R. vulneratus; Saperda candida, Scolytus schevyrewi, Scyphophorus acupunctatus, Sitona lineatus, Sitophllus spp. such as S. granaria, S. oryzae, S. zeamais; Sphenophorus spp. such as S. Levis; Stegobium paniceum, Sternechus spp. such as S. subsignatus; Strophomorphus ctenotus, Symphyletes spp., Tanymecus spp., Tenebrio molitor, Tenebrioides mauretanicus, Tribolium spp. such as T. castaneum; Trogoderma spp., Tychius spp., Xylotrechus spp. such as X. pyrrhoderus; and, Zabrus spp. such as Z tenebrioides;

insects from the order of Diptera for example Aedes spp. such as A. aegypti, A. albopictus, A. vexans; Anastrepha ludens, Anopheles spp. such as A. albimanus, A. crucians, A. freeborni, A. gambiae, A. leucosphyrus, A. maculipennis, A. minimus, A. quadrimaculatus, A. sinensis; Bactrocera invadens, Bibio hortulanus, Calliphora erythrocephala, Calliphora vicina, Ceratitis capitata, Chrysomyia spp. such as C. bezziana, C. hominivorax, C. macellaria; Chrysops atlanticus, Chrysops discalis, Chrysops silacea, Cochliomyia spp. such as C. hominivorax; Contarinia spp. such as C. sorghicola; Cordylobia anthropophaga, Culex spp. such as C. nignpalpus, C. pipiens, C. quinquefasciatus, C. tarsalis, C. tritaeniorhynchus; Culicoides furens, Culiseta inornata, CuliSeta melanura, Cuterebra spp., Dacus cucurbitae, Dacus oleae, Dasineura brassicae, Dasineura oxycoccana, Della spp. such as D. antique, D. coarctata, D. platura, D. radicum; Dermatobia hominis, Drosophila spp. such as D. suzukii, Fannia spp. such as F. canicularis; Gastraphilus spp. such as G. intestinalis; Geomyza tipunctata, Glossina spp. such as G. fuscipes, G. morsitans, G. palpalis, G. tachinoides, Haematobia irritans, Haplodiplosis equestris, Hippelates spp., Hylemyia spp. such as H. platura; Hypoderma spp. such as H. lineata; Hyppobosca spp., Hydrellia philippina, Leptoconops torrens, Liriomyza spp. such as L. sativae, L. trifolii Lucilia spp. such as L. caprin, L. cuprina, L. sericata; Lycoria pectoralis, Mansonia titillanus, Mayetiola spp. such as M. destructor, Musca spp. such as M. autumnalis, M. domestica; Muscina stabulans, Oestrus spp. such as O. ovis; Opomyza florum, Oscinella spp. such as O. frit; Orseolia oryzae, Pegomya hysocyami, Phlebotomus argentipes, Phorbia spp. such as P. antiqua, P. brassicae, P. coarctata, Phytomyza gymnostoma, Prosimulium mixturn, Psila rosae, Psorophora columbiae, Psorophora discolor, Rhagoletis spp. such as R. cerasi, R. cingulate, R. indifferens, R. mendax, R. pomonella; Rivellia quadrifasciata, Sarcophaga spp. such as S. haemorrhoidalis; Simulium vittatum, Sitodiplosis mosellana, Stomoxys spp. such as S. calcitrans; Tabanus spp. such as T. atratus, T. bovinus, T. lineola, T. Tannia spp., Thecodiplosis japonensis, Tipula oleracea, Tipula paludosa, and Wohlfahrtia spp;

insects from the order of Thysanoptera for example, Baliothrips biformis, Dichromothrips corbetti, Dichromothrips ssp., Echinothrips americanus, Enneothrips flavens, Frankliniella spp. such as F. fusca, F. occidentalis, F. tritici; Heliothrips spp., Hercinothrips femoralis, Kakothrips spp., Microcephalothrips abdominalis, Neohydatothrips samayunkur, Pezothrips kellyanus, Rhipiphorothrips cruentatus, Scirtothrips spp. such as S. citri, S. dorsalis, S. perseae; Stenchaetothrips spp., Taeniothrips cardamoni, Taeniothrips inconsequens, Thrips spp. such as T. imagines, T. hawaiiensis, T. oryzae, T. palmi, T. parvispinus, T. tabaci;

insects from the order of Hemiptera for example, Acizzia jamatonica, Acrosternum spp. such as A. hilare; Acyrthosipon spp. such as A. onobrychis, A. pisum; Adelges laricis, Adelges tsugae, Adelphocoris spp., such as A. rapidus, A. superbus; Aeneolamia spp., Agonoscena spp., Aulacorthum solani, Aleurocanthus woglumi, Aleurodes spp., Aleurodicus disperses, Aleurolobus barodensis, Aleurothrixusspp., Amrasca spp., Anasa tristis, Antestiopsis spp., Anuraphis cardui, Aonidiella spp., Aphanostigma piri, Aphidula nasturtii, Aphis spp. such as A. craccivora, A. fabae, A. forbesi A. gossypii A. grossulariae, A. maidiradicis, A. pomi, A. sambuci, A. schneideri, A. spiraecola; Arboridia Apicalus; Arilus critatus, Aspidiella spp., Aspidiotus spp., Atanus spp., Aulacaspis yasumatsui, Aulacorthum solani, Bactericera cockerelli (Paratrioza cockerelli), Bemisia spp. such as B. argentifolii, B. tabaci (Aleurodes tabaci); Blissus spp. such as B. leucopterus; Brachycaudus spp. such as B. cardui, B. helichrysi, B. persicae, B. prunicola; Brachycolus spp., Brachycorynella asparagi, Brevicoryne brassicae, Cacopsylla spp. such as C. fulguralis, C. pyricola (Psylla piri); Calligypona marginata, Caloconis spp., Campylomma livida Capitophorus horni, Cameocephala fulgida, Cavelerius spp., Ceraplastes spp., Ceratovacuna lanigera, Ceroplastes ceriferus, Cerosipha gossypii, Chaetosiphon fragaefolii Chionaspis tegalensis, Chlorita onukii, Chromaphis juglandicola, Chlysomphalus ficus, Cicadulina mbila, Cimex spp. such as C. hemipterus, C. lectularius; Coccomytilus halli, Coccus spp. such as C. hesperidum, C. pseudomagnoliarum; Corythucha arcuata, Creontiades dilutus, Cryptomyzus ribis, Chrysomphalus aonidum, Cryptomyzus ribis, Ctenarytaina spatulata, Cyrtopeltis notatus, Dalbulus spp., Dasynus piperis, Dialeurodes spp. such as D. citrifolii; Dalbulus maidis, Diaphorina spp. such as D. citri, Diaspis spp. such as D. bromeliae; Dichelops furcatus, Diconocoris hewetti, Doralis spp., Dreyfusia nordmannianae, Dreyfusia piceae, Drosicha spp., Dysaphis spp. such as D. plantaginea, D. pyri, D. radicola; Dysaulacorthum pseudosolani, Dysdercus spp. such as D. cingulatus, D. intermedius; Dysmicoccus spp., Edessa spp., Geocoris spp., Empoasca spp. such as E. fabae, E. solana; Epidiaspis leperii, Eriosoma spp. such as E. lanigerum, E. pyricola; Erythroneura spp., Eurygaster spp. such as E. integriceps; Euscelis bilobatus, Euschistus spp. such as E. heros, E. impictiventris, E. servus; Fiorinia theae, Geococcus coffeae, Glycaspis brimblecombei, Halyomorpha spp. such as H. halys; Hellopeltis spp., Homalodisca vitripennis (=H. coagulata), Horcias nobilellus, Hyalopterus pruni, Hyperomyzus lactucae, Icerya spp. such as I. purchase; Idiocerus spp., Idioscopus spp., Laodelphax striatellus, Lecanium spp., Lecanoideus floccissimus, Lepidosaphes spp. such as L. ulmi, Leptocorisa spp., Leptoglossus phyllopus, Lipaphis erysimi, Lygus spp. such as L. hesperus, L. lineolaris, L. pratensis; Maconellicoccus hirsutus, Marchalina hellenica, Macropes excavatus, Macrosiphum spp. such as M. rosae, M. avenae, M. euphorbiae; Macrosteles quadrilineatus, Mahanarva fimbriolata, Megacopta cribraria, Megoura viciae, Melanaphlis pyrarius, Melanaphis sacchari, Melanocallis (=Tinocallis) coyaefoliae, Metcafiella spp., Metopolophium dirhodum, Monellia costalis, Monelliopsis pecanis, Myzocalillis coryli, Murgantia spp., Myzus spp. such as M. ascalonicus, M. cerasi, M. nicotianae, M. persicae, M. varians; Nasonovia ribis nigri, Neotoxoptera formosana, Neomegalotomus spp, Nephotettix spp. such as N. malayanus, N. nigropictus, N. parvus, N. virescens, Nezara spp. such as N. viridula; Nilaparvata lugens, Nysius huttoni, Oebalus spp. such as O. pugnax; Oncometopia spp., Orthezia praelonga, Oxycaraenus hyalinipennis, Parabemisia myricae, Parlatoria spp., Parthenolecanium spp. such as P. corni, P. persicae; Pemphigus spp. such as P. bursarius, P. populivenae; Peregrinus maidis, Perkinsiella saccharicida, Phenacoccus spp. such as P. aceris, P. gossypii; Phloeomyzus passerinii Phorodon humuli, Phylloxera spp. such as P. devastatrix, Piesma quadrata, Piezodorus spp. such as P. guildinii; Pinnaspis aspidistrae, Planococcus spp. such as P. citri, P. ficus; Prosapia bicincta, Protopulvinaria pyriformis, Psallus seriatus, Pseudacysta persea, Pseudaulacaspis pentagona, Pseudococcus spp. such as P. comstocki; Psylla spp. such as P. mali; Pteromalus spp., Pulvinaria amygdali, Pyrilla spp., Quadraspidiotus spp., such as a perniciosus; Quesada gigas, Rastrococcus spp., Reduvius senilis, Rhizoecus americanus, Rhodnius spp., Rhopalomyzus ascalonicus, Rhopalosiphum spp. such as R. pseudobrassicas, R. insertum, R. maidis, R. padi; Sagatodes spp., Sahlbergella singularis, Saissetia spp., Sappaphis mala, Sappaphis mali, Scaptocoris spp., Scaphoides titanus, Schizaphis graminum, Schizoneura lanuginosa, Scotinophora spp., Selenaspidus articulatus, Sitobion avenae, Sogata spp., Sogatella furcifera, Solubea insularis, Spissistilus festinus (=Stictocephala festina), Stephanitis nashi, Stephanitis pyrioides, Stephanitis takeyai, Tenalaphara malayensis, Tetraleurodes perseae, Therioaphis maculate, Thyanta spp. such as T. accerra, T. perditor; Tibraca spp., Tomaspis spp., Toxoptera spp. such as T. aurantii; Trialeurodes spp. such as T. abutilonea, T. ricin, T. vaporariorum; Triatoma spp., Trioza spp., Typhlocyba spp., Unaspis spp. such as U. citri, U. yanonensis; and Viteus vitifolii,

Insects from the order Hymenoptera for example Acanthomyops interjectus, Athalia rosae, Atta spp. such as A. capiguara, A. cephalotes, A. cephalotes, A. laevigata, A. robusta, A. sexdens, A. texana, Bombus spp., Brachymyrmex spp., Camponotus spp. such as C. floridanus, C. pennsylvanicus, C. modoc; Cardiocondyla nuda, Chalibion sp, Crematogaster spp., Dasymutilla occidentalis, Diprion spp., Dolichovespula maculata, Dorymyrmex spp., Dryocosmus kuriphilus, Formica spp., Hoplocampa spp. such as H. minuta, H. testudinea; Iridomyrmex humilis, Lasius spp. such as L. niger, Linepithema humile, Liometopum spp., Leptocybe invasa, Monomorium spp. such as M. pharaonis, Monomorium, Nylandria fulva, Pachycondyla chinensis, Paratrechina longicornis, Paravespula spp., such as P. germanica, P. pennsylvanica, P. vulgaris; Pheidole spp. such as P. megacephala; Pogonomyrmex spp. such as P. barbatus, P. californicus, Polistes rubiginosa, Prenolepis impairs, Pseudomyrmex gracilis, Schelipron spp., Sirex cyaneus, Solenopsis spp. such as S. geminata, S. invicta, S. molesta, S. richteri, S. xyloni, Sphecius speciosus, Sphex spp., Tapinoma spp. such as T. melanocephalum, T. sessile; Tetramorium spp. such as T. caespitum, T. bicarinatum, Vespa spp. such as V. crabro; Vespula spp. such as V. squamosal; Wasmannia auropunctata, Xylocopa sp;

Insects from the order Orthoptera for example Acheta domesticus, Calliptamus italicus, Chortoicetes terminifera, Ceuthophilus spp., Diastrammena asynamora, Dociostaurus maroccanus, Gryllotalpa spp. such as G. africana, G. gryllotalpa; Gryllus spp., Hieroglyphus daganensis, Kraussaria angulifera, Locusta spp. such as L. migratoria, L. pardalina; Melanoplus spp. such as M. bivittatus, M. femurrubrum, M. mexicanus, M. sanguinipes, M. spretus; Nomadacris septemfasciata, Oedaleus senegalensis, Scapteriscus spp., Schistocerca spp. such as S. americana, S. gregaria, Stemopelmatus spp., Tachycines asynamorus, and Zonozerus variegatus;

Pests from the Class Arachnida for example Acari, e.g. of the families Argasidae, Ixodidae and Sarcoptidae, such as Amblyomma spp. (e.g. A. americanum, A. variegatum, A. maculatum), Argas spp. such as A. persicu), Boophilus spp. such as B. annulatus, B. decoloratus, B. microplus, Dermacentor spp. such as D. silvarum, D. andersoni, D. variabilis, Hyalomma spp. such as H. truncatum, Ixodes spp. such as I. ricinus, I. rubicundus, I. scapularis, I. holocyclus, I. pacificus, Rhipicephalus sanguineus, Ornithodorus spp. such as O. moubata, O. hermsi, O. turicata, Ornithonyssus bacoti, Otobius megnini, Dermanyssus gallinae, Psoroptes spp. such as P. ovis, Rhipicephalus spp. such as R. sanguineus, R. appendiculatus, Rhipicephalus evertsi, Rhizoglyphus spp., Sarcoptes spp. such as S. Scabiei; and Family Eriophyidae including Aceria spp. such as A. sheldoni, A. anthocoptes, Acallitus spp., Aculops spp. such as A. lycopersici, A. pelekassi; Aculus spp. such as A. schlechtendali; Colomerus vitis, Epitrimerus pyri, Phyllocoptruta oleivora; Eriophytes ribis and Eriophyes spp. such as Eriophyes sheldoni; Family Tarsonemidae including Hemitarsonemus spp., Phytonemus pallidus and Polyphagotarsonemus latus, Stenotarsonemus spp. Steneotarsonemus spinki; Family Tenuipalpidae including Brevipalpus spp. such as B. phoenicis; Family Tetranychidae including Eotetranychus spp., Eutetranychus spp., Oligonychus spp., Petrobia latens, Tetranychus spp. such as T. cinnabarinus, T. evansi, T. kanzawai, T, pacificus, T. phaseulus, T. telarius and T. urticae; Bryobia praetiosa; Panonychus spp. such as P. ulmi, P. citri; Metatetranychus spp. and Oligonychus spp. such as O. pratensis, O. perseae, Vasates lycopersici; Raoiella indica, Family Carpoglyphidae including Carpoglyphus spp.; Penthaleidae spp. such as Halotydeus destructor, Family Demodicidae with species such as Demodex spp.; Family Trombicidea including Trombicula spp.; Family Macronyssidae including Ornothonyssus spp.; Family Pyemotidae including Pyemotes tritici; Tyrophagus putrescentiae; Family Acaridae including Acarus siro; Family Araneida including Latrodectus mactans, Tegenaria agrestis, Chiracanthium sp, Lycosa sp Achaearanea tepidariorum and Loxosceles reclusa;

Pests from the Phylum Nematoda, for example, plant parasitic nematodes such as root-knot nematodes, Meloidogyne spp. such as M. hapla, M. incognita, M. javanica; cyst-forming nematodes, Globodera spp. such as G. rostochiensis; Heterodera spp. such as H. avenae, H. glycines, H. schachtii, H. trifolii; Seed gall nematodes, Anguina spp.; Stem and foliar nematodes, Aphelenchoides spp. such as A. besseyi; Sting nematodes, Belonolaimus spp. such as B. Iongicaudatus; Pine nematodes, Bursaphelenchus spp. such as B. lignicolus, B. xylophilus; Ring nematodes, Criconema spp., Criconemella spp. such as C. xenoplax and C. ornata; and, Criconemoides spp. such as Criconemoides informis; Mesocriconema spp.; Stem and bulb nematodes, Ditylenchus spp. such as D. destructor, D. dipsaci; Awl nematodes, Dolichodorus spp.; Spiral nematodes, Heliocotylenchus multicinctus; Sheath and sheathoid nematodes, Hemicycliophora spp. and Hemicriconemoides spp.; Hirshmanniella spp.; Lance nematodes, Hoploaimus spp.; False rootknot nematodes, Nacobbus spp.; Needle nematodes, Longidorus spp. such as L. elongatus; Lesion nematodes, Pratylenchus spp. such as P. brachyurus, P. neglectus, P. penetrans, P. curvitatus, P. goodeyi; Burrowing nematodes, Radopholus spp. such as R. similis; Rhadopholus spp.; Rhodopholus spp.;Reniform nematodes, Rotylenchus spp. such as R. robustus, R. reniformis; Scutellonema spp.; Stubby-root nematode, Trichodorus spp. such as T. obtusus, T. primitivus; Paratrichodorus spp. such as P. minor; Stunt nematodes, Tylenchorhynchus spp. such as T. claytoni, T. dubius, Citrus nematodes, Tylenchulus spp. such as T. semipenetrans; Dagger nematodes, Xiphinema spp.; and other plant parasitic nematode species;

Insects from the order Isoptera for example Calotermes flavicollis, Coptotermes spp. such as C. formosanus, C. gestroi, C. acinaciformis; Cornitermes cumulans, Cryptotermes spp. such as C. brevis, C. cavifrons; Globitermes sulfureus, Heterotermes spp. such as H. aureus, H. longiceps, H. tenuis; Leucotermes flavipes, Odontotermes spp., Incisitermes spp. such as I. minor, I. Snyder, Marginitermes hubbardi, Mastotermes spp. such as M. darwiniensis Neocapritermes spp. such as N. opacus, N. parvus; Neotermes spp., Procornitermes spp., Zootermopsis spp. such as Z. angusticollis, Z. nevadensis, Reticulitermes spp. such as R. hesperus, R. tibialis, R. speratus, R. flavipes, R. grassei, R. lucifugus, R. santonensis, R. virginicus; Termes natalensis,

Insects from the order Blattaria for example Blatta spp. such as B. orientalis, B. lateralis; Blattella spp. such as B. asahinae, B. germanica; Leucophaea maderae, Panchlora nivea, Periplaneta spp. such as P. americana, P. australasiae, P. brunnea, P. fuligginosa, P. japonica, Supella longipalpa, Parcoblatta pennsylvanica, Eurycotis floridana, Pycnoscelus surinamensis,

Insects from the order Siphonoptera for example Cediopsylla simples, Ceratophyllus spp., Ctenocephalides spp. such as C. felis, C. canis, Xenopsylla cheopis, Pulex irritans, Trichodectes canis, Tunga penetrans, and Nosopsyllus fasciatus, Insects from the order Thysanura for example Lepisma saccharina, Ctenolepisma urbana, and Thermobia domestica, Pests from the class Chilopoda for example Geophilus spp., Scutigera spp. such as Scutigera coleoptrata; Pests from the class Diplopoda for example Blaniulus guttulatus, Julus spp., Narceus spp., Pests from the class Symphyla for example Scutigerella immaculata, Insects from the order Dermaptera, for example Forficula auricularia, Insects from the order Collembola, for example Onychiurus spp., such as Onychiurus armatus, Pests from the order Isopoda for example, Armadillidium vulgare, Oniscus asellus, Porcellio scaber, Insects from the order Phthiraptera, for example Damalinia spp., Pediculus spp. such as Pediculus humanus capitis, Pediculus humanus corporis, Pediculus humanus humanus, Pthirus pubis, Haematopinus spp. such as Haematopinus eurysternus, Haematopinus suis, Linognathus spp. such as Linognathus Bovicola bovis, Menopon gallinae, Menacanthus stramineus and Solenopotes capillatus, Trichodectes spp., Examples of further pest species which may be controlled by compounds of fomula (I) include: from the Phylum Mollusca, class Bivalvia, for example, Dreissena spp.; class Gastropoda, for example, Arion spp., Biomphalaria spp., Bulinus spp., Deroceras spp., Galba spp., Lymnaea spp., Oncomelania spp., Pomacea canaliclata, Succinea spp.; from the class of the helminths, for example, Ancylostoma duodenale, Ancylostoma ceylanicum, Acylostoma braziliensis, Ancylostoma spp., Ascaris lubricoides, Ascaris spp., Brugia malayi, Brugia timori, Bunostomum spp., Chabertia spp., Clonorchis spp., Cooperia spp., Dicrocoelium spp., Dictyocaulus filaria, Diphyllobothrium latum, Dracunculus medinensis, Echinococcus granulosus, Echinococcus multilocularis, Enterobius vermicularis, Faciola spp., Haemonchus spp. such as Haemonchus contortus; Heterakis spp., Hymenolepis nana, Hyostrongulus spp., Loa Loa, Nematodirus spp., Oesophagostomum spp., Opisthorchis spp., Onchocerca volvulus, Ostertagia spp., Paragonimus spp., Schistosomen spp., Strongyloides fuelleborni, Strongyloides stercora lis, Stronyloides spp., Taenia saginata, Taenia solium, Trichinella spiralis, Trichinella nativa, Trichinella britovi, Trichinella nelsoni, Trichinella pseudopsiralis, Trichostrongulus spp., Trichuris trichuria, Wuchereria bancrofti.

The present invention also relates to a mixture of at least one compound of the present invention with at least one mixing partner as defined herein after. Preferred are binary mixtures of one compound of the present invention as component I with one mixing partner as defined herein after as component II. Preferred weight ratios for such binary mixtures are from 5000:1 to 1:5000, preferably from 1000:1 to 1:1000, more preferably from 100:1 to 1:100, particularly preferably from 10:1 to 1:10. In such binary mixtures, components I and II may be used in equal amounts, or an excess of component I, or an excess of component II may be used.

Mixing partners can be selected from pesticides, in particular insecticides, nematicides, and acaricides, fungicides, herbicides, plant growth regulators, fertilizers, and the like. Preferred mixing partners are insecticides, nematicides and fungicides.

The following list M of pesticides, grouped and numbered according the Mode of Action Classification of the Insecticide Resistance Action Committee (IRAC), together with which the compounds of the present invention can be used and with which potential synergistic effects might be produced, is intended to illustrate the possible combinations, but not to impose any limitation: M.1 Acetylcholine esterase (AChE) inhibitors from the class of: M.1A carbamates, for example aldicarb, alanycarb, bendiocarb, benfuracarb, butocarboxim, butoxycarboxim, carbaryl, carbofuran, carbosulfan, ethiofencarb, fenobucarb, formetanate, furathiocarb, isoprocarb, methiocarb, methomyl, metolcarb, oxamyl, pirimicarb, propoxur, thiodicarb, thiofanox, trimethacarb, XMC, xylylcarb and triazamate; or from the class of M.1B organophosphates, for example acephate, azamethiphos, azinphos-ethyl, azinphosmethyl, cadusafos, chlorethoxyfos, chlorfenvinphos, chlormephos, chlorpyrifos, chlorpyrifos-methyl, coumaphos, cyanophos, demeton-S-methyl, diazinon, dichlorvos/DDVP, dicrotophos, dimethoate, dimethylvinphos, disulfoton, EPN, ethion, ethoprophos, famphur, fenamiphos, fenitrothion, fenthion, fosthiazate, heptenophos, imicyafos, isofenphos, isopropyl O- (methoxyaminothio-phosphoryl) salicylate, isoxathion, malathion, mecarbam, methamidophos, methidathion, mevinphos, monocrotophos, naled, omethoate, oxydemeton-methyl, parathion, parathion-methyl, phenthoate, phorate, phosalone, phosmet, phosphamidon, phoxim, pirimiphos-methyl, profenofos, propetamphos, prothiofos, pyraclofos, pyridaphenthion, quinalphos, sulfotep, tebupirimfos, temephos, terbufos, tetrachlorvinphos, thiometon, triazophos, trichlorfon and vamidothion;

M.2. GABA-gated chloride channel antagonists such as: M.2A cyclodiene organochlorine compounds, as for example endosulfan or chlordane; or M.2B fiproles (phenylpyrazoles), as for example ethiprole, fipronil, flufiprole, pyrafluprole and pyriprole;

M.3 Sodium channel modulators from the class of M.3A pyrethroids, for example acrinathrin, allethrin, d-cis-trans allethrin, d-trans allethrin, bifenthrin, bioallethrin, bioallethrin S-cylclopentenyl, bioresmethrin, cycloprothrin, cyfluthrin, beta-cyfluthrin, cyhalothrin, lambda-cyhalothrin, gamma-cyhalothrin, cypermethrin, alpha-cypermethrin, beta-cypermethrin, theta-cypermethrin, zeta-cypermethrin, cyphenothrin, deltamethrin, empenthrin, esfenvalerate, etofenprox, fenpropathrin, fenvalerate, flucythrinate, flumethrin, tau-fluvalinate, halfenprox, heptafluthrin, imiprothrin, meperfluthrin,metofluthrin, momfluorothrin, permethrin, phenothrin, prallethrin, profluthrin, pyrethrin (pyrethrum), resmethrin, silafluofen, tefluthrin, tetramethylfluthrin, tetramethrin, tralomethrin and transfluthrin; or M.3B sodium channel modulators such as DDT or methoxychlor;

M.4 Nicotinic acetylcholine receptor agonists (nAChR) from the class of M.4A neonicotinoids: acetamiprid, clothianidin, cycloxaprid, dinotefuran, imidacloprid, nitenpyram, thiacloprid and thiamethoxam; or the compounds M.4A.2: (2E+1-[(6-Chloropyridin-3-y)methyl]-M-nitro-2pentylidenehydrazinecarboximidamide; or M4.A.3: 1-[(6-Chloropyridin-3-yl) methyl]-7-methyl-8nitro-5-propoxy-1,2,3,5,6,7-hexahydroimidazo[1,2-a]pyridine; or from the class M.4B nicotine;

M.5 Nicotinic acetylcholine receptor allosteric activators from the class of spinosyns: spinosad or spinetoram;

M.6 Chloride channel activators from the class of avermectins and milbemycins, for example abamectin, emamectin benzoate, ivermectin, lepimectin or milbemectin;

M.7 Juvenile hormone mimics: M.7A juvenile hormone analogues as hydroprene, kinoprene and methoprene; or others as M.7B fenoxycarb or M.7C pyriproxyfen;

M.8 miscellaneous non-specific (multi-site) inhibitors, for example M.8A alkyl halides as methyl bromide and other alkyl halides, or M.8B chloropicrin, or M.8C sulfuryl fluoride, or M.8D borax, or M.8E tartar emetic;

M.9 Selective homopteran feeding blockers: M.9B pymetrozine, or M.9C flonicamid;

M.10 Mite growth inhibitors: M.10A clofentezine, hexythiazox and diflovidazin, or M.10B etoxazole;

M.11 Microbial disruptors of insect midgut membranes, for example bacillus thuringiensis or bacillus sphaericus, and the insecticdal proteins they produce such as bacillus thuringiensis subsp. israelensis, bacillus sphaericus, bacillus thuringiensis subsp. aizawai, bacillus thuringiensis subsp. kurstaki and bacillus thuringiensis subsp. tenebrionis, or the Bt crop proteins: Cry1Ab, Cry1Ac, Cry1Fa, Cry2Ab, mCry3A, Cry3Ab, Cry3Bb and Cry34/35Ab1;

M.12 Inhibitors of mitochondrial ATP synthase: M.12A diafenthiuron, or M.12B organotin miticides such as azocyclotin, cyhexatin or fenbutatin oxide, or M.12C propargite, or M.12D tetradifon;

M.13 Uncouplers of oxidative phosphorylation via disruption of the proton gradient: chlorfenapyr, DNOC or sulfluramid;

M.14 Nicotinic acetylcholine receptor (nAChR) channel blockers, for example nereistoxin analogues as bensultap, cartap hydrochloride, thiocyclam or thiosultap sodium;

M.15 Inhibitors of the chitin biosynthesis type 0, such as benzoylureas as for example bistrifluron, chlorfluazuron, diflubenzuron, flucycloxuron, flufenoxuron, hexaflumuron, lufenuron, novaluron, noviflumuron, teflubenzuron or triflumuron;

M.16 Inhibitors of the chitin biosynthesis type 1, as for example buprofezin;

M.17 Moulting disruptors, Dipteran, as for example cyromazine;

M.18 Ecdyson receptor agonists such as diacylhydrazines, for example methoxyfenozide, tebufenozide, halofenozide, fufenozide or chromafenozide;

M.19 Octopamin receptor agonists, as for example amitraz;

M.20 Mitochondrial complex III electron transport inhibitors, for example M.20A hydramethylnon, or M.20B acequinocyl, or M.20C fluacrypyrim;

M.21 Mitochondrial complex I electron transport inhibitors, for example M.21A METI acaricides and insecticides such as fenazaquin, fenpyroximate, pyrimidifen, pyridaben, tebufenpyrad or tolfenpyrad, or M.21B rotenone;

M.22 Voltage-dependent sodium channel blockers: M.22A indoxacarb, or M.22B metaflumizone, or M.22B.1: 2-[2-(4-Cyanophenyl)-1-[3-(trifluoromethyl)phenyl]ethylidene[-N-[(4-(difluoromethoxy)phenyl]-hydrazinecarboxamide or M.22B.2: N-(3-Chloro-2-methylphenyl)-2-[(4chlorophenyl)[4-[methyl(methylsulfonyl)amino]phenyl]methylene]hydrazinecarboxamide;

M.23 Inhibitors of the of acetyl CoA carboxylase, such as Tetronic and Tetramic acid derivatives, for example spirodiclofen, spiromesifen or spirotetramat;

M.24 Mitochondrial complex IV electron transport inhibitors, for example M.24A phosphine such as aluminium phosphide, calcium phosphide, phosphine or zinc phosphide, or M.24B cyanide;

M.25 Mitochondrial complex II electron transport inhibitors, such as beta-ketonitrile derivatives, for example cyenopyrafen or cyflumetofen;

M.28 Ryanodine receptor-modulators from the class of diamides: flubendiamide, chlorantraniliprole (rynaxypyr®), cyantraniliprole (cyazypyr®), tetraniliprole, or the phthalamide compounds

M.28.1: (R)-3-Chloro-N1-{2-methyl-4-[1,2,2,2-tetrafluoro-1-(trifluormethyl)ethyl]phenyl}-N2-(1methyl-2-methylsulfonylethyl)phthalamid and M.28.2: (S)-3-Chlor-N1-{2-methyl-4-[1,2,2,2-tetrafluoro-1-(trifluoromethyl)ethyl]phenyl}-N2-(1-methyl-2-methylsulfonylethyl)phthalamid, or the compound M.28.3: 3-bromo-N-{2-bromo-4-chloro-6-[(1-cyclopropylethyl)carbamoyl]phenyl}-1-(3chloropyridin-2-yl)-1H-pyrazole-5-carboxamide (cyclaniliprole), or the compound M.28.4: methyl2-[3,5-dibromo-2-({[3-bromo-1-(3-chloropyridin-2-yl)-1H-pyrazol-5-yl]carbonyl}amino)benzoyl]-1,2-dimethylhydrazinecarboxylate; or a compound selected from M.28.5a) to M.28.5d) and M.28.5h) to M.28.5I): M.28.5a) N-[4,6-dichloro-2-[(diethyl-lambda-4-sulfanylidene)carbamoyl]-phenyl]-2-(3-chloro-2-pyridyl)-5-(trifluoromethyl)pyrazole-3-carboxamide; M.28.5b) N-[4-chloro-2-[(diethyl-lambda-4-sulfanylidene)carbamoyl]-6-methyl-phenyl]-2-(3-chloro-2-pyridyl)-5-(trifluoromethyl)pyrazole-3-carboxamide; M.28.5c) N-[4-chloro-2-[(di-2-propyl-lambda-4-sulfanylidene)carbamoyl]-6-methyl-phenyl]-2-(3-chloro-2-pyridyl)-5-(trifluoromethyl)pyrazole-3-carboxamide; M.28.5d) N-[4,6-dichloro-2-[(di-2-propyl-lambda-4-sulfanylidene)carbamoyl]-phenyl]-2-(3-chloro-2-pyridyl)-5-(trifluoromethyl)pyrazole-3-carboxamide; M.28.5h) N-[4,6-dibromo-2-[(diethyl-lambda-4-sulfanylidene)carbamoyl]-phenyl]-2-(3-chloro-2-pyridyl)-5-(trifluoromethyl)pyrazole-3-carboxamide; M.28.5i) N-[2-(5-Amino-1,3,4-thiadiazol-2-yl)-4-chloro-6-methylphenyl]-3-bromo-1-(3-chloro-2-pyridinyl)-1H-pyrazole-5-carboxamide; M.28.5j) 3-Chloro-1-(3-chloro-2-pyridinyl)-N-[2,4-dichloro-6-[[(1-cyano-1-methylethyl)amino]carbonyl]phenyl]-1H-pyrazole-5-carboxamide; M.28.5k) 3-Bromo-N-[2,4-dichloro-6-(methylcarbamoyl)phenyl]-1-(3,5-dichloro-2-pyridyl)-1H-pyrazole-5-carboxamide; M.28.51) N-[4-Chloro-2-[[(1,1-dimethylethyl)amino]carbonyl]-6-methylphenyl]-1-(3-chloro-2-pyridinyl)-3-(fluoromethoxy)-1H-pyrazole-5-carboxamide; or

M.28.6: cyhalodiamide; or;

M.29. insecticidal active compounds of unknown or uncertain mode of action, as for example afidopyropen, afoxolaner, azadirachtin, amidoflumet, benzoximate, bifenazate, broflanilide, bromopropylate, chinomethionat, cryolite, dicloromezotiaz, dicofol, flufenerim, flometoquin, fluensulfone, fluhexafon, fluopyram, flupyradifurone, fluralaner, metoxadiazone, piperonyl butoxide, pyflubumide, pyridalyl, pyrifluquinazon, sulfoxaflor, tioxazafen, triflumezopyrim, or M.29.3: 11-(4-chloro-2,6-dimethylphenyl)-12-hydroxy-1,4-dioxa-9-azadispiro[4.2.4.2]-tetradec-11-en-10-one, or M.29.4: 3-(4′-fluoro-2,4-dimethylbiphenyl-3-yl)-4-hydroxy-8-oxa-1-azaspiro[4.5]dec-3-en-2-one, or M.29.5: 1-[2-fluoro-4-methyl-5-[(2,2,2-trifluoroethyl)sulfinyl]phenyl]-3-(trifluoromethyl)-1H-1,2,4-triazole-5-amine, or actives on basis of bacillus firmus (Votivo, 1-1582); or a compound selected from M.29.6, wherein the compound is selected from M.29.6a) to M.29.6k): M.29.6a) (E/Z)-N-[1-[(6-chloro-3-pyridyl)methyl]-2-pyridylidene]-2,2,2-trifluoro-acetamide; M.29.6b) (E/Z)-N-[1-[(6-chloro-5-fluoro-3-pyridyl)methyl]-2-pyridylidene]-2,2,2-trifluoro-acetamide; M.29.6c) (E/Z)-2,2,2-trifluoro-N-[1-[(6-fluoro-3-pyridyl)methyl]-2-pyridylidene]acetamide; M.29.6d) (E/Z)-N-[1-[(6-bromo-3-pyridyl)methyl]-2-pyridylidene]-2,2,2-trifluoro-acetamide; M.29.6e) (E/Z)-N-[1-[1-(6-chloro-3-pyridyl)ethyl]-2-pyridylidene]-2,2,2-trifluoro-acetamide; M.29.6f) (E/Z)-N-[1-[(6-chloro-3-pyridyl)methyl]-2-pyridylidene]-2,2-difluoro-acetamide; M.29.6g) (E/Z)-2-chloro-N-[1-[(6-chloro-3-pyridyl)methyl]-2-pyridylidene]-2,2-difluoro-acetamide; M.29.6h) (E/Z)-N-[1-[(2-chloropyrimidin-5-yl)methyl]-2-pyridylidene]-2,2,2-trifluoro-acetamide; M.29.6i) (E/Z)-N-[1-[(6-chloro-3-pyridyl)methyl]-2-pyridylidene]-2,2,3,3,3-pentafluoro-propanamide.); M.29.6j) N-[1-[(6-chloro-3-pyridyl)methyl]-2-pyridylidene]-2,2,2-trifluoro-thioacetamide; or M.29.6k) N-[1-[(6-chloro-3-pyridyl)methyl]-2-pyridylidene]-2,2,2-trifluoro-N′-isopropyl-acetamidine; or M.29.8: fluazaindolizine; or M.29.9.a): 4-[5-(3,5-dichlorophenyl)-5-(trifluoromethyl)-4H-isoxazol-3-yl]-2-methyl-N-(1-oxothietan-3- yl)benzamide; or M.29.9.b): fluxametamide; or M.29.10: 5-[3-[2,6-dichloro-4-(3,3-dichloroallyloxy)phenoxy]propoxy]-1H-pyrazole; or a compound selected from M.29.11, wherein the compound is selected from M.29.11b) to M.29.11p): M.29.11.b) 3-(benzoylmethylamino)-N-[2-bromo-4-[1 ,2,2,3,3,3-hexafluoro-1-(trifluoromethyl)propyl]-6-(trifluoromethyl)phenyl]-2-fluoro-benzamide; M.29.11.c) 3-(benzoylmethylamino)-2-fluoro-N-[2-iodo-4-[1 ,2,2,2-tetrafluoro-1-(trifluoromethyl)ethyl]-6-(trifluoromethyl)phenyl]-benzamide; M.29.11.d) N-[3-[[[2-iodo-4-[1,2,2,2-tetrafluoro-1-(trifluoromethyl)ethyl]-6-(trifluoromethyl)phenyl]amino]carbonyl]phenylFN-methyl- benzamide; M.29.11.e) N-[3-[[[2-bromo-4-[1 ,2,2,2-tetrafluoro-1-(trifluoromethyl)ethyl]-6-(trifluoromethyl)phenyl]amino]carbonyl]-2-fluorophenyl]-4-fluoro-N-methyl-benzamide; M.29.11.f) 4-fluoro-N-[2-fluoro-3-[[[2-iodo-4-[1,2,2,2-tetrafluoro-1-(trifluoromethyl)ethyl]-6-(trifluoromethyl)phenyl]amino]carbonyl]phenyl]N-methyl-benzamide; M.29.11.g) 3-fluoro-N-[2-fluoro-3-[[[2-iodo-4-[1,2,2,2-tetrafluoro-1-(trifluoromethyl)-ethyl]-6-(trifluoromethyl)phenyl]amino]carbonyl]phenyl]-N-methyl-benzamide; M.29.11.h) 2-chloro-N-[3-[[[2-iodo-4-[1,2,2,2-tetrafluoro-1- (trifluoromethyl)-ethyl]-6-(trifluoromethyl)phenyl]amino]carbonyl]phenyl]-3-pyridinecarboxamide; M.29.11.i) 4-cyano-N-[2-cyano-5-[[2,6-dibromo-4-[1,2,2,3,3,3-hexafluoro-1-(trifluoromethyl)-propyl]phenyl]carbamoyl]phenyl]-2-methyl-benzamide; M.29.11.j) 4-cyano-3-[(4-cyano-2-methyl-benzoyl)amino]-N-[2,6-dichloro-4-[1,2,2,3,3,3-hexafluoro-1-(trifluoromethyl)propyl]phenyl]-2-fluoro-benzamide; M.29.11.k) N-[5-[[2-chloro-6-cyano-4-[1,2,2,3,3,3- hexafluoro-1-(trifluoromethyl)propyl]phenyl]carbamoyl]-2-cyano-phenyl]4-cyano-2-methyl-benzamide; M.29.11.l) N-[5-[[2-bromo-6-chloro-4-[2,2,2-trifluoro-1-hydroxy-1-(trifluoromethyl)ethyl]phenyl]carbamoyl]-2-cyano-phenyl]-4-cyano-2-methyl-benzamide; M.29.11.m) N-[5-[[2-bromo-6-chloro-4-[1,2,2,3,3,3-hexafluoro-1-(trifluoromethyl)-propyl]phenyl]carbamoyl]-2-cyano-phenyl]-4-cyano-2-methyl-benzamide; M.29.11.n) 4-cyano-N-[2-cyano-5-[[2,6-dichloro-4-[1,2,2,3,3,3-hexafluoro-1-(trifluoromethyl)-propyl]phenyl]carbamoyl]phenyl]-2-methyl-benzamide; M.29.11.o) 4-cyano-N-[2-cyano-5-[[2,6-dichloro-4-[1,2,2,2-tetrafluoro-1-(trifluoromethyl)ethyl]phenyl]carbamoyl]phenyl]-2-methyl-benzamide; M.29.11.p) N-[5-[[2-bromo-6-chloro-4-[1,2,2,2-tetrafluoro-1-(trifluoromethyl)ethyl]phenyl]carbamoyl]-2-cyano-phenyl]-4-cyano-2-methyl-benzamide; or

M.29.12, selected from M.29.12a) to M.29.12m): M.29.12.a) 2-(1,3-Dioxan-2-yl)-6-[2-(3-pyridinyl)-5-thiazolyl]-pyridine; M.29.12.b) 2-[6-[2-(5-Fluoro-3-pyridinyl)-5-thiazolyl]-2-pyridinyl]-pyrimidine; M.29.12.c) 2-[6-[2-(3-Pyridinyl)-5-thiazolyl]-2-pyridinyl]-pyrimidine; M.29.12.d) N-Methylsulfonyl-6-[2-(3-pyridyl)thiazol-5-yl]pyridine-2-carboxamide; M.29.12.e) N-Methylsulfonyl-6-[2-(3-pyridyl)thiazol-5-yl]pyridine-2-carboxamide; M.29.12.f) N-Ethyl-N-[4-methyl-2-(3-pyridyl)thiazol-5-yl]-3-methylthio-propanamide; M.29.12.g) N-Methyl-N-[4-methyl-2-(3-pyridyl)thiazol-5-yl]-3-methylthio-propanamide; M.29.12.h) N,2-Dimethyl-N-[4-methyl-2-(3-pyridyl)thiazol-5-yl]-3-methylthio-propanamide; M.29.12.i) N-Ethyl-2-methyl-N-[4-methyl-2-(3-pyridyl)thiazol-5-yl]-3-methylthio-propanamide; M.29.12.j) N-[4-Chloro-2-(3-pyridyl)thiazol-5-yl]-N-ethyl-2-methyl-3-methylthio-propanamide; M.29.12.k) N-[4-Chloro-2-(3-pyridyl)thiazol-5-yl]-N,2-dimethyl-3-methylthio-propanamide; M.29.12.l) N-[4-Chloro-2-(3-pyridyl)thiazol-5-yl]-N-methyl-3-methylthio-propanamide; M.29.12.m) N-[4-Chloro-2-(3-pyridyl)thiazol-5-yl]-N-ethyl-3-methylthio-propanamide; or

M.29.14a) 1-[(6-Chloro-3-pyridinyl)methyl]-1,2,3,5,6,7-hexahydro-5-methoxy-7-methyl-8-nitro-imidazo[1,2-a]pyridine; or M.29.14b) 1-[(6-Chloropyridin-3-yl)methyl]-7-methyl-8-nitro-1,2,3,5,6,7-hexahydroimidazo[1,2-a]pyridin-5-ol; or

M.29.16a) 1-isopropyl-N,5-dimethyl-N-pyridazin-4-yl-pyrazole-4-carboxamide; or M.29.16b) 1-(1,2-dimethylpropyl)-N-ethyl-5-methyl-N-pyridazin-4-yl-pyrazole-4-carboxamide; M.29.16c) N,5-dimethyl-N-pyridazin-4-yl-1-(2,2,2-trifluoro-1-methyl-ethyl)pyrazole-4-carboxamide; M.29.16d) 1-[1-(1-cyanocyclopropypethyl)-ethyl]-N-ethyl-5-methyl-N-pyridazin-4-yl-pyrazole-4-carboxamide; M.29.16e) N-ethyl-1-(2-fluoro-1-methyl-propyl)-5-methyl-N-pyridazin-4-yl-pyrazole-4-carboxamide; M.29.16f) 1-(1,2-dimethylpropyl)-N,5-dimethyl-N-pyridazin-4-yl-pyrazole-4-carboxamide; M.29.16g) 1-[1-(1-cyanocyclopropypethyl)ethyl]-N,5-dimethyl-N-pyridazin-4-yl-pyrazole-4-carboxamide; M.29.16h) N-methyl-1-(2-fluoro-1-methyl-propyl]-5-methyl-N-pyridazin-4-yl-pyrazole-4-carboxamide; M.29.16i) 1-(4,4-difluorocyclohexyl)-N-ethyl-5-methyl-N-pyridazin-4-yl-pyrazole-4-carboxamide; or M.29.16j) 1-(4,4-difluorocyclohexyl)-N,5-dimethyl-N-pyridazin-4-yl-pyrazole-4-carboxamide, or

M.29.17: M.29.17a) to M.29.17j): M.29.17a) N-(1-methylethyl)-2-(3-pyridinyl)-2H-indazole-4-carboxamide; M.29.17b) N-cyclopropyl-2-(3-pyridinyl)-2H-indazole-4-carboxamide; M.29.17c) N-cyclohexyl-2-(3-pyridinyl)-2H-indazole-4-carboxamide; M.29.17d) 2-(3-pyridinyl)-N-(2,2,2-trifluoroethyl)-2H-indazole-4-carboxamide; M.29.17e) 2-(3-pyridinyl)-N-[(tetrahydro-2-furanyl)methyl]-2 H-indazole-5-carboxamide; M.29.17f) methyl 2-[[2-(3-pyridinyl)-2H-indazol-5-yl]carbonyl]hydrazinecarboxylate; M.29.17g) N-[(2,2-difluorocyclopropyl)methyl]-2-(3-pyridinyl)-2H-indazole-5-carboxamide; M.29.17h) N-(2,2-difluoropropyl)-2-(3-pyridinyl)-2H-indazole-5-carboxamide; M.29.17i) 2-(3-pyridinyl)-N-(2-pyrimidinylmethyl)-2H-indazole-5-carboxamide; M.29.17j) N-[(5-methyl-2-pyrazinyl)methyl]-2-(3-pyridinyl)-2H-indazole-5-carboxamide, or

M.29.18: M.29.18a) to M.29.18d): M.29.18a) N-[3-chloro-1-(3-pyridyl)pyrazol-4-yl]-N-ethyl-3-(3,3,3-trifluoropropylsulfanyl)propanamide; M.29.18b) N-[3-chloro-1-(3-pyridyl)pyrazol-4-yl]-N-ethyl-3-(3,3,3-trifluoropropylsulfinyl)propanamide; M.29.18c) N-[3-chloro-1-(3-pyridyl)pyrazol-4-yl-3-[(2,2-difluorocyclopropyl)methylsulfanyl]-N-ethyl-propanamide; M.29.18d) N-[3-chloro-1-(3-pyridyl)pyrazol-4-yl]-3-[(2,2- difluorocyclopropyl)methylsulfinyl]-N-ethyl-propanamide; or M.29.19 sarolaner, or M.29.20 lotilaner.

The commercially available compounds of the group M listed above may be found in The Pesticide Manual, 16th Edition, C. MacBean, British Crop Protection Council (2013) among other publications. The online Pesticide Manual is updated regularly and is accessible through http://bcpcdata.com/pesticide-manual.html.

Another online data base for pesticides providing the ISO common names is http://www.alanwood.net/pesticides.

The M.4 cycloxaprid is known from WO10/069266 and WO 11/069456, M.4A.2, named as guadipyr, is known from WO13/003977, and M.4A.3 (approved as paichongding in China) is known from WO07/101369. M.22B.1 is described in CN10171577 and M.22B.2 in CN102126994. Phthalamides M.28.1 and M.28.2 are known from WO07/101540. M.28.3 is described in WO05/077934. M.28.4 is described in WO07/043677. M.28.5a) to M.28.5d) and M.28.5h) are described in WO07/006670, WO13/024009 and WO13/024010, M.28.5i) is described in WO11/085575, M.28.5j) in WO08/134969, M.28.5k) in US2011/046186 and M.28.51) in WO2012/034403. M.28.6 can be found in WO12/034472. M.29.3 is known from WO06/089633 and M.29.4 from WO08/067911. M.29.5 is described in WO06/043635, and biological control agents on the basis of bacillus firmus are described in WO09/124707. M.29.6a) to M.29.6i) listed under M.29.6 are described in WO12/029672, and M.29.6j) and M.29.6k) in WO13/129688. M.29.8 is known from WO13/055584. M.29.9.a) is described in WO13/050317. M.29.9.b) is described in WO2014/126208. M.29.10 is known from WO10/060379. Broflanilide and M.29.11.b) to M.29.11.h) are described in WO10/018714, and M.29.11i) to M.29.11.p) in WO10/127926. M.29.12.a) to M.29.12.c) are known from WO10/006713, M.29.12.d) and M.29.12.e) are known from WO12/000896, and M.29.12.f) to M.29.12.m) from WO10/129497. M.29.14a) and M.29.14b) are known from WO07/101369. M.29.16.a) to M.29.16h) are described in WO10/034737, WO12/084670, and WO12/143317, respectively, and M.29.16i) and M.29.16j) are described in US 61/891437. M.29.17a) to M.29.17.j) are described in WO15/038503. M.29.18a) to M.29.18d) are described in US2014/0213448. M.29.19 is described in WO14/036056. M.29.20 is known from WO14/090918.

The following list of fungicides, in conjunction with which the compounds of the present invention can be used, is intended to illustrate the possible combinations but does not limit them:

### A) Respiration Inhibitors

Inhibitors of complex III at Q_{O} site (e. g. strobilurins): azoxystrobin (A.1.1), coumethoxystrobin (A.1.2), coumoxystrobin (A.1.3), dimoxystrobin (A.1.4), enestroburin (A.1.5), fenaminstrobin (A.1.6), fenoxystrobin/flufenoxystrobin (A.1.7), fluoxastrobin (A.1.8), kresoxim-methyl (A.1.9), mandestrobin (A.1.10), metominostrobin (A.1.11), orysastrobin (A.1.12), picoxy.strobin (A.1.13), pyraclostrobin (A.1.14), pyrametostrobin (A.1.15), pyraoxystrobin (A.1.16), trifloxystrobin (A.1.17), 2-(2-(3-(2,6-dichlorophenyl)-1-methyl-allylideneaminooxymethyl)-phenyl)-2-methoxyimino-N-methyl-acetamide (A.1.18), pyribencarb (A.1.19), triclopyricarb/chlorodincarb (A.1.20), famoxadone (A.1.21), fenamidone (A.1.21), methyl-N-[2-[(1,4-dimethyl-5-phenylpyrazol-3-yl)oxylmethyl]phenyl]-N-methoxy-carbamate (A.1.22), 1-[3-chloro-2-[[1-(4-chlorophenyl)-1H-pyrazol-3-yl]oxymethyl]phenyl]-4-methyl-tetrazol-5-one (A.1.23), 1-[3-bromo-2-[[1-(4-chlorophenyl)pyrazol-3-yl]oxymethyl]phenyl]-4-methyl-tetrazol-5-one (A.1.24), 1-[2-[[1-(4-chlorophenyl)pyrazol-3-yl]oxymethyl]-3-methyl-phenyl]-4-methyl-tetrazol-5-one (A.1.25), 1-[2-[[1-(4-chlorophenyl)pyrazol-3-yl]oxymethyl]-3-fluoro-phenyl]-4-methyl-tetrazol-5-one (A.1.26), 1-[2-[[1-(2,4-dichlorophenyl)pyrazol-3-yl]oxymethyl]-3-fluoro-phenyl]-4-methyl-tetrazol-5-one (A.1.27), 1-[2-[[4-(4-chlorophenyl)thiazol-2-yl]oxymethyl]-3-methyl-phenyl]-4-methyl-tetrazol-5-one (A.1.28), 1-[3-chloro-2-[[4-(p-tolyl)thiazol-2-yl]oxymethyl]phenyl]-4-methyl-tetrazol-5-one (A.1.29), 1-[3-cyclopropyl-2-[[2-methyl-4-(1-methylpyrazol-3-yl)phenoxy]methyl]phenyl]-4-methyl-tetrazol-5-one (A.1.30), 1-[3-(difluoromethoxy)-2-[[2-methyl-4-(1-methyl)pyrazol-3-yl)phenoxy]methyl]phenyl]-4-methyl-tetrazol-5-one (A.1.31), 1-methyl-4-[3-methyl-2-[[2-methyl-4-(1-methylpyrazol-3-yl)phenoxy]methyl]phenyl]tetrazol-5-one (A.1.32), 1-methyl-4-[3-methyl-2-[[1-[3-(trifluoromethyl)phenyl]-ethylideneamino]oxymethyl]phenyl]tetrazol-5-one (A.1.33), (Z,2E)-5-[1-(2,4-dichlorophenyl)pyrazol-3-yl]-oxy-2-methoxyimino-N,3-dimethyl-pent-3-enamide (A.1.34), (Z,2E)-5-[1-(4-chlorophenyl)pyrazol-3-yl]oxy-2-methoxyimino-N,3-dimethyl-pent-3-enamide (A.1.35), (Z,2E)-5-[1-(4-chloro-2-fluoro-phenyl)pyrazol-3-yl]oxy-2-methoxyimino-N,3-dimethyl-pent-3-enamide (A.1.36),

inhibitors of complex III at Qᵢ site: cyazofamid (A.2.1), amisulbrom (A.2.2), [(3S,6S,7R,8R)-8-benzyl-3-[(3-acetoxy-4-methoxy-pyridine-2-carbonyl)amino]-6-methyl-4,9-dioxo-1,5-dioxonan7-yl] 2-methylpropanoate (A.2.3), [(3S,6S,7R,8R)-8-benzyl-3-[[3-(acetoxymethoxy)-4-methoxy-pyridine-2-carbonyl]amino]-6-methyl-4,9-dioxo-1,5-dioxonan-7-yl] 2-methylpropanoate (A.2.4), [(3S,6S,7R,8R)-8-benzyl-3-[(3-isobutoxycarbonyloxy-4-methoxy-pyridine-2-carbonyl)amino]-6-methyl-4,9-dioxo-1,5-dioxonan-7-yl] 2-methylpropanoate (A.2.5), [(3S,6S,7R,8R)-8-benzyl-3-[[3-(1,3-benzodioxol-5-ylmethoxy)-4-methoxy-pyridine-2-carbonyl]amino]-6-methyl-4,9-dioxo-1,5-dioxonan-7-yl] 2-methylpropanoate (A.2.6); (3S,6S,7R,8R)-3-[[(3-hydroxy-4-methoxy-2-pyridinyl)carbonyl]amino]-6-methyl-4,9-dioxo-8-(phenylmethyl)-1,5-dioxonan-7-yl2-methyl-propanoate (A.2.7), (3S,6S,7R,8R)-8-benzyl-3-[3-[(isobutyryloxy)methoxy]-4-methoxypicolinamido]-6-methyl-4,9-dioxo-1,5-dioxonan-7-yl isobutyrate (A.2.8);

inhibitors of complex II (e. g. carboxamides): benodanil (A.3.1), benzovindiflupyr (A.3.2), bixafen (A.3.3), boscalid (A.3.4), carboxin (A.3.5), fenfuram (A.3.6), fluopyram (A.3.7), flutolanil (A.3.8), fluxapyroxad (A.3.9), furametpyr (A.3.10), isofetamid (A.3.11), isopyrazam (A.3.12), mepronil (A.3.13), oxycarboxin (A.3.14), penflufen (A.3.14), penthiopyrad (A.3.15), sedaxane (A.3.16), tecloftalam (A.3.17), thifluzamide (A.3.18), N-(4′-trifluoromethylthiobiphenyl-2-yl)-3-difluoromethyl-1-methyl-1H-pyrazole-4-carboxamide (A.3.19), N-(2-(1,3,3-trimethyl-butyl)-phenyl)-1,3-dimethyl-5-fluoro-1H-pyrazole-4-carboxamide (A.3.20), 3-(difluoromethyl)-1-methyl-N-(1,1,3-trimethylindan-4-yl)pyrazole-4-carboxamide (A.3.21), 3-(trifluoromethyl)-1-methyl-N-(1,1,3-trimethylindan-4-yl)pyrazole-4-carboxamide (A.3.22), 1,3-dimethyl-N-(1,1,3-trimethyl-indan-4-yl)pyrazole-4-carboxamide (A.3.23), 3-(trifluoromethyl)-1,5-dimethyl-N-(1,1,3-trimethylindan-4-yl)pyrazole-4-carboxamide (A.3.24), 1,3,5-trimethyl-N-(1,1,3-trimethylindan-4-yl)pyrazole-4-carboxamide (A.3.25), N-(7-fluoro-1,1,3-trimethyl-indan-4-yl)-1,3-dimethyl-pyrazole-4-carboxamide (A.3.26), N-[2-(2,4-dichlorophenyl)-2-methoxy-1-methyl-ethyl]-3-(difluoromethyl)-1-methyl-pyrazole-4-carboxamide (A.3.27);

other respiration inhibitors (e. g. complex I, uncouplers): diflumetorim (A.4.1), (5,8-difluoro-quinazolin-4-yl)-{2-[2-fluoro-4-(4-trifluoromethylpyridin-2-yloxy)-phenyl]-ethyl}-amine (A.4.2); nitrophenyl derivates: binapacryl (A.4.3), dinobuton (A.4.4), dinocap (A.4.5), fluazinam (A.4.6); ferimzone (A.4.7); organometal compounds: fentin salts, such as fentin-acetate (A.4.8), fentin chloride (A.4.9) or fentin hydroxide (A.4.10); ametoctradin (A.4.11); and silthiofam (A.4.12);

### B) Sterol Biosynthesis Inhibitors (SBI Fungicides)

C14 demethylase inhibitors (DMI fungicides): triazoles: azaconazole (B.1.1), bitertanol (B.1.2), bromuconazole (B.1.3), cyproconazole (B.1.4), difenoconazole (B.1.5), diniconazole (B.1.6), diniconazole-M (B.1.7), epoxiconazole (B.1.8), fenbuconazole (B.1.9), fluquinconazole (B.1.10), flusilazole (B.1.11), flutriafol (B.1.12), hexaconazole (B.1.13), imibenconazole (B.1.14), ipconazole (B.1.15), metconazole (B.1.17), myclobutanil (B.1.18), oxpoconazole (B.1.19), paclobutrazole (B.1.20), penconazole (B.1.21), propiconazole (B.1.22), prothioconazole (B.1.23), simeconazole (B.1.24), tebuconazole (B.1.25), tetraconazole (B.1.26), triadimefon (B.1.27), triadimenol (B.1.28), triticonazole (B.1.29), uniconazole (B.1.30), 1-[rel-(2S,3R)-3-(2-chlorophenyl)-2-(2,4-difluorophenyl)-oxiranylmethyl]-5-thiocyanato-1H-[1,2,4]triazolo (B.1.31), 2-[rel(2S;3R)-3-(2-chlorophenyl)-2-(2,4-difluorophenyl)-oxiranylmethyl]-2H-[1,2,4]triazole-3-thiol (B.1.32), 2-[2-chloro-4-(4-chlorophenoxy)phenyl]-1-(1,2,4-triazol-1-yl)pentan-2-ol (B.1.33), 1-[4-(4-chlorophenoxy)-2-(trifluoromethyl)phenyl]-1-cyclopropyl-2-(1,2,4-triazol-1-yl)ethanol (B.1.34), 2-[4-(4-chlorophenoxy)-2-(trifluoromethyl)phenyl]-1-(1,2,4-triazol-1-yl)butan-2-ol (B.1.35), 2-[2-chloro-4-(4-chlorophenoxy)phenyl]-1-(1,2,4-triazol-1-yl)butan-2-ol (B.1.36), 2-[4-(4-chloro-phenoxy)-2-(trifluoromethyl)phenyl]-3-methyl-1-(1,2,4-triazol-1-yl)butan-2-ol (B.1.37), 2-[4-(4-chlorophenoxy)-2-(trifluoromethyl)phenyl]-1-(1,2,4-triazol-1-yl)propan-2-ol (B.1.38), 2-[2-chloro-4-(4-chlorophenoxy)phenyl]-3-methyl-1-(1,2,4-triazol-1-yl)butan-2-ol (B.1.39), 2-[4-(4-chloro-phenoxy)-2-(trifluoromethyl)phenyl]-1-(1,2,4-triazol-1-yl)pentan-2-ol (B.1.40), 2-[4-(4-fluorophen-oxy)-2-(trifluoromethyl)phenyl]-1-(1,2,4-triazol-1-yl)propan-2-ol (B.1.41), 2-[2-chloro-4-(4-chlorophenoxy)phenyl]-1-(1,2,4- triazol-1-yl)pent-3-yn-2-ol (B.1.51); imidazoles: imazalil (B.1.42), pefurazoate (B.1.43), prochloraz (B.1.44), triflumizol (B.1.45); pyrimidines, pyridines and piperazines: fenarimol (B.1.46), nuarimol (B.1.47), pyrifenox (B.1.48), triforine (B.1.49), [3-(4-chloro-2-fluoro-phenyl)-5-(2,4-difluorophenyl)isoxazol- 4-yl]-(3-pyridyl)methanol (B.1.50);

Delta14-reductase inhibitors: aldimorph (B.2.1), dodemorph (B.2.2), dodemorph-acetate (B.2.3), fenpropimorph (B.2.4), tridemorph (B.2.5), fenpropidin (B.2.6), piperalin (B.2.7), spiroxamine (B.2.8);

Inhibitors of 3-keto reductase: fenhexamid (B.3.1);

### C) Nucleic Acid Synthesis Inhibitors

phenylamides or acyl amino acid fungicides: benalaxyl (C.1.1), benalaxyl-M (C.1.2), kiralaxyl (C.1.3), metalaxyl (C.1.4), metalaxyl-M (mefenoxam, C.1.5), ofurace (C.1.6), oxadixyl (C.1.7);

others: hymexazole (C.2.1), octhilinone (C.2.2), oxolinic acid (C.2.3), bupirimate (C.2.4), 5-fluorocytosine (C.2.5), 5-fluoro-2-(p-tolylmethoxy)pyrimidin-4-amine (C.2.6), 5-fluoro-2-(4-fluorophenylmethoxy)pyrimidin-4-amine (C.2.7);

### D) Inhibitors of Cell Division and Cytoskeleton

tubulin inhibitors, such as benzimidazoles, thiophanates: benomyl (D1.1), carbendazim (D1.2), fuberidazole (D1.3), thiabendazole (D1.4), thiophanate-methyl (D1.5); triazolopyrimidines: 5-chloro-7-(4-methylpiperidin-1-yl)-6-(2,4,6-trifluorophenyl)-[1,2,4]triazolo[1,5-a]pyrimidine (D1.6);

other cell division inhibitors: diethofencarb (D2.1), ethaboxam (D2.2), pencycuron (D2.3), fluopicolide (D2.4), zoxamide (D2.5), metrafenone (D2.6), pyriofenone (D2.7);

### E) Inhibitors of Amino Acid and Protein Synthesis

methionine synthesis inhibitors (anilino-pyrimidines): cyprodinil (E.1.1), mepanipyrim (E.1.2), pyrimethanil (E.1.3);

protein synthesis inhibitors: blasticidin-S (E.2.1), kasugamycin (E.2.2), kasugamycin hydrochloride-hydrate (E.2.3), mildiomycin (E.2.4), streptomycin (E.2.5), oxytetracyclin (E.2.6), polyoxine (E.2.7), validamycin A (E.2.8);

### F) Signal Transduction Inhibitors

MAP/histidine kinase inhibitors: fluoroimid (F.1.1), iprodione (F.1.2), procymidone (F.1.3), vinclozolin (F.1.4), fenpiclonil (F.1.5), fludioxonil (F.1.6);

G protein inhibitors: quinoxyfen (F.2.1);

### G) Lipid and Membrane Synthesis Inhibitors

Phospholipid biosynthesis inhibitors: edifenphos (G.1.1), iprobenfos (G.1.2), pyrazophos (G.1.3), isoprothiolane (G.1.4);

lipid peroxidation: dicloran (G.2.1), quintozene (G.2.2), tecnazene (G.2.3), tolclofos-methyl (G.2.4), biphenyl (G.2.5), chloroneb (G.2.6), etridiazole (G.2.7);

phospholipid biosynthesis and cell wall deposition: dimethomorph (G.3.1), flumorph

(G.3.2), mandipropamid (G.3.3), pyrimorph (G.3.4), benthiavalicarb (G.3.5), iprovalicarb (G.3.6), valifenalate (G.3.7) and N-(1-(1-(4-cyano-phenypethanesulfonyl)-but-2-yl) carbamic acid-(4-fluorophenyl) ester (G.3.8);

compounds affecting cell membrane permeability and fatty acides: propamocarb (G.4.1);

fatty acid amide hydrolase inhibitors: oxathiapiprolin (G.5.1), 2-{3-[2-(1-{[3,5-bis(difluoromethyl-1H-pyrazol-1-yl]acetyl}piperidin-4-yl)-1,3-thiazol-4-yl]-4,5-dihydro-1,2-oxazol-5-yl}phenyl methanesulfonate (G.5.2), 2-{3-[2-(1-{[3,5-bis(difluoromethyl)-1H-pyrazol-1-yl]acetyl}piperidin-4-yl) 1,3-thiazol-4-yl]-4,5-dihydro-1,2-oxazol-5-yl}-3-chlorophenyl methanesulfonate (G.5.3);

### H) Inhibitors with Multi Site Action

inorganic active substances: Bordeaux mixture (H.1.1), copper acetate (H.1.2), copper hydroxide (H.1.3), copper oxychloride (H.1.4), basic copper sulfate (H.1.5), sulfur (H.1.6);

thio- and dithiocarbamates: ferbam (H.2.1), mancozeb (H.2.2), maneb (H.2.3), metam (H.2.4), metiram (H.2.5), propineb (H.2.6), thiram (H.2.7), zineb (H.2.8), ziram (H.2.9);

organochlorine compounds (e. g. phthalimides, sulfamides, chloronitriles): anilazine (H.3.1), chlorothalonil (H.3.2), captafol (H.3.3), captan (H.3.4), folpet (H.3.5), dichlofluanid (H.3.6), dichlorophen (H.3.7), hexachlorobenzene (H.3.8), pentachlorphenole (H.3.9) and its salts, phthalide (H.3.10), tolylfluanid (H.3.11), N-(4-chloro-2-nitro-phenyl)-N-ethyl-4-methyl-benzenesulfonamide (H.3.12);

guanidines and others: guanidine (H.4.1), dodine (H.4.2), dodine free base (H.4.3), guazatine (H.4.4), guazatine-acetate (H.4.5), iminoctadine (H.4.6), iminoctadine-triacetate (H.4.7), iminoctadine-tris(albesilate) (H.4.8), dithianon (H.4.9), 2,6-dimethyl-1H,5H-[1,4]dithiino[2,3-c:5,6-c′]dipyrrole-1,3,5,7(2H,6H)-tetraone (H.4.10);

### I) Cell Wall Synthesis Inhibitors

inhibitors of glucan synthesis: validamycin (I.1.1), polyoxin B (I.1.2);

melanin synthesis inhibitors: pyroquilon (I.2.1), tricyclazole (I.2.2), carpropamid (I.2.3), dicyclomet (I 2.4), fenoxanil (I 2.5);

### J) Plant Defence Inducers

acibenzolar-S-methyl (J.1.1), probenazole (J.1.2), isotianil (J.1.3), tiadinil (J.1.4), prohexadione-calcium (J.1.5); phosphonates: fosetyl (J.1.6), fosetyl- aluminum (J.1.7), phosphorous acid and its salts (J.1.8), potassium or sodium bicarbonate (J.1.9);

### K) Unknown Mode Of Action

bronopol (K.1.1), chinomethionat (K.1.2), cyflufenamid (K.1.3), cymoxanil (K.1.4), dazomet (K.1.5), debacarb (K.1.6), diclomezine (K.1.7), difenzoquat (K.1.8), difenzoquat-methylsulfate (K.1.9), diphenylamin (K.1.10), fenpyrazamine (K.1.11), flumetover (K.1.12), flusulfamide (K.1.13), flutianil (K.1.14), methasulfocarb (K.1.15), nitrapyrin (K.1.16), nitrothal-isopropyl (K.1.18), oxathiapiprolin (K.1.19), tolprocarb (K.1.20), oxin-copper (K.1.21), proquinazid (K.1.22), tebufloquin (K.1.23), tecloftalam (K.1.24), triazoxide (K.1.25), 2-butoxy-6-iodo-3-propylchromen-4-one (K.1.26), 2-[3,5-bis(difluoromethyl)-1H-pyrazol-1-yl]-1-[4-(4-{5-[2-(prop-2-yn-1-yloxy)phenyl]-4,5-dihydro-1,2-oxazol-3-yl}-1,3-thiazol-2-yl)piperidin-1-yl]ethanone (K.1.27), 2-[3,5-bis(difluoromethyl)-1H-pyrazol-1-yl]-1-[4-(4-{5-[2-fluoro-6-(prop-2-yn-1-yl-oxy)phenyl]-4,5-dihydro-1,2-oxazol-3-yl}-1,3-thiazol-2-yl)piperidin-1-yl]ethanone (K.1.28), 2-[3,5-bis(difluoromethyl)-1H-pyrazol-1-yl]-1-[4-(4-{5-[2-chloro-6-(prop-2-yn-1- yloxy)phenyl]-4,5-dihydro-1,2-oxazol-3-yl}-1,3-thiazol-2-yl)piperidin-1-yl]ethanone (K.1.29), N-(cyclo-propylmethoxyimino-(6-difluoro-methoxy-2,3-difluoro-phenyl)-methyl)-2-phenyl acetamide (K.1.30), N′-(4-(4-chloro-3-trifluoromethyl-phenoxy)-2,5-dimethyl-phenyl)-N-ethyl-N-methyl formamidine (K.1.31), N′-(4-(4-fluoro-3-trifluoromethyl-phenoxy)-2,5-dimethyl-phenyl)-N-ethyl-N-methyl formamidine (K.1.32), N′-(2-methyl-5-trifluoromethyl-4-(3-trimethylsilanyl-propoxy)-phenyl)-N-ethyl-N-methyl formamidine (K.1.33), N′-(5- difluoromethyl-2-methyl-4-(3-tri-methylsilanyl-propoxy)-phenyl)-N-ethyl-N-methyl formamidine (K.1.34), methoxy-acetic acid 6-tert-butyl-8-fluoro-2,3- dimethyl-quinolin-4-yl ester (K.1.35), 3-[5-(4-methylphenyl)-2,3-dimethyl-isoxazolidin-3-yl]-pyridine (K.1.36), 3-[5-(4-chloro-phenyl)-2,3-dimethyl-isoxazolidin-3-yl]-pyridine (pyrisoxazole) (K.1.37), N-(6-methoxy-pyridin-3-yl) cyclopropanecarboxylic acid amide (K.1.38), 5-chloro-1-(4,6-dimethoxy-pyrimidin-2-yl -2-methyl-1H-benzoimidazole (K.1.39), 2-(4-chloro-phenyl)-N-[4-(3,4-dimethoxy-phenyl)-isoxazol-5-yl]-2-prop-2-ynyloxy-acetamide, ethyl (Z)-3-amino-2-cyano-3-phenyl-prop-2-enoate (K.1.40), picarbutrazox (K.1.41), pentyl N-[6-[[(Z)-[(1-methyltetrazol-5-yl)-phenyl-methylene]amino]oxymethyl]-2-pyridyl]carbamate (K.1.42), 2-[2-[(7,8-difluoro-2-methyl-3-quinolyl)oxy]-6-fluoro-phenyl]propan-2-ol (K.1.43), 2-[2-fluoro-6-[(8-fluoro-2-methyl-3-quinolyl)oxy]phen-yl]propan-2-ol (K.1.44), 3-(5-fluoro-3,3,4,4-tetramethyl-3,4-dihydroisoquinolin-1-yl)quinoline (K.1.45), 3-(4,4-difluoro-3,3-dimethyl-3,4-dihydroisoquinolin-1-yl)quinoline (K.1.46), 3-(4,4,5-trifluoro-3,3- dimethyl-3,4-dihydroisoquinolin-1-yl)quinoline (K.1.47), 9-fluoro-2,2-dimethyl-5-(3-quinolyl)-3H-1,4-benzoxazepine (K.1.48).

The fungicides described by common names, their preparation and their activity e.g. against harmful fungi is known (cf.: http://www.alanwood.net/pesticides/); these substances are commercially available.

The fungicides described by IU PAC nomenclature, their preparation and their pesticidal activity is also known (cf. Can. J. Plant Sci. 48(6), 587-94, 1968; EP-A 141 317; EP-A 152 031; EP-A 226 917; EP-A 243 970; EP-A 256 503; EP-A 428 941; EP-A 532 022; EP-A 1 028 125; EP-A 1 035 122; EP-A 1 201 648; EP-A 1 122 244, JP 2002316902; DE 19650197; DE 10021412; DE 102005009458; U.S. Pat. Nos. 3,296,272; 3,325,503; WO 98/46608; WO 99/14187; WO 99/24413; WO 99/27783; WO 00/29404; WO 00/46148; WO 00/65913; WO 01/54501; WO 01/56358; WO 02/22583; WO 02/40431; WO 03/10149; WO 03/11853; WO 03/14103; WO 03/16286; WO 03/53145; WO 03/61388; WO 03/66609; WO 03/74491; WO 04/49804; WO 04/83193; WO 05/120234; WO 05/123689; WO 05/123690; WO 05/63721; WO 05/87772; WO 05/87773;

WO 06/15866; WO 06/87325; WO 06/87343; WO 07/82098; WO 07/90624, WO 11/028657, WO2012/168188, WO 2007/006670, WO 2011/77514; WO13/047749, WO 10/069882, WO 13/047441, WO 03/16303, WO 09/90181, WO 13/007767, WO 13/010862, WO 13/127704, WO 13/024009, WO 13/024010 and WO 13/047441, WO 13/162072, WO 13/092224, WO 11/135833).

Suitable mixing partners for the compounds of the present invention also include biopesticides.

Biopesticides have been defined as a form of pesticides based on micro-organisms (bacteria, fungi, viruses, nematodes, etc.) or natural products (compounds, such as metabolites, proteins, or extracts from biological or other natural sources) (U.S. Environmental Protection Agency: http://www.epa.gov/pesticides/biopesticides/). Biopesticides fall into two major classes, microbial and biochemical pesticides:

(1) Microbial pesticides consist of bacteria, fungi or viruses (and often include the metabolites that bacteria and fungi produce). Entomopathogenic nematodes are also classified as microbial pesticides, even though they are multi-cellular.

(2) Biochemical pesticides are naturally occurring substances or or structurally-similar and functionally identical to a naturally-occurring substance and extracts from biological sources that control pests or provide other crop protection uses as defined below, but have non-toxic mode of actions (such as growth or developmental regulation, attractents, repellents or defence activators (e.g. induced resistance) and are relatively non-toxic to mammals.

Biopesticides for use against crop diseases have already established themselves on a variety of crops. For example, biopesticides already play an important role in controlling downy mildew diseases. Their benefits include: a 0-Day Pre-Harvest Interval, the ability to use under moderate to severe disease pressure, and the ability to use in mixture or in a rotational program with other registered pesticides.

A major growth area for biopesticides is in the area of seed treatments and soil amendments. Biopesticidal seed treatments are e.g. used to control soil borne fungal pathogens that cause seed rots, damping-off, root rot and seedling blights. They can also be used to control internal seed borne fungal pathogens as well as fungal pathogens that are on the surface of the seed. Many biopesticidal products also show capacities to stimulate plant host defenses and other physiological processes that can make treated crops more resistant to a variety of biotic and abiotic stresses or can regulate plant growth. Many biopesticidal products also show capacities to stimulate plant health, plant growth and/or yield enhancing activity.

The invention also relates to agrochemical compositions comprising an auxiliary and at least one compound of the present invention or a mixture thereof.

An agrochemical composition comprises a pesticidally effective amount of a compound of the present invention or a mixture thereof. The term “pesticidally effective amount” is defined below.

The compounds of the present invention or the mixtures thereof can be converted into customary types of agro-chemical compositions, e. g. solutions, emulsions, suspensions, dusts, powders, pastes, granules, pressings, capsules, and mixtures thereof. Examples for composition types are suspensions (e.g. SC, OD, FS), emulsifiable concentrates (e.g. EC), emulsions (e.g. EW, EO, ES, ME), capsules (e.g. CS, ZC), pastes, pastilles, wettable powders or dusts (e.g. WP, SP, WS, DP, DS), pressings (e.g. BR, TB, DT), granules (e.g. WG, SG, GR, FG, GG, MG), insecticidal articles (e.g. LN), as well as gel formulations for the treatment of plant propagation materials such as seeds (e.g. GF). These and further compositions types are defined in the “Catalogue of pesticide formulation types and international coding system”, Technical Monograph No. 2, 6th Ed. May 2008, CropLife International.

The compositions are prepared in a known manner, such as described by Mollet and Grubemann, Formulation technology, Wiley VCH, Weinheim, 2001; or Knowles, New developments in crop protection product formulation, Agrow Reports DS243, T&F Informa, London, 2005.

Examples for suitable auxiliaries are solvents, liquid carriers, solid carriers or fillers, surfactants, dispersants, emulsifiers, wetters, adjuvants, solubilizers, penetration enhancers, protective colloids, adhesion agents, thickeners, humectants, repellents, attractants, feeding stimulants compatibilizers, bactericides, anti-freezing agents, anti-foaming agents, colorants, tackifiers and binders.

Suitable solvents and liquid carriers are water and organic solvents, such as mineral oil fractions of medium to high boiling point, e.g. kerosene, diesel oil; oils of vegetable or animal origin; aliphatic, cyclic and aromatic hydrocarbons, e. g. toluene, paraffin, tetrahydronaphthalene, alkylated naphthalenes; alcohols, e.g. ethanol, propanol, butanol, benzylalcohol, cyclo-hexanol; glycols; DMSO; ketones, e.g. cyclohexanone; esters, e.g. lactates, carbonates, fatty acid esters, gamma-butyrolactone; fatty acids; phosphonates; amines; amides, e.g. N-methylpyrrolidone, fatty acid dimethylamides; and mixtures thereof.

Suitable solid carriers or fillers are mineral earths, e.g. silicates, silica gels, talc, kaolins, limestone, lime, chalk, clays, dolomite, diatomaceous earth, bentonite, calcium sulfate, magnesium sulfate, magnesium oxide; polysaccharide powders, e.g. cellulose, starch; fertilizers, e.g. ammonium sulfate, ammonium phosphate, ammonium nitrate, ureas; products of vegetable origin, e.g. cereal meal, tree bark meal, wood meal, nutshell meal, and mixtures thereof.

Suitable surfactants are surface-active compounds, such as anionic, cationic, nonionic and amphoteric surfactants, block polymers, polyelectrolytes, and mixtures thereof. Such surfactants can be used as emusifier, dispersant, solubilizer, wetter, penetration enhancer, protective colloid, or adjuvant. Examples of surfactants are listed in McCutcheon's, Vol.1: Emulsifiers & Detergents, McCutcheon's Directories, Glen Rock, USA, 2008 (International Ed. or North American Ed.).

Suitable anionic surfactants are alkali, alkaline earth or ammonium salts of sulfonates, sulfates, phosphates, carboxylates, and mixtures thereof. Examples of sulfonates are alkylaryl-sulfonates, diphenylsulfonates, alpha-olefin sulfonates, lignine sulfonates, sulfonates of fatty acids and oils, sulfonates of ethoxylated alkylphenols, sulfonates of alkoxylated arylphenols, sulfonates of condensed naphthalenes, sulfonates of dodecyl- and tridecylbenzenes, sulfonates of naphthalenes and alkyhnaphthalenes, sulfosuccinates or sulfosuccinamates. Examples of sulfates are sulfates of fatty acids and oils, of ethoxylated alkylphenols, of alcohols, of ethoxylated alcohols, or of fatty acid esters. Examples of phosphates are phosphate esters. Examples of carboxylates are alkyl carboxylates, and carboxylated alcohol or alkylphenol ethoxylates.

Suitable nonionic surfactants are alkoxylates, N-subsituted fatty acid amides, amine oxides, esters, sugar-based surfactants, polymeric surfactants, and mixtures thereof. Examples of alkoxylates are compounds such as alcohols, alkylphenols, amines, amides, arylphenols, fatty acids or fatty acid esters which have been alkoxylated with 1 to 50 equivalents. Ethylene oxide and/or propylene oxide may be employed for the alkoxylation, preferably ethylene oxide. Examples of N-subsititued fatty acid amides are fatty acid glucamides or fatty acid alkanolamides. Examples of esters are fatty acid esters, glycerol esters or monoglycerides. Examples of sugar-based surfactants are sorbitans, ethoxylated sorbitans, sucrose and glucose esters or alkylpolyglucosides. Examples of polymeric surfactants are homo- or copolymers of vinylpyrrolidone, vinylalcohols, or vinylacetate.sss

Suitable cationic surfactants are quaternary surfactants, for example quaternary ammonium compounds with one or two hydrophobic groups, or salts of long-chain primary amines. Suitable amphoteric surfactants are alkylbetains and imidazolines. Suitable block polymers are block polymers of the A-B or A-B-A type comprising blocks of polyethylene oxide and polypropylene oxide, or of the A-B-C type comprising alkanol, polyethylene oxide and polypropylene oxide. Suitable polyelectrolytes are polyacids or polybases. Examples of polyacids are alkali salts of polyacrylic acid or polyacid comb polymers. Examples of polybases are polyvinylamines or polyethyleneamines.

Suitable adjuvants are compounds, which have a neglectable or even no pesticidal activity themselves, and which improve the biological performance of the compounds of the present invention on the target. Examples are surfactants, mineral or vegetable oils, and other auxilades. Further examples are listed by Knowles, Adjuvants and additives, Agrow Reports DS256, T&F Informa UK, 2006, chapter 5.

Suitable thickeners are polysaccharides (e.g. xanthan gum, carboxymethylcellulose), anorganic clays (organically modified or unmodified), polycarboxylates, and silicates.

Suitable bactericides are bronopol and isothiazolinone derivatives such as alkylisothiazolinones and benzisothiazolinones.

Suitable anti-freezing agents are ethylene glycol, propylene glycol, urea and glycerin.

Suitable anti-foaming agents are silicones, long chain alcohols, and salts of fatty acids.

Suitable colorants (e.g. in red, blue, or green) are pigments of low water solubility and watersoluble dyes. Examples are inorganic colorants (e.g. iron oxide, titan oxide, iron hexacyanoferrate) and organic colorants (e.g. alizarin-, azo- and phthalocyanine colorants).

Suitable tackifiers or binders are polyvinylpyrrolidons, polyvinylacetates, polyvinyl alcohols, polyacrylates, biological or synthetic waxes, and cellulose ethers.

Examples for composition types and their preparation are:

### i) Water-Soluble Concentrates (SL, LS)

10-60 wt % of a compound I according to the invention and 5-15 wt % wetting agent (e.g. alcohol alkoxylates) are dissolved in water and/or in a water-soluble solvent (e.g. alcohols) up to 100 wt %. The active substance dissolves upon dilution with water.

### ii) Dispersible Concentrates (DC)

5-25 wt % of a compound I according to the invention and 1-10 wt % dispersant (e. g. polyvinylpyrrolidone) are dissolved in up to 100 wt % organic solvent (e.g. cyclohexanone). Dilution with water gives a dispersion.

### iii) Emulsifiable Concentrates (EC)

15-70 wt % of a compound I according to the invention and 5-10 wt % emulsifiers (e.g. calcium dodecylbenzenesulfonate and castor oil ethoxylate) are dissolved in up to 100 wt % water-insoluble organic solvent (e.g. aromatic hydrocarbon). Dilution with water gives an emulsion.

### iv) Emulsions (EW, EO, ES)

5-40 wt % of a compound I according to the invention and 1-10 wt % emulsifiers (e.g. calcium dodecylbenzenesulfonate and castor oil ethoxylate) are dissolved in 20-40 wt % water-insoluble organic solvent (e.g. aromatic hydrocarbon). This mixture is introduced into up to 100 wt % water by means of an emulsifying machine and made into a homogeneous emulsion. Dilution with water gives an emulsion.

### v) Suspensions (SC, OD, FS)

In an agitated ball mill, 20-60 wt % of a compound I according to the invention are comminuted with addition of 2-10 wt % dispersants and wetting agents (e.g. sodium lignosulfonate and alcohol ethoxylate), 0,1-2 wt % thickener (e.g. xanthan gum) and up to 100 wt % water to give a fine active substance suspension. Dilution with water gives a stable suspension of the active substance. For FS type composition up to 40 wt % binder (e.g. polyvinylalcohol) is added.

### vi) Water-Dispersible Granules And Water-Soluble Granules (WG, SG)

50-80 wt % of a compound I according to the invention are ground finely with addition of up to 100 wt % dispersants and wetting agents (e.g. sodium lignosulfonate and alcohol ethoxylate) and prepared as water-dispersible or water-soluble granules by means of technical appliances (e. g. extrusion, spray tower, fluidized bed). Dilution with water gives a stable dispersion or solution of the active substance.

### vii) Water-Dispersible Powders And Water-Soluble Powders (WP, SP, WS)

50-80 wt % of a compound I according to the invention are ground in a rotor-stator mill with addition of 1-5 wt % dispersants (e.g. sodium lignosulfonate), 1-3 wt % wetting agents (e.g. alcohol ethoxylate) and up to 100 wt % solid carrier, e.g. silica gel. Dilution with water gives a stable dispersion or solution of the active substance.

### viii) Gel (GW, GF)

In an agitated ball mill, 5-25 wt % of a compound I according to the invention are comminuted with addition of 3-10 wt % dispersants (e.g. sodium lignosulfonate), 1-5 wt % thickener (e.g. carboxymethylcellulose) and up to 100 wt % water to give a fine suspension of the active substance. Dilution with water gives a stable suspension of the active substance.

### ix) Microemulsion (ME)

5-20 wt % of a compound I according to the invention are added to 5-30 wt % organic solvent blend (e.g. fatty acid dimethylamide and cyclohexanone), 10-25 wt % surfactant blend (e.g. alkohol ethoxylate and arylphenol ethoxylate), and water up to 100%. This mixture is stirred for 1 h to produce spontaneously a thermodynamically stable microemulsion.

### x) Microcapsules (CS)

An oil phase comprising 5-50 wt % of a compound I according to the invention, 0-40 wt % water insoluble organic solvent (e.g. aromatic hydrocarbon), 2-15 wt % acrylic monomers (e.g. methylmethacrylate, methacrylic acid and a di- or triacrylate) are dispersed into an aqueous solution of a protective colloid (e.g. polyvinyl alcohol). Radical polymerization initiated by a radical initiator results in the formation of poly(meth)acrylate microcapsules. Alternatively, an oil phase comprising 5-50 wt % of a compound I according to the invention, 0-40 wt % water insoluble organic solvent (e.g. aromatic hydrocarbon), and an isocyanate monomer (e.g. diphenylmethene-4,4′-diisocyanatae) are dispersed into an aqueous solution of a protective colloid (e.g. polyvinyl alcohol). The addition of a polyamine (e.g. hexamethylenediamine) results in the formation of a polyurea microcapsule. The monomers amount to 1-10 wt %. The wt % relate to the total CS composition.

### xi) Dustable Powders (DP, DS)

1-10 wt % of a compound I according to the invention are ground finely and mixed intimately with up to 100 wt % solid carrier, e.g. finely divided kaolin.

### xii) Granules (GR, FG)

0.5-30 wt % of a compound I according to the invention is ground finely and associated with up to 100 wt % solid carrier (e.g. silicate). Granulation is achieved by extrusion, spray-drying or the fluidized bed.

xiii) Ultra-Low Volume Liquids (UL)

1-50 wt % of a compound I according to the invention are dissolved in up to 100 wt % organic solvent, e.g. aromatic hydrocarbon.

The compositions types i) to xi) may optionally comprise further auxiliaries, such as 0.1-1 wt % bactericides, 5-15 wt % anti-freezing agents, 0.1-1 wt % anti-foaming agents, and 0.1-1 wt % colorants.

The agrochemical compositions generally comprise between 0.01 and 95%, preferably between 0.1 and 90%, and most preferably between 0.5 and 75%, by weight of active sub-stance. The active substances are employed in a purity of from 90% to 100%, preferably from 95% to 100% (according to NMR spectrum).

Various types of oils, wetters, adjuvants, fertilizer, or micronutrients, and other pesticides (e.g. herbicides, insecticides, fungicides, growth regulators, safeners) may be added to the active substances or the compositions comprising them as premix or, if appropriate not until immediately prior to use (tank mix). These agents can be admixed with the compositions according to the invention in a weight ratio of 1:100 to 100:1, preferably 1:10 to 10:1.

The user applies the composition according to the invention usually from a predosage de-vice, a knapsack sprayer, a spray tank, a spray plane, or an irrigation system. Usually, the agrochemical composition is made up with water, buffer, and/or further auxiliaries to the desired application concentration and the ready-to-use spray liquor or the agrochemical composition according to the invention is thus obtained. Usually, 20 to 2000 liters, preferably 50 to 400 liters, of the ready-to-use spray liquor are applied per hectare of agricultural useful area.

According to one embodiment, individual components of the composition according to the invention such as parts of a kit or parts of a binary or ternary mixture may be mixed by the user himself in a spray tank and further auxiliaries may be added, if appropriate.

In a further embodiment, either individual components of the composition according to the invention or partially premixed components, e. g. components comprising compounds of the present invention and/or mixing partners as defined above, may be mixed by the user in a spray tank and further auxiliaries and additives may be added, if appropriate.

In a further embodiment, either individual components of the composition according to the invention or partially premixed components, e. g. components comprising compounds of the present invention and/or mixing partners as defined above, can be applied jointly (e.g. after tank mix) or consecutively.

The compounds of the present invention are suitable for use in treating or protecting animals against infestation or infection by parasites. Therefore, the present invention also relates to the use of a compound of the present invention for the manufacture of a medicament for the treatment or protection of animals against infestation or infection by parasites. Furthermore, the present invention relates to a method of treating or protecting animals against infestation and infection by parasites, which comprises orally, topically or parenterally administering or applying to the animals a parasiticidally effective amount of a compound of the present invention.

The present invention also relates to the non-therapeutic use of compounds of the present invention for treating or protecting animals against infestation and infection by parasites. Moreover, the present invention relates to a non-therapeutic method of treating or protecting animals against infestation and infection by parasites, which comprises applying to a locus a parasiticidally effective amount of a compound of the present invention.

The compounds of the present invention are further suitable for use in combating or controlling parasites in and on animals. Furthermore, the present invention relates to a method of combating or controlling parasites in and on animals, which comprises contacting the parasites with a parasitically effective amount of a compound of the present invention.

The present invention also relates to the non-therapeutic use of compounds of the present invention for controlling or combating parasites. Moreover, the present invention relates to a nontherapeutic method of combating or controlling parasites, which comprises applying to a locus a parasiticidally effective amount of a compound of the present invention.

The compounds of the present invention can be effective through both contact (via soil, glass, wall, bed net, carpet, blankets or animal parts) and ingestion (e.g. baits). Furthermore, the compounds of the present invention can be applied to any and all developmental stages.

The compounds of the present invention can be applied as such or in form of compositions comprising the compounds of the present invention.

The compounds of the present invention can also be applied together with a mixing partner, which acts against pathogenic parasites, e.g. with synthetic coccidiosis compounds, polyetherantibiotics such as Amprolium, Robenidin, Toltrazuril, Monensin, Salinomycin, Maduramicin, Lasalocid, Narasin or Semduramicin, or with other mixing partners as defined above, or in form of compositions comprising said mixtures.

The compounds of the present invention and compositions comprising them can be applied orally, parenterally or topically, e.g. dermally. The compounds of the present invention can be systemically or non-systemically effective.

The application can be carried out prophylactically, therapeutically or non-therapeutically. Furthermore, the application can be carried out preventively to places at which occurrence of the parasites is expected.

As used herein, the term “contacting” includes both direct contact (applying the compounds/compositions directly on the parasite, including the application directly on the animal or excluding the application directly on the animal, e.g. at it's locus for the latter) and indirect contact (applying the compounds/compositions to the locus of the parasite). The contact of the parasite through application to its locus is an example of a non-therapeutic use of the compounds of the present invention.

The term “locus” means the habitat, food supply, breeding ground, area, material or environment in which a parasite is growing or may grow outside of the animal.

As used herein, the term “parasites” includes endo- and ectoparasites. In some embodiments of the present invention, endoparasites can be preferred. In other embodiments, ectoparasites can be preferred. Infestations in warm-blooded animals and fish include, but are not limited to, lice, biting lice, ticks, nasal bots, keds, biting flies, muscoid flies, flies, myiasitic fly larvae, chiggers, gnats, mosquitoes and fleas.

The compounds of the present invention are especially useful for combating parasites of the following orders and species, respectively:

fleas (Siphonaptera), e.g. Ctenocephalides felis, Ctenocephalides canis, Xenopsylla cheopis, Pulex irritans, Tunga penetrans, and Nosopsyllus fasciatus; cockroaches (Blattaria-Blattodea), e.g. Blattella germanica, Blattella asahinae, Periplaneta americana, Periplaneta japonica, Periplaneta brunnea, Periplaneta fuligginosa, Periplaneta australasiae, and Blatta orientals, flies, mosquitoes (Diptera), e.g. Aedes aegypti, Aedes albopictus, Aedes vexans, Anastrepha ludens, Anopheles maculipennis, Anopheles crucians, Anopheles albimanus, Anopheles gambiae, Anopheles freeborni, Anopheles leucosphyrus, Anopheles minimus, Anopheles quadrimaculatus, Calliphora vicina, Chrysomya bezziana, Chrysomya hominivorax, Chrysomya macellaria, Chrysops discalis, Chrysops silacea, Chrysops atlanticus, Cochliomyia hominivorax, Cordylobia anthropophaga, Culicoides furens, Culex pipiens, Culex nigripalpus, Culex quinquefasciatus, Culex tarsalis, Culiseta inornata, Culiseta melanura, Dermatobia hominis, Fannia canicularis, Gasterophilus intestinalis, Glossina morsitans, Glossina palpalis, Glossina fuscipes, Glossina tachinoides, Haematobia irritans, Haplodiplosis equestris, Hippelates spp., Hypoderma lineata, Leptoconops torrens, Lucllia caprina, Lucilia cuprina, Lucllia sericata, Lycoria pectoralis, Mansonia spp., Musca domestica, Muscina stabulans, Oestrus ovis, Phlebotomus argentipes, Psorophora columbiae, Psorophora discolor, Prosimulium mixtum, Sarcophaga haemorrhoidalis, Sarcophaga sp., Simulium vittatum, Stomoxys calcitrans, Tabanus bovinus, Tabanus atratus, Tabanus lineola, and Tabanus similis; lice (Phthiraptera), e.g. Pediculus humanus capitis, Pediculus humanus corporis, Pthirus pubis, Haematopinus eurysternus, Haematopinus suis, Linognathus Bovicola bovis, Menopon gallinae, Menacanthus stramineus and Solenopotes capillatus; ticks and parasitic mites (Parasitiformes): ticks (Ixodida), e.g. Ixodes scapularis, Ixodes holocyclus, Ixodes pacificus, Rhiphicephalus sanguineus, Dermacentor andersoni, Dermacentor variabilis, Amblyomma americanum, Ambryomma maculatum, Ornithodorus hermsi, Ornithodorus turnicata and parasitic mites (Mesostigmata), e.g. Ornithonyssus bacoti and Dermanyssus gallinae; Actinedida (Prostigmata) and Acaridida (Astigmata), e.g. Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp., and Laminosioptes spp; Bugs (Heteropterida): Cimex lectularius, Cimex hemipterus, Reduvius senilis, Triatoma spp., Rhodnius ssp., Panstrongylus ssp., and Arilus critatus; Anoplurida, e.g. Haematopinus spp., Linognathus spp., Pediculus spp., Phtirus spp., and Solenopotes spp.; Mallophagida (suborders Arnblycerina and Ischnocerina), e.g. Trimenopon spp., Menopon spp., Trinoton spp., Bovicola spp., Werneckiella spp., Lepikentron spp., Trichodectes spp., and Felicola spp.; Roundworms Nematoda: Wipeworms and Trichinosis (Trichosyringida), e.g. Trichinellidae (Trichinella spp), (Trichuridae) Trichuris spp., Capillaria spp.; Rhabditida, e.g. Rhabditis spp., Strongyloides spp., Helicephalobus spp.; Strongylida, e.g. Strongylus spp., Ancylostoma spp., Necator americanus, Bunostomum spp. (Hookworm), Trichostrongylus spp., Haemonchus contortus, Ostertagia spp., Cooperia spp., Nematodirus spp., Dictyocaulus spp., Cyathostoma spp., Oesophagostomum spp., Stephanurus dentatus, Ollulanus spp., Chabertia spp., Stephanurus dentatus, Syngamus trachea, Ancylostoma spp., Uncinaria spp., Globocephalus spp., Necator spp., Metastrongylus spp., Muellerus capllaris, Protostrongylus spp., Angiostrongylus spp., Parelaphostrongylus spp., Aleurostrongylus abstrusus, and Dioctophyma renale; Intestinal roundworms (Ascaridida), e.g. Ascaris lumbricoides, Ascaris suum, Ascaridia galli, Parascaris equorum, Enterobius vermicularis(Threadworm), Toxocara canis, Toxascaris leonine, Skrjabinema spp., and Oxyuris equi; Camallanida, e.g. Dracunculus medinensis (guinea worm); Spirurida, e.g. Thelazia spp., Wuchereria spp., Brugia spp., Onchocerca spp., Dirofilari spp. a, Dipetalonema spp., Setaria spp., Elaeophora spp., Spirocerca lupi, and Habronema spp.; Thorny headed worms (Acanthocephala), e.g. Acanthocephalus spp., Macracanthorhynchus hirudinaceus and Oncicola spp.; Planarians (Plathelminthes): Flukes (Trematoda), e.g. Faciola spp., Fascioloides magna, Paragonimus spp., Dicrocoelium spp., Fasciolopsis buski, Clonorchis sinensis, Schistosoma spp., Trichobilharzia spp., Alaria alata, Paragonimus spp., and Nanocyetes spp.; Cercomeromorpha, in particular Cestoda (Tapeworms), e.g. Diphyllobothrium spp., Tenia spp., Echinococcus spp., Dipylidium caninum, Multiceps spp., Hymenolepis spp., Mesocestoides spp., Vampirolepis spp., Moniezia spp., Anoplocephala spp., Sirometra spp., Anoplocephala spp., and Hymenolepis spp.

As used herein, the term “animal” includes warm-blooded animals (including humans) and fish. Preferred are mammals, such as cattle, sheep, swine, camels, deer, horses, pigs, poultry, rabbits, goats, dogs and cats, water buffalo, donkeys, fallow deer and reindeer, and also in furbearing animals such as mink, chinchilla and raccoon, birds such as hens, geese, turkeys and ducks and fish such as fresh- and salt-water fish such as trout, carp and eels. Particularly preferred are domestic animals, such as dogs or cats.

In general, “parasiticidally effective amount” means the amount of active ingredient needed to achieve an observable effect on growth, including the effects of necrosis, death, retardation, prevention, and removal, destruction, or otherwise diminishing the occurrence and activity of the target organism. The parasiticidally effective amount can vary for the various compounds/compositions used in the invention. A parasiticidally effective amount of the compositions will also vary according to the prevailing conditions such as desired parasiticidal effect and duration, target species, mode of application, and the like.

Generally, it is favorable to apply the compounds of the present invention in total amounts of 0.5 mg/kg to 100 mg/kg per day, preferably 1 mg/kg to 50 mg/kg per day.

For oral administration to warm-blooded animals, the formula I compounds may be formulated as animal feeds, animal feed premixes, animal feed concentrates, pills, solutions, pastes, suspensions, drenches, gels, tablets, boluses and capsules. In addition, the formula I compounds may be administered to the animals in their drinking water. For oral administration, the dosage form chosen should provide the animal with 0.01 mg/kg to 100 mg/kg of animal body weight per day of the formula I compound, preferably with 0.5 mg/kg to 100 mg/kg of animal body weight per day.

Alternatively, the formula I compounds may be administered to animals parenterally, for example, by intraruminal, intramuscular, intravenous or subcutaneous injection. The formula I compounds may be dispersed or dissolved in a physiologically acceptable carrier for subcutaneous injection. Alternatively, the formula I compounds may be formulated into an implant for subcutaneous administration. In addition, the formula I compound may be transdermally administered to animals. For parenteral administration, the dosage form chosen should provide the animal with 0.01 mg/kg to 100 mg/kg of animal body weight per day of the formula I compound.

The formula I compounds may also be applied topically to the animals in the form of dips, dusts, powders, collars, medallions, sprays, shampoos, spot-on and pour-on formulations and in ointments or oil-in-water or water-in-oil emulsions. For topical application, dips and sprays usually contain 0.5 ppm to 5,000 ppm and preferably 1 ppm to 3,000 ppm of the formula I compound. In addition, the formula I compounds may be formulated as ear tags for animals, particularly quadrupeds such as cattle and sheep.

Suitable preparations are:

Solutions such as oral solutions, concentrates for oral administration after dilution, solutions for use on the skin or in body cavities, pouring-on formulations, gels;

Emulsions and suspensions for oral or dermal administration; semi-solid preparations;

Formulations in which the active compound is processed in an ointment base or in an oil-in-water or water-in-oil emulsion base;

Solid preparations such as powders, premixes or concentrates, granules, pellets, tablets, boluses, capsules; aerosols and inhalants, and active compound-containing shaped articles.

Compositions suitable for injection are prepared by dissolving the active ingredient in a suitable solvent and optionally adding further auxiliaries such as acids, bases, buffer salts, preservatives, and solubilizers. Suitable auxiliaries for injection solutions are known in the art. The solutions are filtered and filled sterile.

Oral solutions are administered directly. Concentrates are administered orally after prior dilution to the use concentration. Oral solutions and concentrates are prepared according to the state of the art and as described above for injection solutions, sterile procedures not being necessary.

Solutions for use on the skin are trickled on, spread on, rubbed in, sprinkled on or sprayed on. Solutions for use on the skin are prepared according to the state of the art and according to what is described above for injection solutions, sterile procedures not being necessary.

Gels are applied to or spread on the skin or introduced into body cavities. Gels are prepared by treating solutions which have been prepared as described in the case of the injection solutions with sufficient thickener that a clear material having an ointment-like consistency results. Suitable thickeners are known in the art.

Pour-on formulations are poured or sprayed onto limited areas of the skin, the active compound penetrating the skin and acting systemically. Pour-on formulations are prepared by dissolving, suspending or emulsifying the active compound in suitable skin-compatible solvents or solvent mixtures. If appropriate, other auxiliaries such as colorants, bioabsorption-promoting substances, antioxidants, light stabilizers, adhesives are added. Suitable such auxiliaries are known in the art.

Emulsions can be administered orally, dermally or as injections. Emulsions are either of the water-in-oil type or of the oil-in-water type. They are prepared by dissolving the active compound either in the hydrophobic or in the hydrophilic phase and homogenizing this with the solvent of the other phase with the aid of suitable emulsifiers and, if appropriate, other auxiliaries such as colorants, absorption-promoting substances, preservatives, antioxidants, light stabilizers, viscosity-enhancing substances. Suitable hydrophobic phases (oils), suitable hydrophilic phases, suitable emulsifiers, and suitable further auxiliaries for emulsions are known in the art.

Suspensions can be administered orally or topically/dermally. They are prepared by suspending the active compound in a suspending agent, if appropriate with addition of other auxiliaries such as wetting agents, colorants, bioabsorption-promoting substances, preservatives, antioxidants, light stabilizers. Suitable suspending agents, and suitable other auxiliaries for suspensions including wetting agents are known in the art.

Semi-solid preparations can be administered orally or topically/dermally. They differ from the suspensions and emulsions described above only by their higher viscosity.

For the production of solid preparations, the active compound is mixed with suitable excipients, if appropriate with addition of auxiliaries, and brought into the desired form. Suitable auxiliaries for this purpose are known in the art.

The compositions which can be used in the invention can comprise generally from about 0.001 to 95% of the compound of the present invention.

Ready-to-use preparations contain the compounds acting against parasites, preferably ectoparasites, in concentrations of 10 ppm to 80 percent by weight, preferably from 0.1 to 65 per cent by weight, more preferably from 1 to 50 percent by weight, most preferably from 5 to 40 percent by weight.

Preparations which are diluted before use contain the compounds acting against ectoparasites in concentrations of 0.5 to 90 percent by weight, preferably of 1 to 50 percent by weight.

Furthermore, the preparations comprise the compounds of formula I against endoparasites in concentrations of 10 ppm to 2 percent by weight, preferably of 0.05 to 0.9 percent by weight, very particularly preferably of 0.005 to 0.25 percent by weight.

Topical application may be conducted with compound-containing shaped articles such as collars, medallions, ear tags, bands for fixing at body parts, and adhesive strips and foils.

Generally it is favorable to apply solid formulations which release compounds of the present invention in total amounts of 10 ring/kg to 300 mg/kg, preferably 20 mg/kg to 200 mg/kg, most preferably 25 mg/kg to 160 mg/kg body weight of the treated animal in the course of three weeks.

### EXAMPLES

### A. Preparation examples

With appropriate modification of the starting materials, the procedures given in the synthesis description were used to obtain further compounds I. The compounds obtained in this manner are listed in the table that follows, together with physical data.

The products shown below were characterized by melting point determination, by NMR spectroscopy or by the masses ([m/z]) or retention time (RT; [min.]) determined by HPLC-MS or HPLC spectrometry.

HPLC-MS=high performance liquid chromatography-coupled mass spectrometry;

HPLC method A: HPLC Phenomenex Kinetex 1.7μm XB-C18 100A, 50×2.1 mm″, Mobile Phase: A: water+0.1% TFA; B:CAN; Temperature: 60° C.; Gradient:5% B to 100% B in 1.50min; 100% B 0.25min; Flow: 0.8 ml/min to 1.0 ml/min in 1.51 min; MS method: ESI positive; Mass range (m/z): 100-700″.

HPLC method B: HPLC method: Phenomenex Kinetex 1.7 μm XB-C18 100A; 50×2.1 mm; mobile phase: A: water+0.1% trifluoroacetic acid (TFA); B: acetonitrile; gradient: 5-100% B in 1.50 minutes; 100% B 0.25 min; flow: 0.8-1.0ml/min in 1.51 minutes at 60° C. MS: ESI positive, m/z 100-700.

Starting material tert-butyl 6-[(E/Z)-3-(3,5-dichloro-4-fluoro-phenyl)-4,4,4-trifluoro-but-2-enoyl]spiro[1H-isobenzofuran-3,3′-azetidine]-1′-carboxylate was prepared according to WO 2016/115315. Tert-butyl 6-RE/Z)-3-(3,5-dichlorophenyl)-4,4,4-trifluoro-but-2-enoyl]spiro[1H-isobenzofuran-3,3′-azetidine]-1′-carboxylate and tert-butyl 6-[(E/Z)-3-(3-chloro-4-fluoro-phenyl)-4,4,4-trifluoro-but-2-enoyl]spiro[1H-isobenzofuran-3,3′-azetidine]-1′-carboxylate were prepared analogously.

### Example 1

Preparation of 1-[6-[4-(3,5-dichloro-4-fluoro-phenyl)-4-(trifluoromethyl)cyclopenten-1-yl]spiro[1H-isobenzofuran-3,3′-azetidine]-1′-yl]propan-1-one [No. I-1-1]

Step 1: tert-butyl 6-(E/Z)-1-allyl-3-(3,5-dichloro-4-fluoro-phenyl)-4,4,4-trifluoro-1-hydroxy-but-2-enyl]spiro[1H-isobenzofuran-3,3′-azetidine]-1′-carboxylate

To a solution of tert-butyl 6-[(E/Z)-3-(3,5-dichloro-4-fluoro-phenyl)-4,4,4-trifluoro-but-2-enoyl]spiro[1H-isobenzofuran-3,3′-azetidine]-t-carboxylate (117.3 g, 214.7 mmol) in THF (1 L) was added a solution of CH₃MgBr (1.05 M in THF, 322 mL, 322 mmol, 1.5 equiv.) at 0° C. After 1.5 h at this temperature, saturated aqueous NH₄Cl-solution was added and the mixture was stirred into MTBE (1 L). The organic layer was separated, washed with water and dried over Na₂SO₄. After removal of the volatiles in vacuum, the title compound (125.4 g, 99%) was obtained and used as such in the next step.

Step 2: tert-butyl 6-[3-(3,5-dichloro-4-fluoro-phenyl)-3-(trifluoromethyl)hex-5-enoyl]spiro[1H-isobenzofuran-3,3′-azetidine]-1′-carboxylate

To a stirred solution of KHMDS (1175 mL of a 0.5 M solution in toluene, 588 mmol, 3 equiv.) and 18-crown-6 (155.4 g, 588 mmol, 3 equiv.) in THF (500 mL) was added tert-butyl 6-[(E/Z)-1-allyl-3-(3,5-dichloro-4-fluoro-phenyl)-4,4,4-trifluoro-1-hydroxy-but-2-enyl]spiro[1H-isobenzofuran-3,3′-azetidine]-1′-carboxylate (115.3 g, 196 mmol) in THF (500 mL) at −25° C. to −20° C. After 1 h at −20° C., the mixture was allowed to warm to 0° C. and kept for 15 min, before water was added. The mixture was stirred into MTBE (1.5 L) and the layers were separated. The aqueous layer was extracted with MTBE and combined organic layers were washed with water, and saturated aqueous NaCl solution. The resulting solution was dried over Na₂SO₄ and evaporated. Column chromatography over silica gel yielded the title compound (78.5 g, 68%).

¹H NMR: (500 MHz, CDCl₃): δ 7.93 (d, 1H), 7.74 (s, 1H), 7.57 (d, 1H), 7.33 (m, 2H), 5.84 (m, 1H), 5.16 (s, 2H), 5.07 (m, 2H), 4.33 (d, 2H), 4.13 (d, 2H), 3.81 (d, 1H), 3.70 (d, 1H), 3.17 (m, 2H), 1.50 (s, 9H) ppm.

Step 3: tert-butyl 6-[3-(3,5-dichloro-4-fluoro-phenyl)-1-methylene-3-(trifluoromethyl)hex-5-enyl]spiro[1H-isobenzofuran-3,3′-azetidine]-1′-carboxylate

To a suspension of methyl triphenylphosphonium bromide (59.495 g, 166.55 mmol, 1.25 equiv.) in toluene (800 mL) was added KHMDS (306 mL of a 0.5 M solution on toluene, 153 mmol, 1.15 equiv.) at room temperature. After 1 h, a solution of tert-butyl 6-[3-(3,5-dichloro-4-fluoro-phenyl)-3-(trifluoromethyl)hex-5-enoyl]spiro[1H-isobenzofuran-3,3′-azetidine]-1′-carboxylate (78.40 g, 133.2 mmol) in toluene (400 mL) was added at 0° C. The reaction mixture was allowed to warm to room temperature over night and water was added. The resulting emulsion was stirred into ethyl acetate (1 L) and the layers were separated. The organic layer was washed with water (3×) and dried over Na₂SO₄. All volatiles were removed in vacuum and the residue was purified via column chromatography on silica gel to obtain the title compound (77.6 g, 99%).

¹H NMR: (400 MHz, CDCl₃): δ]7.25 (d, 1H), 7.18 (d, 2H), 6.98 (d, 1H), 6.80 (s, 1H), 5.62 (m, 1H), 5.30-5.09 (m, 4H), 5.01 (m, 2H), 4.29 (m, 2H), 4.11 (m, 2H), 3.23 (d, 1H), 2.98 (d, 1H), 2.95-2.84 (m, 1H), 2.72-2.64 (m, 1H), 1.50 (s, 9H) ppm.

Step 4: tert-butyl 6-[4-(3,5-dichloro-4-fluoro-phenyl)-4-(trifluoromethyl)cyclopenten-1-yl]spiro[1H-isobenzofuran-3,3′-azetidine]-1′-carboxylate

To a solution of tert-butyl 6-[3-(3,5-dichloro-4-fluoro-phenyl)-1-methylene-3-(trifluoromethyl)hex-5-enyl]spiro[1H-isobenzofuran-3,3′-azetidine]-1′-carboxylate (77.5 g, 132 mmol) in CH₂Cl₂ (2 L) was added (1,3-bis(2,4,6-trimethylphenyl)-2-imidazolidinylidene)dichloro(phenylmethylene)-(tricyclohexylphosphine)ruthenium (Grubbs Catalyst 2nd generation, 3.366 g, 3.965 mmol, 3 mol-%) at room temperature and the mixture was stirred over night. Filtration over a short plug of silica gel yielded the title compound (72.7 g, 99%).

¹H NMR: (500 MHz, CDCl₃): δ 7.46 (m, 2H), 7.40 (m, 2H), 7.27 (s, 1H), 6.18 (s, 1H), 5.12 (s, 2H), 4.32 (d, 1H), 4.14 (d, 1H), 4.33-4.24 (m, 2H), 3.55 (d, 1H), 3.38 (d, 1H), 3.28 (d, 1H), 3.10 (d, 1H), 1.50 (s, 9H) ppm.

Step 5: 6-[4-(3,5-dichloro-4-fluoro-phenyl)-4-(trifluoromethyl)cyclopenten-1-yl]spiro[1H-iso-benzofuran-3,3′-azetidine]-trifluoroacetate

To a solution of tert-butyl 6-[4-(3,5-dichloro-4-fluoro-phenyl)-4-(trifluoromethyl)cyclopenten-1-yl]spiro[1H-isobenzofuran-3,3′-azetidine]-1′-carboxylate in CH₂Cl₂ (1 L) was added trifluoroacetic acid (500 mL) at 0° C. After 45 min, all volatiles were removed in vacuum at 20° C. and the crude title compound (71.2 g, 100%) was used in the next step without further purification.

Step 6: 1-[6-[4-(3,5-dichloro-4-fluoro-phenyl)-4-(trifluoromethyl)cyclopenten-1-yl]spiro[1H-iso-benzofuran-3,3′-azetidine]-1′-yl]propan-1-one [No. I-1-1]

To a solution of 6-[4-(3,5-dichloro-4-fluoro-phenyl)-4-(trifluoromethyl)cyclopenten-1-yl]spiro[1H-isobenzofuran-3,3′-azetidine]-trifluoroacetate (712 mg 1.24 mmol), DMAP (15 mg, 0.12 mmol, 10 mol-%), Hünig base (965 mL, 7.46 mmol, 6 equiv.) in THF 15 mL was added propionyl chloride (138 mg, 1.49 mmol, 1.20 equiv.) in THF (3 mL) at 0° C. The mixture was allowed to warm to room temperature and stirred over night. Water was added and the mixture was extracted with ethyl acetate three times. Combined organic layers were washed with brine and dried over Na₂SO₄. The solvents were removed in vacuum and the residue was purified through flash chromatography to obtain the title compound (508 mg, 79%).

¹H NMR: (400 MHz, CDCl₃): δ7.46 (d, 1H), 7.40 (m, 3H), 7.29 (s, 1H), 6.18 (s, 1H), 5.14 (s, 2H), 4.47 (d, 1H), 4.37 (d, 1H), 4.33-4.24 (m, 2H), 3.55 (d, 1H), 3.39 (d, 1H), 3.27 (d, 1H), 3.11 (d, 1H), 2.21 (q, 2H), 1.20 (t, 3H) ppm.

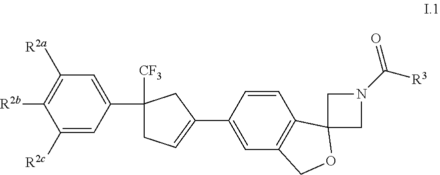

TABLE C.1

| | | | HPLC | M + H | HPLC |
|---|---|---|---|---|---|
| No. | R^{2a}, R^{2b}, R^{2c} | R³ | Rt [min] | [m/z] | Method |
| 1-1-1 | Cl, F, Cl | C₂H₅ | 1.437 | 514.1 | A |
| 1-1-2 | Cl, F, Cl | CH₃ | 1.379 | 500.1 | A |
| 1-1-3 | Cl, F, Cl | CH(CH₃)₂ | 1.470 | 528.0 | A |
| 1-1-4 | Cl, F, Cl | CH₂CH(CH₃)₂ | 1.507 | 542.1 | A |
| 1-1-5 | Cl, F, Cl | 3-pyridyl | 1.306 | 563.1 | A |
| 1-1-6 | Cl, F, Cl | CH₂CF₃ | 1.454 | 568.1 | A |
| 1-1-7 | Cl, F, Cl | c-C₄H₇ | 1.501 | 540.1 | A |
| 1-1-8 | Cl, F, Cl | c-C₃H₅ | 1.477 | 526.0 | A |
| 1-1-9 | Cl, F, Cl | CH₂S(O)₂CH₃ | 1.371 | 578.0 | A |
| 1-1-10 | Cl, F, Cl | C≡CH | 1.460 | 510.0 | A |
| 1-1-11 | Cl, F, Cl | CH₂-1,3- | 1.388 | 572.1 | A |
| | | dioxolan-2-yl | | | |
| 1-1-12 | Cl, H, Cl | c-C₃H₅ | 1.450 | 508.1 | A |
| 1-1-13 | Cl, H, Cl | C₂H₅ | 1.433 | 496.1 | A |
| 1-1-14 | Cl, H, Cl | CH₂CF₃ | 1.453 | 550.1 | A |
| 1-1-15 | Cl, F, H | C₂H₅ | 1.382 | 480.1 | A |
| 1-1-16 | Cl, F, H | c-C₃H₅ | 1.400 | 492.1 | A |
| 1-1-17 | Cl, H, Cl | CH₂CH₂CH₃ | 1.501 | 510.1 | A |
| 1-1-18 | Cl, F, H | CH₂CF₃ | 1.382 | 534.1 | A |
| 1-1-19 | Cl, F, H | CH₂CH₂CH₃ | 1.399 | 494.1 | A |

### Example 2

Synthesis of 1-[6-[2-(3,5-dichloro-4-fluoro-phenyl)-2-(trifluoromethyl)-3H-thiophen-4-yl]spiro[1H-isobenzofuran-3,3′-azetidine]-1′-yl]ethanone (Compound I-2-2)

Step 1: tert-butyl 6-[5-(3,5-dichloro-4-fluoro-phenyl)-3-hydroxy-2-methoxycarbonyl-5-(trifluoromethyl)tetrahydrothiophen-3-yl]spiro[1H-isobenzofuran-3,3′-azetidine]-1′-carboxylate To a stirred solution of LiBr (1.749 g, 0.020 mol, 2.2 equiv.) and Hünig base (0.845 mL, 591 mg, 0.5 equiv.) in THF (100 mL) was added ethyl thioglycolate (0.906 mL, 1.069 g, 1.1 equiv.) and cooled to 0° C. To this suspension, a solution of tert-butyl 6-[(E/Z)-3-(3,5-dichloro-4-fluoro-phenyl)-4,4,4-trifluoro-but-2-enoyl]spiro[1H-isobenzofuran-3,3′-azetidine]-1′-carboxylate (5.00 g, 0.009 mol) in THF (50 mL) was added and left for 2 h at this temperature. Water was added and the mixture was extracted with ethyl acetate three times. The combined organic layers were dried over Na₂SO₄ and concentrated under reduced pressure to obtain the crude product. Purification via silica gel chromatography yielded the title compound (5.80 g, 97%). ¹H NMR: (300 MHz, CDCl₃): δ7.62 (d, 2H), 7.50 (s, 2H), 7.39 (s, 1H), 5.32-4.68 (m, 4H), 4.32 (d, 2H), 4.13 (d, 2H), 3.72 (s, 3H), 2.92 (m, 1H) 1.50 (s, 9H) ppm.

Step 2: 3-(1′-tert-butoxycarbonylspiro[3H-isobenzofuran-1,3′-azetidine]-5-yl)-5-(3,5-dichloro-4-fluoro-phenyl)-3-hydroxy-5- (trifluoromethyl)tetrahydrothiophene-2-carboxylic acid To a solution of tert-butyl 6-[5-(3,5-dichloro-4-fluoro-phenyl)-3-hydroxy-2-methoxycarbonyl-5-(trifluoromethyl)tetrahydrothiophen-3-yl]spiro[1H-isobenzofuran-3,3′-azetidine]-1′-carboxylate (5.80 g, 8.89 mmol) in THF (50 mL) and water (12 mL) was added LiOH (746 mg, 0.018 mol, 2 equiv.) and the mixture was stirred at room temperature for 3 h. Water was added and acidified with 1 N HCl. The resulting suspension was extracted with ethyl acetate three times. Combined organic layers were dried over Na₂SO₄ and concentrated under reduced pressure to get the crude product (5.60 g, 99%) which was used in the next step without further purification.

Step 3: tert-butyl 6-[3-(3,5-dichloro-4-fluoro-phenyl)-7-oxo-3-(trifluoromethyl)-6-oxa-2-thiabicyclo[3.2.0]heptan-5-yl]spiro[1H-isobenzofuran-3,3′-azetidine]-1′-carboxylate

To a solution of 3-(1′-tert-butoxycarbonylspiro[3H-isobenzofuran-1,3′-azetidine]-5-yl)-5-(3,5-dichloro-4-fluoro-phenyl)-3-hydroxy-5-(trifluoromethyl)tetrahydrothiophene-2-carboxylic acid (5.60 g, 8.771 mmol) in pyridine (50 mL) was added methane sulfonyl chloride (1.02 mL, 1.51 g, 13.2 mmol, 1.5 equiv.) at 0° C. The mixture was allowed to warm to room temperature and stirred for 3 h, before it was poured into water. The resulting mixture was acidified with 1 N HCl and extracted with ethyl acetate three times. Combined organic layers were dried over Na₂SO₄ and concentrated under reduced pressure to get the crude title compound (5.3 g, 97%) which was used in the next step without further purification.

Step 4: tert-butyl 6-[2-(3,5-dichloro-4-fluoro-phenyl)-2-(trifluoromethyl)-3H-thiophen-4-yl]spiro[1H-isobenzofuran-3,3′-azetidine]-1′-carboxylate

A solution of tert-butyl 6-[3-(3,5-dichloro-4-fluoro-phenyl)-7-oxo-3-(trifluoromethyl)-6-oxa-2-thiabicyclo[3.2.0]heptan-5-yl]spiro[1H-isobenzofuran-3,3′-azetidine]-1′-carboxylate (5.4 g, 8.7 mmol) in DMF (50 mL) was heated at 100° C. for 3 h. After cooling, water was added and the mixture was extracted with ethyl acetate three times. Combined organic layers were dried over Na₂SO₄ and concentrated under reduced pressure to get the crude product. Purification via silica gel chromatography yielded the title compound (4.00 g, 80%).

¹H NMR: (300 MHz, CDCl₃): δ7.52 (d, 2H), 7.47-7.37 (m, 2H), 7.20 (s, 1H), 6.54 (s, 1H), 5.13 (s, 2H), 4.32 (d, 1H), 4.13 (d, 1H), 3.88 (m, 1H), 3.65 (d, 1H), 1.50 (s, 9H) ppm.

Step 5: 6-[2-(3,5-dichloro-4-fluoro-phenyl)-2-(trifluoromethyl)-3H-thiophen-4-yl]spiro[1H-iso-benzofuran-3,3′-azetidine] hydrochloride

To a solution of tert-butyl 6-[2-(3,5-dichloro-4-fluoro-phenyl)-2-(trifluoromethyl)-3H-thiophen-4-yl]spiro[1H-isobenzofuran-3,3′-azetidine]-1′-carboxylate (3.70 g, 6.42 mmol) in CH₂Cl₂ (8 mL) was added a solution of HCl (8 mL of a 4 M solution in 1,4-dioxane, 32 mmol, 5 equiv.) at 0° C. and the mixture was allowed to warm to room temperature. All volatiles were removed in vacuum to obtain the title compound (3.00 g, 91%) which was used in the next step without purification.

¹H NMR: (300 MHz, CDCl₃): δ7.67 (m, 1H), 7.45-7.31 (m, 2H), 7.25 (m, 1H), 7.10 (m, 1H), 6.42 (m, 1H), 5.23 (s, 2H), 4.12-4.02 (m, 2H), 3.83-3.42 (m, 6H) ppm.

Step 6: 1-[6-[2-(3,5-dichloro-4-fluoro-phenyl)-2-(trifluoromethyl)-3H-thiophen-4-yl]spiro[1H-iso-benzofuran-3,3′-azetidine]-1′-yl]ethanone

Acetyl chloride (115 mg, 1.46 mmol, 2.5 equiv.) in CH₂Cl₂ (5 mL) was added to a solution of 6-[2-(3,5-dichloro-4-fluoro-phenyl)-2-(trifluoromethyl)-3H-th iophen-4-yl]spiro[1H-isobenzofuran-3,3′-azetidine] hydrochloride (300 mg, 585 mmol) and triethyl amine (118 mg, 1.17 mmol, 2.0 equiv.) in CH₂Cl₂ (10 mL) at 0° C., and the mixture was allowed to warm to 20-25° C. After 2 h, water was added and the mixture was extracted with ethyl acetate. Combined organic layers were dried and concentrated under reduced pressure to get the crude product. Purification via silica gel chromatography yielded the title compound (135 mg, 41%).

¹H NMR: (300 MHz, CDCl₃): δ7.50 (d, J=6.0 Hz, 2H), 7.45-7.33 (m, 2H), 7.20 (s, 1H), 6.53 (d, J=1.9 Hz, 1H), 5.13 (s, 2H), 4.54-4.19 (m, 4H), 3.86 (d, J=16.4, 1H), 3.64 (d, J=16.2, 1H), 1.64 (s, 3H) ppm.

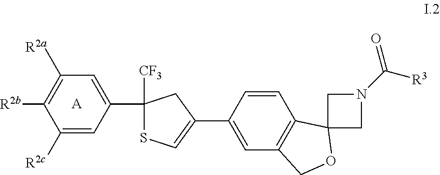

TABLE C.2

| | | | HPLC | M + H | HPLC |
|---|---|---|---|---|---|
| No. | R^{2a}, R^{2b}, R^{2c} | R³ | Rt [min] | [m/z] | Method |
| 1-2-1 | Cl, F, Cl | OC(CH₃)₃ | 2.680 | 575.0 | B |
| 1-2-2 | Cl, F, Cl | C₂H₅ | 2.390 | 531.0 | B |
| 1-2-3 | Cl, F, Cl | CH₃ | 2.320 | 518.0 | B |
| 1-2-4 | Cl, F, Cl | CH₂CH₂CH₃ | 2.440 | 546.0 | B |
| 1-2-5 | Cl, F, Cl | c-C₃H₅ | 2.390 | 544.0 | B |
| 1-2-6 | Cl, F, Cl | CH₂CF₃ | 2.360 | 585.0 | B |
| 1-2-7 | Cl, F, Cl | CH₂S(O)₂CH₃ | 2.240 | 595.0 | B |

### II. Evaluation of Pesticidal Activity

The activity of the compounds of formula I of the present invention can be demonstrated and evaluated by the following biological test.

### B.1 Diamond Back Moth (Plutella xylostella)

The active compound was dissolved at the desired concentration in a mixture of 1:1 (vol:vol) distilled water:aceteone. Surfactant (Kinetic HV) was added at a rate of 0.01% (vol/vol).The test solution was prepared at the day of use.

Leaves of cabbage were dipped in test solution and air-dried. Treated leaves were placed in petri dishes lined with moist filter paper and inoculated with ten 3^{rd} instar larvae. Mortality was recorded 72 hours after treatment. Feeding damages were also recorded using a scale of 0-100%.

In this test, the compounds I-1-1, I-2-1, I-1-2, I-2-2, I-1-3, I-2-3, I-1-4, I-2-4, I-1-5, I-2-5, I-2-6, I-2-7 at 300 ppm, respectively, showed a mortality of at least 75% in comparison with untreated controls.

### B.2 Green Peach Aphid (Myzus persicae)

For evaluating control of green peach aphid (Myzus persicae) through systemic means the test unit consisted of 96-well-microtiter plates containing liquid artificial diet under an artificial membrane.

The compounds were formulated using a solution containing 75% v/v water and 25% v/v DMSO. Different concentrations of formulated compounds were pipetted into the aphid diet, using a custom built pipetter, at two replications.

After application, 5-8 adult aphids were placed on the artificial membrane inside the microtiter plate wells. The aphids were then allowed to suck on the treated aphid diet and incubated at about 23±1 ° C. and about 50±5% relative humidity for 3 days. Aphid mortality and fecundity was then visually assessed.

In this test, the compounds I-1-1, I-1-2, I-2-2, I-1-3, I-2-3, I-1-4, I-2-4, I-1-5, I-2-5, I-2-6, I-2-7, at 2500 ppm, respectively, showed a mortality of at least 75% in comparison with untreated controls.

### B.3 Vetch Aphid (Megoura viciae)

For evaluating control of vetch aphid (Megoura viciae) through contact or systemic means the test unit consisted of 24-well-microtiter plates containing broad bean leaf disks.

The compounds were formulated using a solution containing 75% v/v water and 25% v/v DMSO. Different concentrations of formulated compounds were sprayed onto the leaf disks at 2.5 μl, using a custom built micro atomizer, at two replications.

After application, the leaf disks were air-dried and 5- 8 adult aphids placed on the leaf disks inside the microtiter plate wells. The aphids were then allowed to suck on the treated leaf disks and incubated at about 23±1° C. and about 50±5% relative humidity for 5 days. Aphid mortality and fecundity was then visually assessed.

In this test, the compounds I-1-1, I-2-2, I-1-3, I-2-3, I-1-4, I-2-4, I-1-5, I-2-5, I-2-6, I-2-7 at 2500 ppm, respectively, showed a mortality of at least 75% in comparison with untreated controls.

### B.4 Tobacco Budworm (Heliothis virescens)

For evaluating control of tobacco budworm (Heliothis virescens) the test unit consisted of 96-well-microtiter plates containing an insect diet and 15-25 H. virescens eggs.

The compounds were formulated using a solution containing 75% v/v water and 25% v/v DMSO. Different concentrations of formulated compounds were sprayed onto the insect diet at 10 μl, using a custom built micro atomizer, at two replications.

After application, microtiter plates were incubated at about 28+1° C. and about 80+5% relative humidity for 5 days. Egg and larval mortality was then visually assessed.

In this test, the compounds I-1-1, I-2-1, I-2-2, I-1-3, I-2-3, I-1-4, I-2-4, I-1-5, I-2-5, I-2-6, I-2-7, at 2500 ppm, respectively, showed a mortality of at least 75% in comparison with untreated controls.

### B.5 Boll Weevil (Anthonomus grandis)

For evaluating control of boll weevil (Anthonomus grandis) the test unit consisted of 96-well-microtiter plates containing an insect diet and 5-10 A. grandis eggs.

The compounds were formulated using a solution containing 75% v/v water and 25% v/v DMSO. Different concentrations of formulated compounds were sprayed onto the insect diet at 5 μl, using a custom built micro atomizer, at two replications.

After application, microtiter plates were incubated at about 25±1° C. and about 75±5% relative humidity for 5 days. Egg and larval mortality was then visually assessed.

In this test, the compounds I-1-1, I-2-1, I-1-2, I-2-2, I-1-3, I-2-3, I-1-4, I-2-4, I-1-5, I-2-5, I-2-6, I-2-7 at 2500 ppm, respectively, showed a mortality of at least 75% in comparison with untreated controls.

### B.6 Mediterranean Fruitfly (Ceratitis capitata)

For evaluating control of Mediterranean fruitfly (Ceratitis capitata) the test unit consisted of microtiter plates containing an insect diet and 50-80 C. capitata eggs.

The compounds were formulated using a solution containing 75% v/v water and 25% v/v DMSO. Different concentrations of formulated compounds were sprayed onto the insect diet at 5 μl, using a custom built micro atomizer, at two replications.

After application, microtiter plates were incubated at about 28±1° C. and about 80±5% relative humidity for 5 days. Egg and larval mortality was then visually assessed.

In this test, the compounds I-1-1, I-2-1, I-1-3, I-1-4, I-2-4, I-1-5, I-2-5, I-2-6, I-2-7 at 2500 ppm, respectively, showed a mortality of at least 75% in comparison with untreated controls.

### B.7 Orchid Thrips (dichromothrips corbetti)

Dichromothrips corbetti adults used for bioassay were obtained from a colony maintained continuously under laboratory conditions. For testing purposes, the test compound is diluted in a 1:1 mixture of acetone:water (vol:vol), plus Kinetic HV ata rate of 0.01% v/v.

Thrips potency of each compound was evaluated by using a floral-immersion technique. All petals of individual, intact orchid flowers were dipped into treatment solution and allowed to dry in Petri dishes. Treated petals were placed into individual re-sealable plastic along with about 20 adult thrips. All test arenas were held under continuous light and a temperature of about 28° C. for duration of the assay. After 3 days, the numbers of live thrips were counted on each petal. The percent mortality was recorded 72 hours after treatment.

In this test, the compounds I-1-1, I-2-1, I-1-2, I-2-2, I-1-3, I-2-3, I-1-4, I-2-4, I-1-5, I-2-5, I-2-6, I-2-7 at 300 ppm, respectively, showed a mortality of at least 75% in comparison with untreated controls.

### B.8 Rice green leafhopper (Nephotettix virescens)

Rice seedlings were cleaned and washed 24 hours before spraying. The active compounds were formulated in 1:1 acetone:water (vol:vol), and 0.01% vol/vol surfactant (Kinetic HV) was added. Potted rice seedlings were sprayed with 5-6 ml test solution, air dried, covered with Mylar cages cages and inoculated with 10 adults. Treated rice plants were kept at about 28-29° C. and relative humidity of about 50-60%. Percent mortality was recorded after 72 hours.

In this test, the compounds I-1-1, I-1-2, I-2-2, I-1-3, I-2-3, I-2-4, I-2-5, I-2-6 at 300 ppm, respectively, showed a mortality of at least 75% in comparison with untreated controls.

### B.9 Red Spider Mite (Tetranychus kanzawai)

The active compound was dissolved at the desired concentration in a mixture of 1:1 (vol:vol) distilled water:acetone. Add surfactant (Kinetic HV) was added at a rate of 0.01% (vol/vol).The test solution was prepared at the day of use.

Potted cowpea beans of 4-5 days of age were cleaned with tap water and sprayed with 1-2 ml of the test solution using air driven hand atomizer. The treated plants were allowed to air dry and afterwards inoculated with 30 or more mites by clipping a cassava leaf section from rearing population. Treated plants were placed inside a holding room at about 25-27° C. and about 50-60% relative humidity. Percent mortality was assessed 72 hours after treatment.

In this test, the compounds I-1-1, I-1-2, I-2-2, I-1-3, I-2-3, I-1-4, I-2-4, I-2-5, I-2-6 at 300 ppm, respectively, showed a mortality of at least 75% in comparison with untreated controls.

### B.10 Southern Armyworm (Spodoptera eridania)

The active compounds were formulated in cyclohexanone as a 10,000 ppm solution supplied in tubes. The tubes were inserted into an automated electrostatic sprayer equipped with an atomizing nozzle and they served as stock solutions for which lower dilutions were made in 50% acetone:50% water (v/v). A nonionic surfactant (Kinetic®) was included in the solution at a volume of 0.01% (v/v).

Lima bean plants (variety Sieva) were grown 2 plants to a pot and selected for treatment at the 1st true leaf stage. Test solutions were sprayed onto the foliage by an automated electrostatic plant sprayer equipped with an atomizing spray nozzle. The plants were dried in the sprayer fume hood and then removed from the sprayer. Each pot was placed into perforated plastic bags with a zip closure. About 10 to 11 armyworm larvae were placed into the bag and the bags zipped closed. Test plants were maintained in a growth room at about 25° C. and about 20-40% relative humidity for 4 days, avoiding direct exposure to fluorescent light (24 hour photoperiod) to prevent trapping of heat inside the bags. Mortality and reduced feeding were assessed 4 days after treatment, compared to untreated control plants.

In this test, the compounds I-1-1, I-2-2, I-2-3, I-2-4, I-2-5, I-2-6 at 10 ppm, respectively, showed a mortality of at least 75% in comparison with untreated controls.

### B.11 Green Soldier Stink Bug (Nezara viridula)

The active compound was dissolved at the desired concentration in a mixture of 1:1 (vol:vol) distilled water:acetone. Surfactant (Kinetic HV) was added at a rate of 0.01% (vol/vol). The test solution was prepared at the day of use.Soybean pods were placed in glass Petri dishes lined with moist filter paper and inoculated with ten late 3rd instar N. vindula. Using a hand atomizer, approximately 2 ml solution is sprayed into each Petri dish. Assay arenas were kept at about 25° C. Percent mortality was recorded after 5 days.

In this test, the compounds 1-1-1, 1-1-2, 1-2-2, 1-1-3, 1-2-3,1-2-4, 1-2-5, 1-2-6, 1-2-7 at 300 ppm, respectively, showed a mortality of at least 75% in comparison with untreated controls.

### B.12 Neotropical Brown Stink Bug (Euschistus heros)

The active compound was dissolved at the desired concentration in a mixture of 1:1 (vol:vol) distilled water:acetone. Surfactant (Kinetic HV) was added at a rate of 0.01% (vol/vol). The test solution was prepared at the day of use.

Soybean pods were placed in microwavable plastic cups and inoculated with ten adult stage E. heros. Using a hand atomizer, approximately 1 ml solution is sprayed into each cup, insects and food present. A water source was provided (cotton wick with water). Each treatment was replicated 2-fold. Assay arenas were kept at about 25° C. Percent mortality was recorded after 5 days.

In this test, the compounds I-2-3 and I-2-4 at 100 ppm, respectively, showed a mortality of at least 70% in comparison with untreated controls.

### B.13 Brown Marmorated Stink Bug (Halyomorpha halys)

The active compound was dissolved at the desired concentration in a mixture of 1:1 (vol:vol) distilled water:acetone. Surfactant (Kinetic HV) was added at a rate of 0.01% (vol/vol). The test solution was prepared at the day of use.

Row peanuts and soybean seeds were placed into microwavable plastic cups and inoculated with five adult stage H. halys. Using a hand atomizer, approximately 1 ml solution is sprayed into each cup, insects and food present. A water source was provided (cotton wick with water). Each treatment is replicated 4-fold. Assay arenas are kept at about 25° C. Percent mortality was recorded after 5 days.

In this test, the compounds I-1-1, I -2-2, I-2-3, I-2-5, I-2-6 at 100 ppm, respectively, showed a mortality of at least 75% in comparison with untreated controls.

1-15. (canceled)

16. A spiro compound of formula I 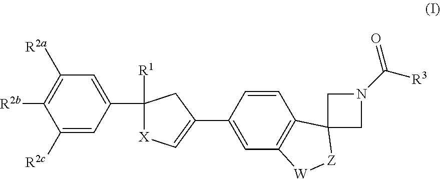
wherein
X CH₂ or S;
W-Z are #-CH₂—O—*, ′#-CH₂—S(O)ₙ—*, or #-C(═O)—O—*, wherein # marks the bond of W to phenyl, and * the bond of Z to azetidin;
n is 0, 1, or 2;
R¹ halomethyl;
R^{2a} is selected from the group consisting of halogen, halomethyl, and halomethoxy;
K^{2b}, R^{2 c}are independently H, or as defined for R^{2a};
R³ is selected from the group consisting of H, C₁-C₆-alkyl, C₁-C₆-haloalkyl, C₂-C₆-alkenyl, C₂-C₆-haloalkenyl, C₂-C₆-alkynyl, C₂-C₆-haloalkynyl which aliphatic groups are unsubstituted or substituted by one or more radicals R³¹; C₃-C₆-cycloalkyl, C₃-C₆-halocycloalkyl which cyclic groups are unsubstituted or substituted by one or more radicals R³²; C(═O)N(R³³)R³⁴, N(R³³)R³⁵, CH═NOR³⁶;
phenyl, heterocycle, or hetaryl which rings are unsubstituted or partially or fully substituted by R^{A};
R³¹ is independently OH, cyano, C₁-C₆-alkoxy, C₁-C₆-haloalkoxy, S(O)ₙ—C₁-C₆-alkyl, S(O)ₙ—C₁-C₆-haloalkyl, C(═O)N(R³³)R^{34,} C₃-C₆-cycloalkyl, or C₃-C₆-halocycloalkyl which cycles are unsubstituted or substituted by one or more _{R}³¹¹; or
phenyl, heterocycle or hetaryl which rings are unsubstituted or partially or fully substituted by R^{A};
R³¹¹ is independently OH, cyano, C₁-C₂-alkyl, or C₁-C₂-haloalkyl;
R³³ is H, or C₁-C₆-alkyl,
R³⁴ is H, C₁-C₆-alkyl, C₁-C₆-haloalkyl, C₂-C₆-alkenyl, C₂-C₆-haloalkenyl, C₂-C₆-alkynyl, C₂-C₆-haloalkynyl, or C₃-C₆-cycloalkyl, C₃-C₆-halocycloalkyl, C₃-C₆-cycloalkylmethyl, or C₃-C₆-halocycloalkylmethyl which rings are unsubstituted or substituted by a cyano;
R³⁵ is selected from the group consisting of H, C₁-C₆-alkyl, C₁-C₆-haloalkyl, C₂-C₄-alkenyl, C₂-C₄-alkynyl, CH₂-CN, C₃-C₆-cycloalkyl, C₃-C₆-halocycloalkyl, C₃-C₆-cycloalkylmethyl, C₃-C₆-halocycloalkylmethyl, phenyl and hetaryl which aromatic rings are unsubstituted or partially or fully substituted by R^{A}; u
R³² is independently C₁-C₆-alkyl, C₁-C₆-haloalkyl, or a group as defined for R³¹;
R³⁶ is independently H, C₁-C₆-alkyl, or C₁-C₆-haloalkyl;
R^{A} is independently selected from halogen, cyano, NO₂, C₁-C₄-alkyl, C₁-C₄-haloalkyl, C₂-C₄-alkenyl, C₂-C₄-haloalkenyl, C₂-C₄-alkynyl, C₂-C₄-haloalkynyl, C₃-C₆-cycloalkyl, C₃-C₆-halocycloalkyl, C₁-C₄-alkoxy, C₁-C₄-haloalkoxy, S(O)ₙ—C₁-C₄- alkyl, S(O)ₙ—C₁-C₄-haloalkyl, C₁-C₄-alkylcarbonyl, C₁-C₄-haloalkylcarbonyl, C(═O)N(R³³)R³⁴; or two R^{A} present on the same carbon atom of a saturated or partially saturated ring may form together ═O or ═S; or two R^{A} present on the same S or SO ring member of a heterocyclic ring may together form a group ═N(C₁-C₆-alkyl), ═NO(C₁-C₆-alkyl), ═NN(H)(C₁-C₆-alkyl) or ═NN(C₁-C₆-alkyl)₂;
and the N-oxides, stereoisomers and agriculturally or veterinarily acceptable salts thereof

17. The compound of formula I according to claim 16, wherein X is S.

18. The compound of formula I according to claim 16, wherein X is CH₂.

19. The compound of formula I according to claim 16, wherein W-Z is #-CH₂—O *

20. The compound of formula I according to claim 16, wherein R¹ is CF₃.

21. The compound of formula I according to claim 16, wherein R^{2a} is selected from the group consisting of F, Cl, Br, CF₃, and OCF₃; and R^{2b} and R^{2c} are independently selected from the group consisting of H, F, Cl, Br, CF₃, and OCF₃.

22. The compound of formula I according to claim 16, wherein
R³ is selected from the group consisting of C₁-C₄-alkyl, and C₁-C₄-haloalkyl, which aliphatic groups are unsubstituted or substituted by one radical R³¹; and C₃-C₅-cycloalkyl, C₃-C₅-halocycloalkyl which cyclic groups are unsubstituted or substituted by one radical R³²;
R³¹ is independently OH, cyano, C₁-C₄-alkoxy, C₁-C₄-haloalkoxy, S(O)ₙ—C₁-C₄-alkyl, S(O)ₙ—C₁-C₄-haloalkyl, C₃-C₅-cycloalkyl, or C₃-C₅-halocycloalkyl which cycles are unsubstituted or substituted by one or more R³¹¹; R³¹¹ is independently OH, cyano, C₁-C₂-alkyl, or C₁-C₂-haloalkyl; n is 0, 1, or 2; and
R³² is independently C₁-C₄-alkyl, C₁-C₄-haloalkyl, or a group as defined for R³¹.

23. The compound of formula I according to claim 16, wherein R³ is selected from the group consisting of C₁-C₃-alkyl, CH₂CF₃, CH₂CH₂CF₃, CH₂OH, CH₂c-C₃H₅, c-C₃H₅, 1-c-CN-c-C₃H₄, 1-CF₃-c-C₃H₄, 1-OH-c-C₃H₄, 2,2,-F₂-c-C₃H₃, CH₂OCH₃, CH₂OC₂H₅, CH₂OCF₃, CH₂OCH₂CF₃, and CH₂S(O)ₙCH₃, CH₂S(O)ₙC₂H₅, wherein n is 0, 1, or 2; 1-pyrazolyl, 3-CH₃-1-pyrazolyl, 2-pyridyl,3-thietan-yl, 3-thietan-yl-S-oxide, and 3-thietan-yl-S-dioxide.

24. A composition comprising at least one compound according to claim 16 and at least one inert liquid and/or solid carrier.

25. An agricultural composition for combating animal pests comprising at least one compound as defined in claim 16 and at least one inert liquid and/or solid acceptable carrier and, if desired, at least one surfactant.

26. The composition according to claim 24, comprising additionally a further active substance.

27. A method for combating or controlling invertebrate pests, which method comprises contacting said pest or its food supply, habitat or breeding grounds with a pesticidally effective amount of at least one compound as defined in claim 16.

28. A method for protecting growing plants from attack or infestation by invertebrate pests, which method comprises contacting a plant, or soil or water in which the plant is growing, with a pesticidally effective amount of at least one compound as defined in claim 16.

29. A seed comprising the compound as defined in claim 16, or the enantiomers, diastereomers or salts thereof, in an amount of from 0.1 g to 10 kg per 100 kg of seed.
